(19) United States
(12) Patent Application Publication
(10) Pub. No.: US 2009/0270418 A1
(43) Pub. Date: Oct. 29, 2009

(54) PYRAZOLE PYRAZINE AMINE COMPOUNDS AS KINASE INHIBITORS, COMPOSITIONS THEREOF AND METHODS OF TREATMENT THEREWITH

(76) Inventors: Marianne Sloss, San Diego, CA (US); Jeffrey McKenna, Horsham (GB); Won Hyung Yoon, San Diego, CA (US); Stephen Norris, San Diego, CA (US); Dale Robinson, Carlsbad, CA (US); Jason Parnes, San Diego, CA (US); Graziella Shevlin, San Diego, CA (US); Paul Erdman, San Diego, CA (US); Robert Hilgraf, San Diego, CA (US); Ronald Albers, San Diego, CA (US); Sayee Hegde, San Diego, CA (US); Andrew Wallace, San Diego, CA (US); Henry Chan, San Diego, CA (US); Loui Madakamutil, San Diego, CA (US); Heather Raymon, San Diego, CA (US)

Correspondence Address: JONES DAY, 222 E. 41ST. STREET, NEW YORK, NY, 10017, US

(21) Appl. No.: 12/350,722

(22) Filed: Jan. 8, 2009

Related U.S. Application Data
(60) Provisional application No. 61/010,866, filed on Jan. 9, 2008.

(51) Int. Cl. A61K 31/497; C07D 403/12; A61P 35/00; C12N 9/99

(52) U.S. Cl. 514/255.05; 544/405; 435/184

(57) ABSTRACT

Provided herein are Pyrazole Pyrazine Amine Compounds having the following structure:

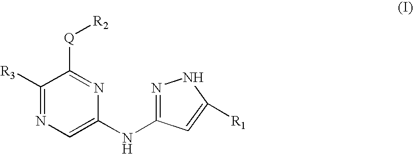

Wherein Q and R¹-R³ are as defined herein, compositions comprising an effective amount of a Pyrazole Pyrazine Amine Compound and methods for treating or preventing inflammatory conditions, immunological conditions, cancer, neurodegenerative diseases, age-related diseases, cardiovascular diseases and metabolic conditions, or conditions treatable or preventable by inhibition of an IKK, or an IKK pathway, comprising administering an effective amount of a Pyrazole Pyrazine Amine Compound to a patient in need thereof.

This application claims the benefit of U.S. provisional application No. 61/010,866, filed Jan. 9, 2008, which is incorporated by reference herein in its entirety

### 1. FIELD

Provided herein are certain Pyrazole Pyrazine Amine compounds, compositions comprising an effective amount of one or more such compounds and methods for treating or preventing cancer, inflammatory conditions, immunological conditions, metabolic conditions and conditions treatable or preventable by inhibition of an IKK, or an IKK pathway, comprising administering an effective amount of a Pyrazole Pyrazine Amine to a patient.

### 2. BACKGROUND

The connection between abnormal protein phosphorylation and the cause or consequence of diseases has been known for over 20 years. Accordingly, protein kinases have become a very important group of drug targets. See Cohen, Nature, 1:309-315 (2002). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001).

The protein kinases are a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many diseases, the most characterized of which include cancer and diabetes. The regulation of signal transduction by cytokines and the association of signal molecules with protooncogenes and tumor suppressor genes have been well documented. Similarly, the connection between diabetes and related conditions, and deregulated levels of protein kinases, has been demonstrated. See e.g., Sridhar et al. Pharmaceutical Research, 17(11):1345-1353 (2000). Viral infections and the conditions related thereto have also been associated with the regulation of protein kinases. Park et al. Cell 101 (7): 777-787 (2000).

Furthermore, protein kinases have become attractive targets for the treatment of cancers. Fabbro et al., Pharmacology & Therapeutics 93:79-98 (2002). It has been proposed that the involvement of protein kinases in the development of human malignancies may occur by: (1) genomic rearrangements (e.g., BCR-ABL in chronic myelogenous leukemia); (2) mutations leading to constitutively active kinase activity, such as acute myelogenous leukemia and gastrointestinal tumors; (3) deregulation of kinase activity by activation of oncogenes or loss of tumor suppressor functions, such as in cancers with oncogenic RAS; (4) deregulation of kinase activity by over-expression, as in the case of EGFR; and (5) ectopic expression of growth factors that can contribute to the development and maintenance of the neoplastic phenotype. Fabbro et al., Pharmacology & Therapeutics 93:79-98 (2002).

NF-κB is a heterodimeric transcription factor regulating the expression of multiple inflammatory genes and has been implicated in many pathophysiologic processes including angiogenesis (Koch et al., Nature 376:517-519 (1995), atherosclerosis (Brand et al., J Clin Inv. 97:1715-1722 (1996), endotoxic shock and sepsis (Bohrer et al., J. Clin. Inv. 100:972-985 (1997), inflammatory bowel disease (Panes et al., Am J Physiol. 269: H1955-H1964 (1995), ischemia/reperfusion injury (Zwacka et al., Nature Medicine 4:698-704 (1998), and allergic lung inflammation (Gosset et al., Int Arch Allergy Immunol. 106:69-77 (1995). Because of the central role of NF-κB in inflammatory disease, inhibition of NF-κB by targeting regulatory proteins in the NF-κB activation pathway represents an attractive strategy for generating anti-inflammatory therapeutics.

The IκB kinases (IKKs) are key regulatory signaling molecules coordinating the activation of NF-κB. Many immune and inflammatory mediators including TNFα, lipopolysaccharide (LPS), IL-1, anti-CD28, CD40L, FasL, viral infection, and oxidative stress have been shown to lead to NF-κB activation. Although the receptor complexes that transduce these diverse stimuli appear very different in their protein components, it is understood that each of these stimulation events leads to activation of the IKKs and NF-κB. The NF-κB heterodimer in its active state is held in the cytoplasm by association with inhibitory IκB proteins (Huxford et al. Cell 95:759-770 (1998); Jacobs et al. Cell 95:749-758 (1998)). Treatment of cells with IL-1 or TNFα leads to activation of intracellular signal transduction pathways that in turn lead to phosphorylation of IκB proteins on specific amino acid residues (serines 32 and 36 in IκB alpha, serines 19 and 23 in IκB beta). Mutation of one or both serine residues renders IκB resistant to cytokine-induced phosphorylation. This signal-induced phosphorylation targets IκB for proteosome-mediated degradation, allowing nuclear translocation of NF-κB (Thanos and Maniatis, Cell 80:529-532 (1995)). The only regulated step in the IκB degradation pathway is the phosphorylation of IκB by IκB kinases (IKK) (Yaron et al. EMBO J. 16:6486-94 (1997)).

The IKK complex appears to be the central integrator of diverse inflammatory signals leading to the phosphorylation of NF-κB. IKKs are activated at dual serine residues by upstream kinases including NF-κB inducing kinase, NIK (Malinin et al., Nature 385:540-544 (1997)), MEKK-1 (Yujiri et al., Science 282:1911-1914 (1998)), MEKK-3 (Yang et al. Nat. Immunol. 2:620-624 (2001)) and TAK1 (Sakurai et al. J Biol. Chem. 274:10641-10648 (1999)).

Although both kinases can phosphorylate NF-κB in vitro, early studies using genetic mutants indicated that IKK-2, but not IKK-1, was essential for activation of NF-κB by pro-inflammatory stimuli such as IL-1β and TNFα. Cell and animal experiments indicate that IKK-2 is a central regulator of the pro-inflammatory role of NF-κB. IKK-2 is activated in response to multiple inflammatory stimuli and signaling pathways, many of which play an important role in respiratory disease including IL-1β, LPS, TNFα, CD3/CD28 (antigen presentation), CD40L, viral infection, and oxidative stress. The ubiquitous expression of NF-κB, along with its response to multiple stimuli means that almost all cell types present are potential targets for anti-NF-κB/IKK-2 therapy. This includes alveolar epithelium, mast cells, fibroblasts, vascular endothelium, and infiltrating leukocytes; neutrophils, macrophages, lympophocytes, eosinophils and basophils. By inhibiting the expression of genes such as cyclooxygenase-2 and 12-lipoxygenase (synthesis of inflammatory mediators), TAP-1 peptide transporter (antigen processing), MHC class I H-2K and class II invariant chains (antigen presentation), E-selectin and vascular cell adhesion molecule (leukocyte recruitment), interleukins-1,2,6, TNFα (cytokines), IL-8, RANTES, eotaxin (chemokines), GM-CSF, and superoxide dismutase and NADPH quinone oxidoreductase (reactive oxygen species), inhibitors of IKK-2 are believed to display broad anti-inflammatory activity.

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on multiple kinases or multiple kinase pathways. Accordingly, there remains a need for new kinase modulators.

Citation or identification of any reference in Section 2 of this application is not to be construed as an admission that the reference is prior art to the present application.

### 3. SUMMARY

Provided herein are compounds having the following formula (I):

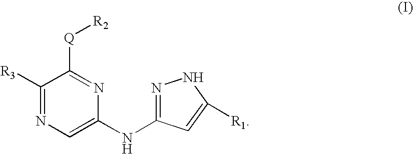

and pharmaceutically acceptable salts, stereoisomers, and tautomers thereof, wherein Q, R¹, R², and R³ are as defined herein.

Compounds of formula (I), or pharmaceutically acceptable salts, stereoisomers, and tautomers thereof (each being referred to herein as a “Pyrazole Pyrazine Amine Compound(s)”), are useful for treating or preventing inflammatory conditions, immunological conditions, cancer, neurodegenerative diseases, age-related diseases, cardiovascular diseases, metabolic conditions, or conditions treatable or preventable by inhibition of an IKK, or an IKK pathway.

Further provided herein are compositions comprising an effective amount of a compound provided herein and compositions comprising such a compound and a pharmaceutically acceptable carrier or vehicle. The compositions are useful for treating or preventing inflammatory conditions, immunological conditions, cancer, neurodegenerative diseases, age-related diseases, cardiovascular diseases, metabolic conditions, or conditions treatable or preventable by inhibition of an IKK, or an IKK pathway.

Further provided herein are methods for treating or preventing inflammatory conditions, immunological conditions, cancer, neurodegenerative diseases, age-related diseases, cardiovascular diseases, metabolic conditions, or conditions treatable or preventable by inhibition of an IKK, or an IKK pathway, comprising administering an effective amount of a compound provided herein to a patient.

Illustrative examples of kinases which compounds provided herein are useful for inhibiting include, but are not limited to, IKK-1 and IKK-2.

Also provided herein is a method of inhibiting an IKK-2 in a cell expressing IKK-2, comprising contacting said cell with an effective amount of a compound provided herein.

The present embodiments can be understood more fully by reference to the detailed description and examples, which are intended to exemplify non-limiting embodiments.

### 4. DETAILED DESCRIPTION

### 4.1 Definitions

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrolyl, pyridyl, pyridazinyl, pyrmidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “aryloxy” group is —O-(aryl), wherein aryl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclylalkyl group as defined herein.

An “acylamino” group is a radical of the formula: —NHC(O)(R^{#}) or —N(alkyl)C(O)(R^{#}), wherein each alkyl and R^{#} are independently as defined above.

An “alkylsulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R), —N(alkyl)C(O)NH₂, —NHC(O)N(R^{#})₂, —NHC(O)NH(R), or —NH(CO)NHR^{#}, wherein each alkyl and R^{#} are independently as defined above.

In one embodiment, when the groups described herein are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl anine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidinyl, piperidinyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “Pyrazole Pyrazine Amine Compound” refers to compounds of formula (I) as well as to further embodiments provided herein. In one embodiment, a “Pyrazole Pyrazine Amine Compound” is a compound set forth in Table 1. The term “Pyrazole Pyrazine Amine Compound” includes pharmaceutically acceptable salts, stereoisomers, and tautomers, of the compounds provided herein.

As used herein and unless otherwise indicated, the term “stereoisomer” or “stereomerically pure” means one stereoisomer of a Pyrazole Pyrazine Amine Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The Pyrazole Pyrazine Amine Compounds can have chiral centers and can occur as racemates, individual enantiomers or diastereomers, and mixtures thereof. All such isomeric forms are included within the embodiments disclosed herein, including mixtures thereof.

Various Pyrazole Pyrazine Amine Compounds contain one or more chiral centers, and can exist as racemic mixtures of enantiomers, mixtures of diastereomers or enantiomerically or optically pure compounds. The use of stereomerically pure forms of such Pyrazole Pyrazine Amine Compounds, as well as the use of mixtures of those forms are encompassed by the embodiments disclosed herein. For example, mixtures comprising equal or unequal amounts of the enantiomers of a particular Pyrazole Pyrazine Amine Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the Pyrazole Pyrazine Amine Compounds can include E and Z isomers, or a mixture thereof, and cis and trans isomers or a mixture thereof. In certain embodiments, the Pyrazole Pyrazine Amine Compounds are isolated as either the E or Z isomer. In other embodiments, the Pyrazole Pyrazine Amine Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

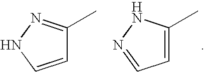

As readily understood by one skilled in the art, a wide variety of functional groups and other structures may exhibit tautomerism and all tautomers of compounds of formula (I) are within the scope of the present invention.

The term “protected” with respect to amine groups, hydroxyl groups, carboxy groups, and sulfhydryl groups refers to forms of these functionalities which are protected from undesirable reaction by means of protecting groups. Protecting groups are known to those skilled in the art and can be added or removed using well-known procedures, such as those set forth in Protective Groups in Organic Synthesis, Greene, T. W.; Wuts, P. G. M., John Wiley & Sons, New York, N.Y., (3rd Edition, 1999). Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methylthiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoyl formate, formate, acetate, trichloroacetate, and trifluoroacetate.

Amine-protecting groups comprise acyl groups such as formyl, acetyl, propionyl, pivaloyl, t-butyl acetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and the like; sulfonyl groups such as benzenesulfonyl, p-toluenesulfonyl. and the like; carbamate forming groups such as benzyl oxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl groups such as benzyl, triphenylmethyl, benzyloxymethyl, and the like; and silyl groups such as trimethylsilyl, and the like. Typical N-protecting groups are formyl, acetyl, benzoyl, pivaloyl, t-butylacetyl, phenylsulfonyl, benzyl, 9-fluorenylmethyloxycarbonyl (Fmoc), t-butyloxycarbonyl (Boc) and benzyloxycarbonyl (Cbz).

Examples of protected sulfhydryl groups include, but are not limited to, thioethers such as S-benzyl thioether, S-t-butylthioether, and S-4-picolyl thioether; substituted S-methyl derivatives such as hemithio, dithio and aminothioacetals; and others.

Representative carboxy protecting groups are C₁ to C₈ alkyl (e.g., methyl, ethyl or tertiary butyl and the like); haloalkyl; alkenyl; cycloalkyl and substituted derivatives thereof such as cyclohexyl, cyclopentyl, and the like; cycloalkylalkyl and substituted derivatives thereof such as cyclohexylmethyl, cyclopentylmethyl, and the like; arylalkyl, for example, phenethyl or benzyl and substituted derivatives thereof such as alkoxybenzyl or nitrobenzyl groups, and the like; arylalkenyl, for example, phenylethenyl and the like; aryl and substituted derivatives thereof, for example, 5-indanyl and the like; dialkylaminoalkyl (e.g. dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl, and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl, and the like; aroyloxyalkyl, such as benzoyloxymethyl, benzoyloxyethyl, and the like; arylalkylcarbonyloxyalkyl, such as benzylcarbonyloxymethyl, 2-benzylcarbonyloxyethyl, and the like; alkoxycarbonylalkyl, such as methoxycarbonylmethyl, cyclohexyloxycarbonylmethyl, 1-methoxycarbonyl-1-ethyl, and the like; alkoxycarbonyloxyalkyl, such as methoxycarbonyloxymethyl, t-butyloxycarbonyloxymethyl, 1-ethoxycarbonyloxy-1-ethyl, 1-cyclohexyloxycarbonyloxy-1-ethyl, and the like; alkoxycarbonylaminoalkyl, such as t-butyloxycarbonylaminomethyl, and the like; alkylaminocarbonylaminoalkyl, such as methylaminocarbonylaminomethyl, and the like; alkanoylaminoalkyl, such as acetylaminomethyl, and the like; heterocycliccarbonyloxyalkyl, such as 4-methylpiperazinylcarbonyloxymethyl, and the like; dialkylaminocarbonylalkyl, such as dimethylaminocarbonylmethyl, diethylaminocarbonylmethyl, and the like; (5-(alkyl)-2-oxo-1,3-dioxolen-4-yl)alkyl, such as (5-t-butyl-2-oxo-1,3-dioxolen-4-yl)methyl, and the like; and (5-phenyl-2-oxo-1,3-dioxolen-4-yl)alkyl, such as (5-phenyl-2-oxo-1,3-dioxolen-4-yl)methyl, and the like.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Pyrazole Pyrazine Amine Compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

The IκB kinase complex is comprised of three subunits each encoded by a separate gene: IKKα (also known as IKK1), IKKβ (also known as IKK2), and IKKγ (also known as NEMO). The α- and β-subunits together are catalytically active whereas the γ-subunit serves a regulatory function. The IKK inhibitory activity of the Pyrazole Pyrazine Amine Compounds can be measured by IKK assays known in the art, for example, the IKK-2 Inhibition Assay as described herein.

“Treating” as used herein, means an alleviation, in whole or in part, of symptoms associated with a disorder or disease, or slowing, or halting of further progression or worsening of those symptoms, or prevention or prophylaxis of the disease or disorder in a subject at risk for developing the disease or disorder.

The term “effective amount” in connection with an Pyrazole Pyrazine Amine Compound can mean an amount capable of alleviating, in whole or in part, symptoms associated with a disorder or disease, or slowing or halting further progression or worsening of those symptoms, or preventing or providing prophylaxis for the disease or disorder in a subject having or at risk for developing a disease disclosed herein, such as cancer, inflammatory conditions, immunological conditions, metabolic conditions or conditions treatable or preventable by inhibition of an IKK, or an IKK pathway.

The term “cancer” refers to any of various malignant neoplasms characterized by the proliferation of cells that can invade surrounding tissue and metastasize to new body sites. Both benign and malignant tumors are classified according to the type of tissue in which they are found. For example, fibromas are neoplasms of fibrous connective tissue, and melanomas are abnormal growths of pigment (melanin) cells. Malignant tumors originating from epithelial tissue, e.g., in skin, bronchi, and stomach, are termed carcinomas. Malignancies of epithelial glandular tissue such as are found in the breast, prostate, and colon, are known as adenocarcinomas. Malignant growths of connective tissue, e.g., muscle, cartilage, lymph tissue, and bone, are called sarcomas. Lymphomas and leukemias are malignancies arising among the white blood cells.

In the context of neoplasm, cancer, tumor growth or tumor cell growth, inhibition may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition is referred to herein as prevention or chemoprevention In this context, the term “prevention” includes either preventing the onset of clinically evident neoplasia altogether or preventing the onset of a preclinically evident stage of neoplasia in individuals at risk. Also intended to be encompassed by this definition is the prevention of transformation into malignant cells or to arrest or reverse the progression of premalignant cells to malignant cells. This includes prophylactic treatment of those at risk of developing the neoplasia.

The term “subject” or “patient” includes an animal, including, but not limited to, an animal such as a cow, monkey, horse, sheep, pig, chicken, turkey, quail, cat, dog, mouse, rat, rabbit or guinea pig, in one embodiment a mammal, in another embodiment a human. In a particular embodiment, the patient is in need of the treatment or prevention of a disease disclosed herein, such as cancer, inflammatory conditions, immunological conditions, metabolic conditions and conditions treatable or preventable by inhibition of an IKK, or an IKK pathway or a symptom thereof.

### 4.2 Substituted Pyridine Compounds

Provided herein are Pyrazole Pyrazine Amine Compounds having the following formula (I):

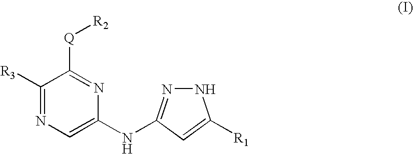

and pharmaceutically acceptable salts, stereoisomers or tautomers thereof, wherein:

Q is a direct bond or NH;

R¹ is H or a substituted or unsubstituted (C₁₋₄)alkyl;

R² is cycloalkyl; aryl; or heterocyclyl, wherein the cycloalkyl, aryl, or heterocyclyl is optionally substituted with one or more substituted or unsubstituted C₁₋₆ alkyl; cyano; halogen; (C₁₋₆)alkoxy; aryloxy; acylamino; aminocarbonyl; urea; (C₁₋₆)alkylsulfonylamino; NR⁴₂, C(O)OR⁵; C(O)R⁶; OC(O)R⁷; NRC(O)OR⁸; or a substituted or unsubstituted heterocyclyl;

R³ is H, CN, C(O)NR⁹R¹⁰, C(O)OR⁹, or C(O)R¹¹;

R⁴ is at each occurrence independently H, substituted or unsubstituted C₁₋₆ alkyl, or substituted or unsubstituted C₁₋₆ cycloalkyl;

R⁵, R⁶, R⁷ and R⁸ at each occurrence are independently substituted or unsubstituted C₁₋₆ alkyl, or substituted or unsubstituted C₁₋₆ cycloalkyl;

R⁹ and R¹⁰ are each independently H, substituted or unsubstituted C₁₋₆ alkyl, substituted or unsubstituted C₁₋₆ cycloalkyl, substituted or unsubstituted heterocyclyl, or substituted or unsubstituted heterocyclylalkyl; or R⁹ and R¹⁰, together with the nitrogen atom to which they are attached form a substituted or unsubstituted heterocyclyl;

R¹¹ is substituted or unsubstituted C₁₋₆ alkyl, or substituted or unsubstituted C₁₋₆ cycloalkyl; and

R is H or substituted or unsubstituted C₁₋₄ alkyl;

provided that the compound is not N-(5-methyl-1H-pyrazol-3-yl)-6-(pyridin-2-yl)pyrazin-2-amine.

In some embodiments of compounds of formula (I), Q is NH. In others, R¹ is methyl.

In some embodiments of compounds of formula (I), R² is a cycloalkyl, aryl, or heterocyclyl selected from cyclohexyl, phenyl, pyridinyl, quinolyl, isoquinolyl, tetrahydroquinolyl, tetrahydroisoquinolyl, dihydroquinolinonyl, dihydroisoquinolinonyl, benzoxazinone, dihydroindenyl, dihydroindenonyl, dihydrobenzazepinone, tetrahydrobenzazepine, benzothiazolyl, benzodioxolyl, indazolyl, benzimidazolyl, indolinyl, isoindolinyl, and indolinonyl.

In some embodiments, R² is phenyl, dihydroindenyl, dihydroindenonyl, substituted with one or more substituted or unsubstituted C₁₋₆ alkyl; cyano; halogen; (C₁₋₆)alkoxy; acylamino; aminocarbonyl; urea; (C₁₋₆)alkylsulfonylamino; NR⁴₂; C(O)OR⁵; C(O)R⁶; OC(O)R⁷; or NRC(O)OR⁸. In others, R² is phenyl, dihydroindenyl, dihydroindenonyl, substituted with one or more C₁₋₆ alkyl; cyano; halogen; O(C₁₋₄alkyl); NHC(O)(C₁₋₄ alkyl); N(C₁₋₄ alkyl)C(O)(C₁₋₄ alkyl); NHC(O)(C₁₋₆ cycloalkyl); N(C₁₋₄ alkyl)C(O)(C₁₋₆ cycloalkyl); NHC(O)(heterocyclyl); N(C₁₋₄ alkyl)C(O)(heterocyclyl); C(O)NH(C₁₋₄ alkyl); C(O)N(C₁₋₄ alkyl)₂; NHC(O)NH(C₁₋₄ alkyl); N(C₁₋₄ alkyl)C(O)NH(C₁₋₄ alkyl); NHC(O)N(C₁₋₄ alkyl)₂; NHC(O)NH(C₁₋₄ cycloalkyl); N(C₁₋₄ alkyl)C(O)NH(C₁₋₄ cycloalkyl); NHC(O)NH(aryl); NHSO₂(C₁₋₄ alkyl); N(C₁₋₄ alkyl)SO₂(C₁₋₄ alkyl); NHSO₂(aryl); NH(C₁₋₄ alkyl); N(C₁₋₄ alkyl)₂; NH(C₁₋₄ cycloalkyl); N(C₁₋₄ alkyl)(C₁₋₄ cycloalkyl); C(O)O(C₁₋₄ alkyl); OC(O)(C₁₋₄ alkyl); NHC(O)O(C₁₋₄ alkyl); N(C₁₋₄ alkyl)C(O)O(C₁₋₄ alkyl); NHC(O)O(C₁₋₆ cycloalkyl); or N(C₁₋₄ alkyl)C(O)O(C₁₋₆ cycloalkyl); wherein each alkyl, cycloalkyl or heterocyclyl is substituted or unsubstituted. For example, R² is phenyl, dihydroindenyl, dihydroindenonyl, substituted with one or more Cl, F, CN, CF₃, methyl, ethyl, n-propyl, isopropyl, n-butyl, isobutyl, sec-butyl, t-butyl, O-methyl, O-ethyl, (C₁₋₄ alkyl)-OH, (CH₂)ₙC(O)OR, (CH₂)ₙC(O)NR₂, (CH₂)ₙO(C₁₋₄ alkyl), NHC(O)CH₃, NHC(O)CH₂CH₃; NHC(O)CH(CH₃)₂, NHC(O)cyclopropyl, NHC(O)cyclobutyl, NHC(O)cyclopentyl, N(CH₃)C(O)CH₃, NHC(O)(CH₂)ₙOR, NHC(O)(CH₂)ₙNR₂, NHC(O)(pyrrolidinyl), NHC(O)(morpholinyl), C(O)NHCH₃, C(O)N(CH₃)₂, NHC(O)NH(CH₃), NHC(O)NH(CH₂CH₃), NHC(O)NH(CH(CH₃)₂), NHC(O)NH(cyclopentyl), NHC(O)NH(cyclohexyl), NHC(O)NH(phenyl), NHC(O)N(CH₃)₂, NHSO₂(CH₃), NHSO₂(phenyl), NH(CH₃), N(CH₃)₂, NH(cyclopropyl), NH(cyclobutyl), NH(cyclopentyl), C(O)O(CH₃), OC(O)(CH₃), NHC(O)O(CH₃), NHC(O)O(CH₂CH₃), NHC(O)O(CH₂(CH₃)₂), NHC(O)O(cyclopropyl), NHC(O)O(cyclobutyl), NHC(O)O(cyclopentyl), N(CH₃)C(O)O(CH₃), wherein R is H or substituted or unsubstituted C₁₋₆ alkyl, and n is 1-3.

In some embodiments of compounds of formula (I), R² is phenyl, dihydroindenyl, dihydroindenonyl, substituted with at least one methyl, Cl or F.

In some such embodiments, R² is phenyl, dihydroindenyl, dihydroindenonyl, substituted with NHC(O)CH₃, NHC(O)CH₂CH₃; NHC(O)CH(CH₃)₂, NHC(O)cyclopropyl, NHC(O)cyclobutyl, NHC(O)cyclopentyl, N(CH₃)C(O)CH₃, NHC(O)(CH₂)ₙOR, NHC(O)(CH₂)ₙNR₂, NHC(O)(pyrrolidinyl), NHC(O)(morpholinyl), NHC(O)NH(CH₃), NHC(O)NH(CH₂CH₃), NHC(O)NH(CH(CH₃)₂), NHC(O)NH(cyclopentyl), NHC(O)NH(cyclohexyl), NHC(O)NH(phenyl), NHC(O)N(CH₃)₂, NHC(O)O(CH₃), NHC(O)O(CH₂CH₃), NHC(O)O(CH₂(CH₃)₂), NHC(O)O(cyclopropyl), NHC(O)O(cyclobutyl), NHC(O)O(cyclopentyl), N(CH₃)C(O)O(CH₃). In some such embodiments, Q is NH. In others, R³ is H.

In some embodiments of compounds of formula (I), R² is phenyl, dihydroindenyl, dihydroisoindenonyl, substituted with a substituted or unsubstituted heterocyclyl. In some such embodiments, the heterocyclyl is pyrrolidinyl, pyrrolidinonyl, oxazolidinonyl, or piperidonyl. For example, the heterocyclyl is

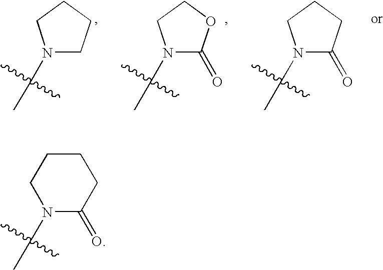

In some such embodiments, R² is phenyl, dihydroindenyl, dihydroindenonyl, substituted with at least one methyl, Cl or F.

In some such embodiments, Q is NH. In others, R³ is H.

In some embodiments of compounds of formula (I), R² is a heterocyclyl, optionally substituted with substituted or unsubstituted C₁₋₆ alkyl; (C₁₋₄)alkoxy; aminocarbonyl; C(O)OR⁵; or C(O)R⁶. In some embodiments, R² is a bicyclic heterocyclyl. In others, R² is pyridinyl, quinolyl, isoquinolyl, tetrahydroquinolyl, tetrahydroisoquinolyl, dihydroquinolinonyl, dihydroisoquinolinonyl, benzoxazinone, dihydroindenyl, dihydroindenonyl, dihydrobenzazepinone, tetrahydrobenzazepine, benzothiazolyl, benzodioxolyl, indazolyl, benzimidazolyl, indolinyl, isoindolinyl, and indolinonyl. In some such embodiments, the heterocyclyl is substituted with methyl, ethyl, n-propyl, isopropyl, O(C₁₋₃ alkyl), (CH₂)ₘOR, (CH₂)ₘOC(O)(C₁₋₆ cycloalkyl), C(O)NH(C₁₋₄ alkyl), C(O)N(C₁₋₄ alkyl)₂, C(O)O(C₁₋₆ alkyl), C(O)(C₁₋₄ alkyl), or C(O)(C₁₋₆ cycloalkyl), wherein R is H or substituted or unsubstituted C₁₋₆ alkyl, and m is 1-3. For example, the heterocyclyl is substituted with methyl, ethyl, n-propyl, isopropyl, O— methyl, O-ethyl, (CH₂)OH, (CH₂)OC(O)CH₃, C(O)NH(CH₃), C(O)N(CH₃)₂, C(O)OCH₃, C(O)CH₃, C(O)(cyclopropyl), C(O)(cyclobutyl), or C(O)(cyclopentyl).

In some embodiments of compounds of formula (I), R² is

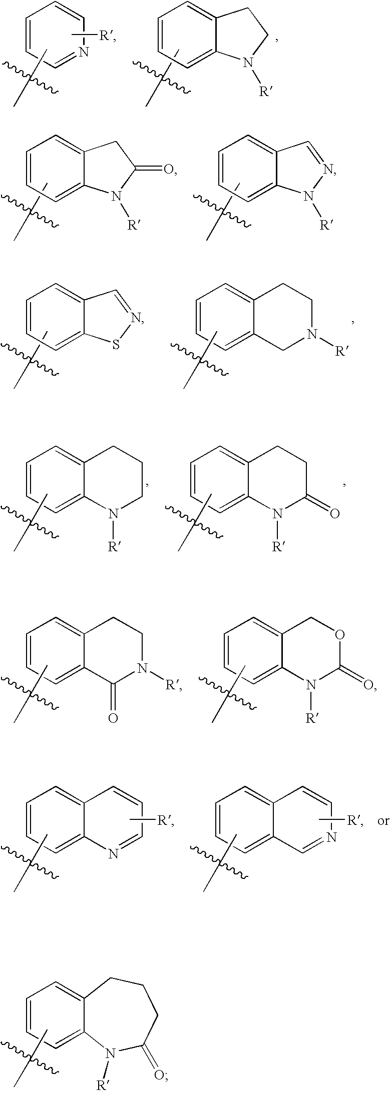

wherein R′ is H, methyl, ethyl, n-propyl, isopropyl, O(C₁₋₃ alkyl), (CH₂)ₘOR, (CH₂)ₘOC(O)C₁₋₄ cycloalkyl), C(O)NH(C₁₋₄ alkyl), C(O)N(C₁₋₆ alkyl)₂, C(O)O(C₁₋₆ alkyl), C(O)(C₁₋₄ alkyl), or C(O)(C₁₋₆ cycloalkyl), wherein R is H or substituted or unsubstituted C₁₋₆ alkyl, and m is 1-3.

In some such embodiments, Q is NH.

In other such embodiments, R³ is H.

In some embodiments of compounds of formula (I), R³ is CN, C(O)NR⁹R¹⁰, or C(O)R¹¹. In some such embodiments, R⁹ and R¹⁰ are each independently H, substituted or unsubstituted C₁₋₆ alkyl, substituted or unsubstituted C₁₋₆ cycloalkyl, substituted or unsubstituted heterocyclyl, or substituted or unsubstituted heterocyclylalkyl. For example, R⁹ and R¹⁰ are each independently H, methyl, ethyl, n-propyl, isopropyl, (CH₂)ₚNR₂, (CH₂)ₚNRC(O)R, (CH₂)ₚCONR₂, (CH₂)ₚOR; substituted or unsubstituted cyclopropyl, cyclobutyl, cyclopentyl, or cyclohexyl; substituted or unsubstituted azetidinyl, pyrrolidinyl, pyrrolidinonyl, piperidinyl or piperazinyl; or substituted or unsubstituted (CH₂)ₚ(azetidinyl), (CH₂)ₚ(pyrrolidinyl), (CH₂)ₚ(pyrrolidinonyl), (CH₂)ₚ(piperidinyl), or (CH₂)ₚ(piperazinyl); wherein each R is independently H or substituted or unsubstituted C₁₋₄ alkyl, and p is 1-3.

In some such embodiments, R⁹ and R¹⁰ together with the nitrogen atom to which they are attached form a substituted or unsubstituted heterocyclyl, for example, pyrrolidinyl, pyrrolidinonyl, piperidinyl, piperidonyl, piperazinyl, or piperazinonyl.

In other such embodiments, R¹¹ is substituted or unsubstituted C₁₋₄ alkyl.

In some embodiments of compounds of formula (I), R³ is

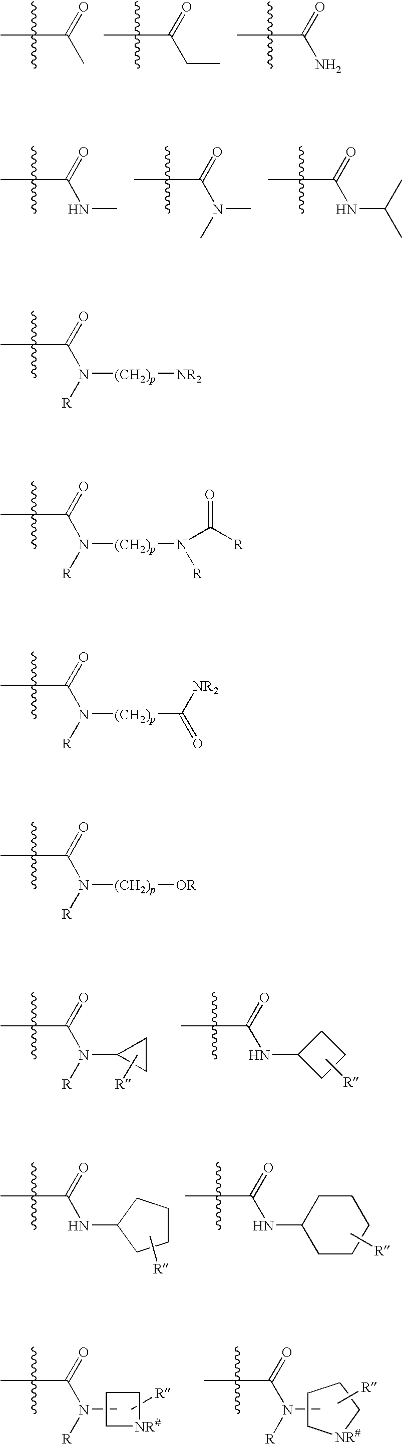

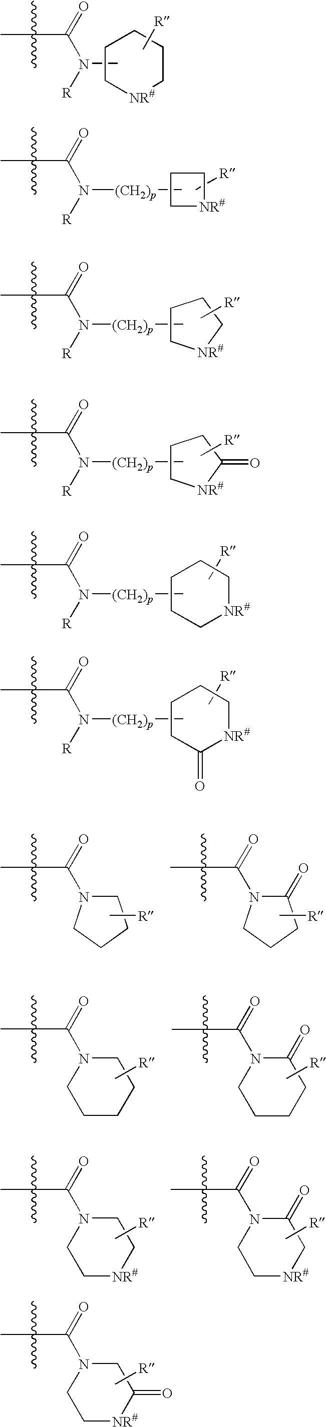

wherein each R is independently H or substituted or unsubstituted C₁₋₆ alkyl, R″ is H, C(O)C₁₋₄ alkyl), wherein the alkyl is optionally substituted or unsubstituted, R^{#} is H, NR₂, OR, (CH₂)ₚNR₂, (CH₂)ₚOR, or NR(CO)(C₁₋₄ alkyl), and p is 1-3.

In some such embodiments, Q is NH.

Any and all combinations resulting from each of the above embodiments are also contemplated by the current disclosure.

Representative Pyrazole Pyrazine Amine Compounds are set forth in Table 1, below.

TABLE 1

| Cmpd. | | | Obs. |
|---|---|---|---|
| No. | Cmpd. Structure | Cmpd. Name | MS |
| 1 | 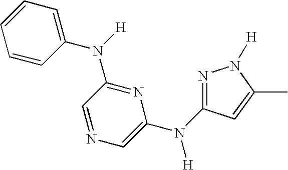 | N²-(5-Methyl-1H-pyrazol-3-yl)-N⁶- phenylpyrazine-2,6-diamine | 267.3 |
| 2 | 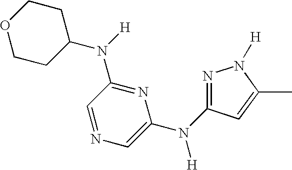 | N²-(5-methyl-1H-pyrazol-3-yl)-N⁶- (tetrahydro-2H-pyran-4-yl)pyrazine- 2,6-diamine | 275.3 |
| 3 | 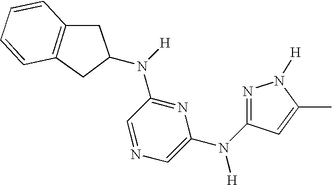 | N²-(2,3-dihydro-1H-inden-2-yl)-N⁶- (5-methyl-1H-pyrazol-3-yl)pyrazine- 2,6-diamine | 307.4 |
| 4 | 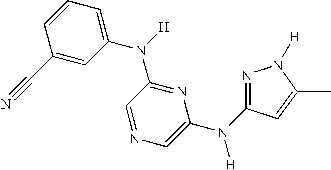 | 3-(6-(5-methyl-1H-pyrazol-3- ylamino)pyrazin-2- ylamino)benzonitrile | 292.1 |
| 5 | 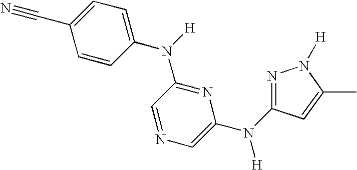 | 4-(6-(5-methyl-1H-pyrazol-3- ylamino)pyrazin-2- ylamino)benzonitrile | 292.1 |
| 6 | 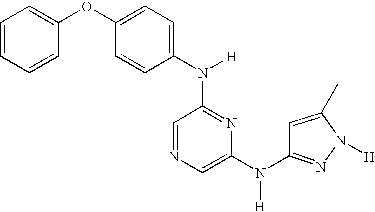 | 6-(5-methyl-1H-pyrazol-3-yl)-N-(4- phenoxyphenyl)pyrazin-2-amine | 359.1 |
| 7 | 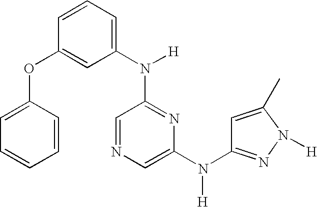 | 6-(5-methyl-1H-pyrazol-3-yl)-N-(3- phenoxyphenyl)pyrazin-2-amine | 359.1 |
| 8 | 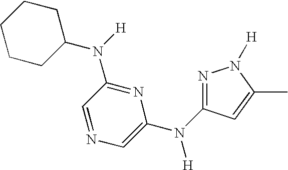 | N²-cyclohexyl-N⁶-(5-methyl-1H- pyrazol-3-yl)pyrazine-2,6-diamine | 273.4 |
| 9 | 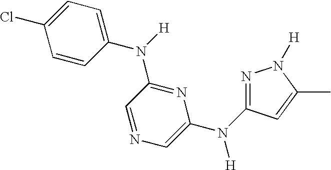 | N²-(4-chlorophenyl)-N⁶-(5-methyl- 1H-pyrazol-3-yl)pyrazine-2,6- diamine | 301.2 |
| 10 | 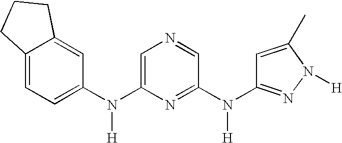 | N²-(2,3-dihydro-1H-inden-5-yl)-N⁶- (5-methyl-1H-pyrazol-3-yl)pyrazine- 2,6-diamine | 307.2 |
| 11 | 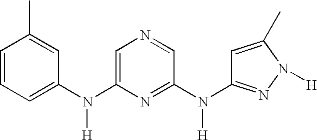 | N²-(5-methyl-1H-pyrazol-3-yl)-N⁶- m-tolylpyrazine-2,6-diamine | 281.2 |
| 12 | 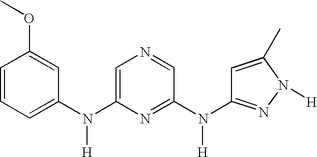 | N²-(3-methoxyphenyl)-N⁶-(5- methyl-1H-pyrazol-3-yl)pyrazine- 2,6-diamine | 297.1 |
| 13 | 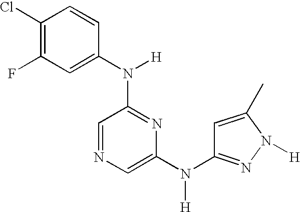 | N²-(4-chloro-3-fluorophenyl)-N⁶-(5- methyl-1H-pyrazol-3-yl)pyrazine- 2,6-diamine | 319.1 |
| 14 | 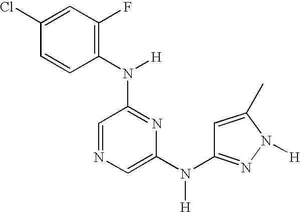 | N²-(4-chloro-2-fluorophenyl)-N⁶-(5- methyl-1H-pyrazol-3-yl)pyrazine- 2,6-diamine | 319.1 |
| 15 | 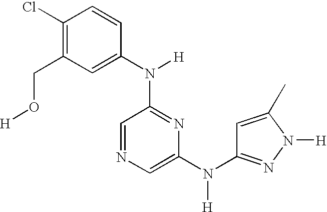 | (2-chloro-5-(6-(5-methyl-1H- pyrazol-3-ylamino)pyrazin-2- ylamino)phenyl)methanol | 331.1 |
| 16 | 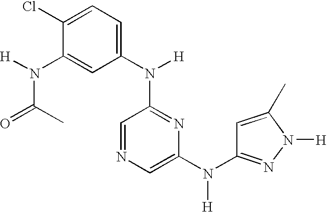 | N-(2-chloro-5-(6-(5-methyl-1H- pyrazol-3-ylamino)pyrazin-2- ylamino)phenyl)acetamide | 358.1 |
| 17 | 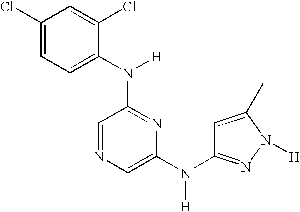 | N²-(2,4-dichlorophenyl)-N⁶-(5- methyl-1H-pyrazol-3-yl)pyrazine- 2,6-diamine | 335.1 |
| 18 | 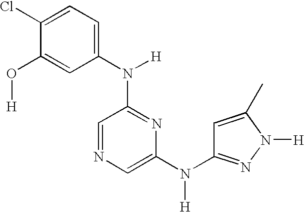 | 2-chloro-5-(6-(5-methy1-1H-pyrazol- 3-ylamino)pyrazin-2-ylamino)phenol | 317.1 |
| 19 | 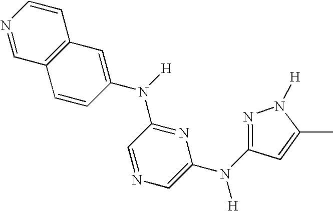 | N²-(isoquinolin-6-yl)-N⁶-(5-methyl- 1H-pyrazol-3-yl)pyrazine-2,6- diamine | 318.3 |
| 20 | 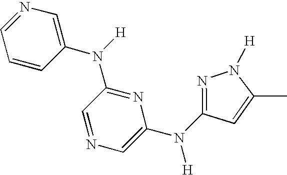 | N²-(5-methyl-1H-pyrazol-3-yl)-N⁶- (pyridin-3-yl)pyrazine-2,6-diamine | 268.1 |
| 21 | 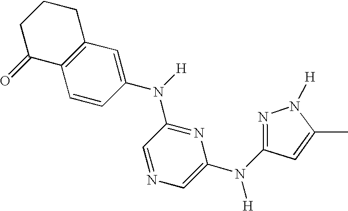 | 6-(6-(5-methyl-1H-pyrazol-3- ylamino)pyrazin-2-ylamino)-3,4- dihydronaphthalen-1(2H)-one | 335.4 |
| 22 | 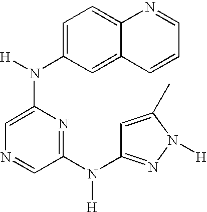 | N²-(5-methyl-1H-pyrazol-3-yl)-N⁶- (quinolin-6-yl)pyrazine-2,6-diamine | 318.1 |
| 23 | 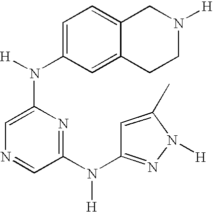 | N²-(5-methyl-1H-pyrazol-3-yl)-N⁶- (1,2,3,4-tetrahydroisoquinolin-6- yl)pyrazine-2,6-diamine | 322.2 |
| 24 | 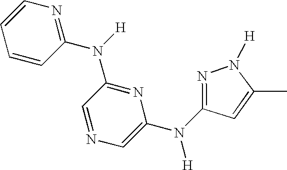 | N²-(5-methyl-1H-pyrazol-3-yl)-N⁶- (pyridin-2-yl)pyrazine-2,6-diamine | 268.1 |
| 25 | 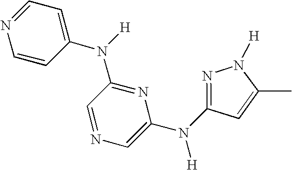 | N²-(5-methyl-1H-pyrazol-3-yl)-N⁶- (pyridin-4-yl)pyrazine-2,6-diamine | 268.1 |
| 26 | 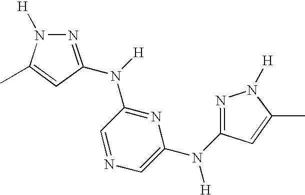 | N²,N⁶-bis(5-methyl-1H-pyrazol-3- yl)pyrazine-2,6-diamine | 271.2 |
| 27 | 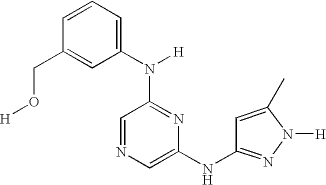 | (3-(6-(5-methyl-1H-pyrazol-3- ylamino)pyrazin-2- ylamino)phenyl)methanol | 297.1 |
| 28 | 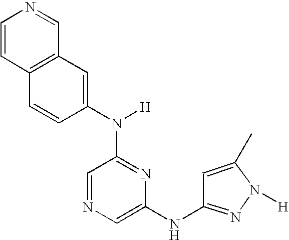 | N²-(isoquinolin-7-yl)-N⁶-(5-methyl- 1H-pyrazo1-3-yl)pyrazine-2,6- diamine | 318.1 |
| 29 | 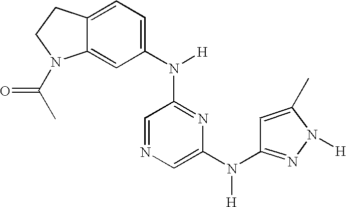 | 1-(6-(6-(5-methyl-1H-pyrazol-3- ylamino)pyrazin-2˜ylamino)indolin- 1-yl)ethanone | 350.1 |
| 30 | 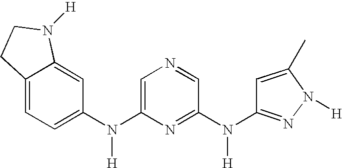 | N²-(indolin-6-yl)-N⁶-(5-methyl-1H- pyrazol-3-yl)pyrazine-2,6-diamine | 308.1 |
| 31 | 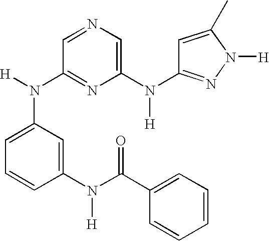 | N-(3-(6-(5-methyl-1H-pyrazol-3- ylamino)pyrazin-2- ylamino)phenyl)benzamide | 387.5 |
| 32 | 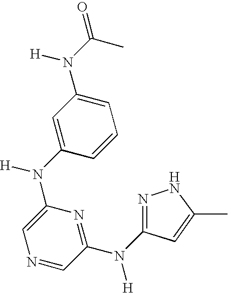 | N-(3-(6-(5-methyl-1H-pyrazol-3- ylamino)pyrazin-2- ylamino)phenyl)acetamide | 324.3 |
| 33 | 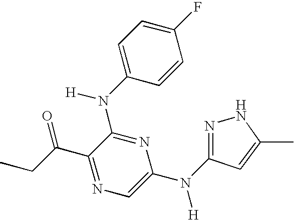 | 1-(3-(4-fluorophenylamino)-5-(5- methyl-1H-pyrazol-3- ylamino)pyrazin-2-yl)propan-1-one | 341.3 |
| 34 | 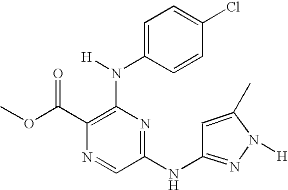 | methyl 3-(4-chlorophenylamino)-5- (5-methyl-1H-pyrazol-3- ylamino)pyrazine-2-carboxylate | 359.1 |
| 35 | 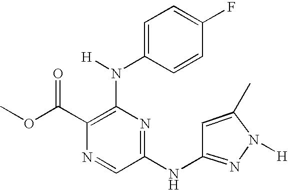 | methyl 3-(4-fluorophenylamino)-5- (5-methyl-1H-pyrazol-3- ylamino)pyrazine-2-carboxylate | 343 |
| 36 | 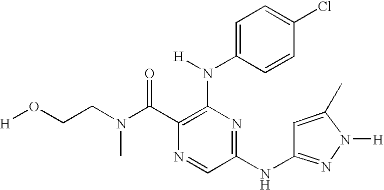 | 3-(4-chlorophenylamino)-N-(2- hydroxyethyl)-N-methyl-5-(5- methyl-1H-pyrazol-3- ylamino)pyrazine-2-carboxamide | 402.2 |
| 37 | 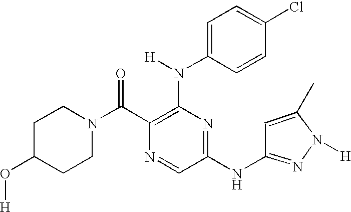 | (3-(4-chlorophenylamino)-5-(5- methyl-1H-pyrazol-3- ylamino)pyrazin-2-yl)(4- hydroxypiperidin-1-yl)methanone | 428.5 |
| 38 | 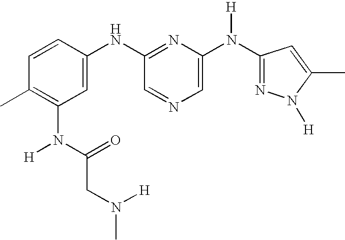 | N-(2-methyl-5-(6-(5-methyl-1H- pyrazol-3-ylamino)pyrazin-2- ylamino)phenyl)-2- (methylamino)acetamide | 367.2 |
| 39 | 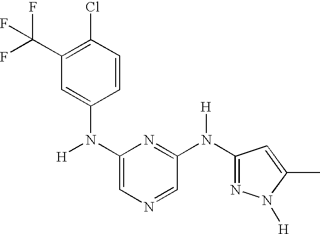 | N²-(4-chloro-3- (trifluoromethyl)phenyl)-N⁶-(5- methyl-1H-pyrazol-3-yl)pyrazine- 2,6-diamine | 369.1 |
| 40 | 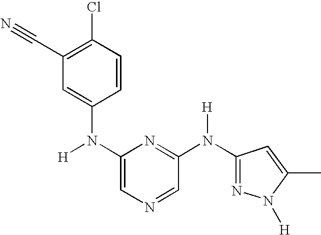 | 2-chloro-5-(6-(5-methyl-1H-pyrazol- 3-ylamino)pyrazin-2- ylamino)benzonitrile | 326.1 |
| 41 | 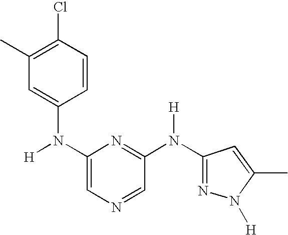 | N²-(4-chloro-3-methylphenyl)-N⁶- (5-methyl-1H-pyrazol-3-yl)pyrazine- 2,6-diamine | 315.2 |
| 42 | 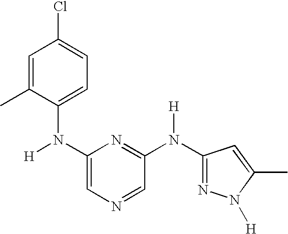 | N²-(4-chloro-2-methylphenyl)-N⁶- (5-methyl-1H-pyrazol-3-yl)pyrazine- 2,6-diamine | 315.2 |
| 43 | 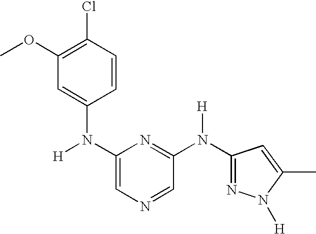 | N²-(4-chloro-3-methoxyphenyl)-N⁶- (5-methyl-1H-pyrazol-3-yl)pyrazine- 2,6-diamine | 331.1 |
| 44 | 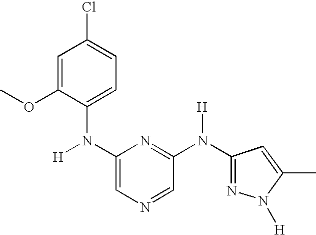 | N²-(4-chloro-2-methoxyphenyl)-N⁶- (5-methyl-1H-pyrazol-3-yl)pyrazine- 2,6-diamine | 331.1 |
| 45 | 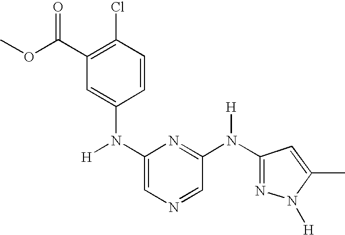 | methyl 2-chloro-5-(6-(5-methyl-1H- pyrazol-3-ylamino)pyrazin-2- ylamino)benzoate | 359.1 |
| 46 | 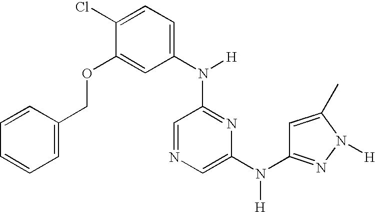 | N²-(3-(benzyloxy)-4-chlorophenyl)- N⁶-(5-methyl-1H-pyrazol-3- yl)pyrazine-2,6-diamine | 407.1 |
| 47 | 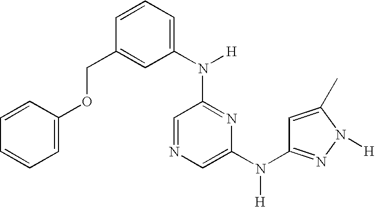 | N²-(5-methyl-1H-pyrazol-3-yl)-N⁶- (3-(phenoxymethyl)phenyl)pyrazine- 2,6-diamine | 373.1 |
| 48 | 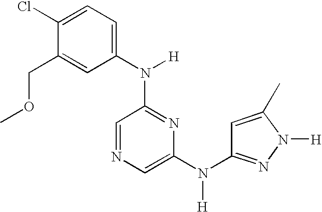 | N²-(4-chloro-3- (methoxymethyl)phenyl)-N⁶-(5- methyl-1H-pyrazo1-3-yl)pyrazine- 2,6-diamine | 345.2 |
| 49 | 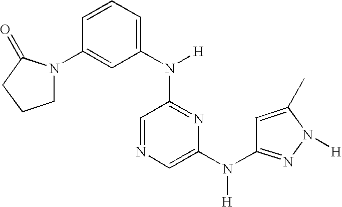 | 1-(3-(6-(5-methyl-1H-pyrazol-3- ylamino)pyrazin-2- ylamino)phenyl)pyrrolidin-2-one | 350.1 |
| 50 | 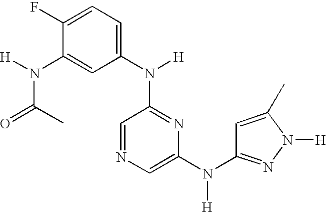 | N-(2-fluoro-5-(6-(5-methyl-1H- (pyrazol-3-ylamino)pyrazin-2- ylamino)phenyl)acetamide | 342.2 |
| 51 | 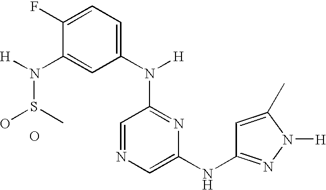 | N-(2-fluoro-5-(6-(5-methyl-1H- pyrazol-3-ylamino)pyrazin-2- ylamino)phenyl)methanesulfonamide | 378.2 |
| 52 | 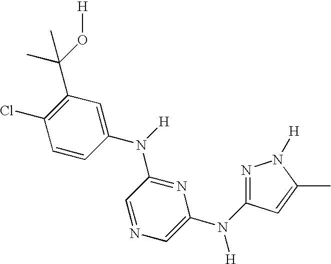 | 2-(2-chloro-5-(6-(5-methyl-1H- pyrazo-3-ylamino)pyrazin-2- ylamino)phenyl)propan-2-ol | 359.4 |
| 53 | 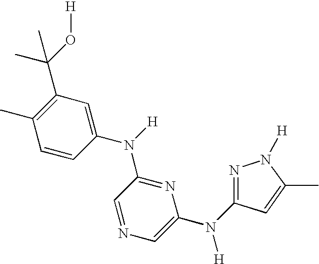 | 2-(2-methyl-5-(6-(5-methyl-1H- pyrazol-3-ylamino)pyrazin-2- ylamino)phenyl)propan-2-ol | 339.5 |
| 54 | 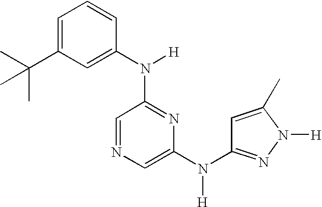 | N²-(3-tert-butylphenyl)-N⁶-(5- methyl-1H-pyrazol-3-yl)pyrazine- 2,6-diamine | 323.3 |
| 55 | 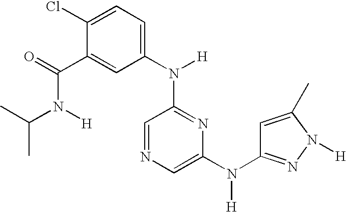 | 2-chloro-N-isopropyl-5-(6-(5- methyl-1H-pyrazol-3- ylamino)pyrazin-2- ylamino)benzamide | 386.1 |
| 56 | 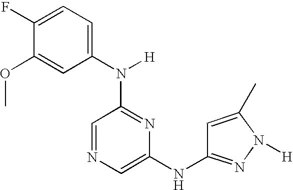 | N²-(4-fluoro-3-methoxyphenyl)-N⁶- (5-methyl-1H-pyrazo1-3-yl)pyrazine- 2,6-diamine | 315.1 |
| 57 | 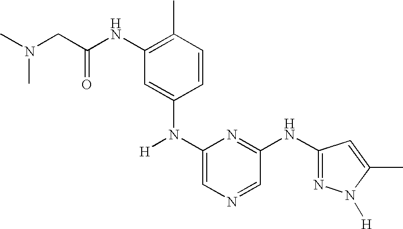 | 2-(dimethylamino)-N-(2-methyl-5- (6-(5-methyl-1H-pyrazol-3- ylamino)pyrazin-2- ylamino)phenyl)acetamide | 381.3 |
| 58 | 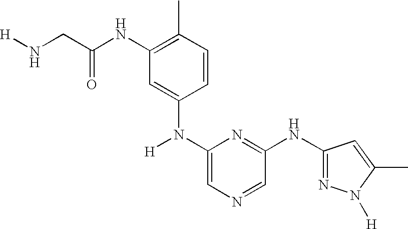 | 2-amino-N-(2-methyl-5-(6-(5- methyl-1H-pyrazol-3- ylamino)pyrazin-2- ylamino)phenyl)acetamide | 353.2 |
| 59 | 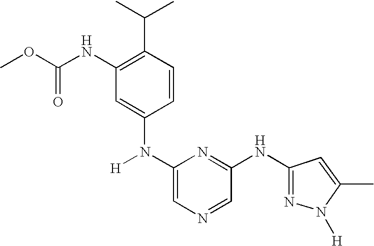 | methyl 2-isopropyl-5-(6-(5-methyl- 1H-pyrazol-3-ylamino)pyrazin-2- ylamino)phenylcarbamate | 382.2 |
| 60 | 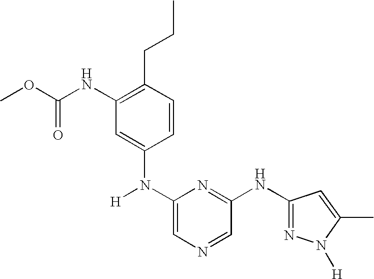 | methyl 5-(6-(5-methyl-1H-pyrazol-3- ylamino)pyrazin-2-ylamino)-2- propylphenylcarbamate | 382.2 |
| 61 | 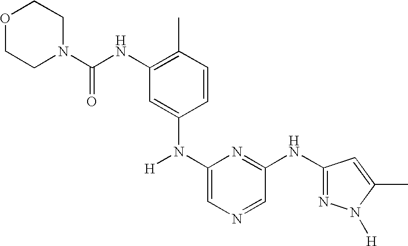 | N-(2-methyl-5-(6-(5-methyl-1H- pyrazol-3-ylamino)pyrazin-2- ylamino)phenyl)morpholine-4- carboxamide | 409.4 |
| 62 | 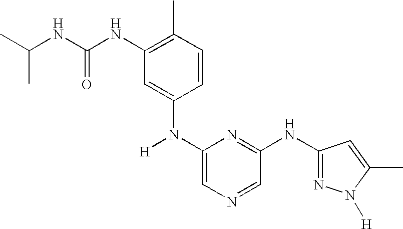 | 1-isopropyl-3-(2-methyl-5-(6-(5- methyl-1H-pyrazol-3- ylamino)pyrazin-2- ylamino)phenyl)urea | 381.3 |
| 63 | 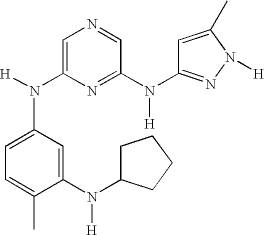 | N²-(3-(cyclopentylamino)-4- methylphenyl)-N⁶-(5-methyl-1H- pyrazol-3-yl)pyrazine-2,6-diamine | 364.1 |
| 64 | 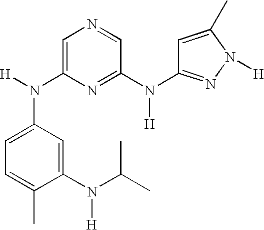 | N²-(3-(isopropylamino)-4- methylphenyl)-N⁶-(5-methyl-1H- pyrazol-3-yl)pyrazine-2,6-diamine | 338.5 |
| 65 | 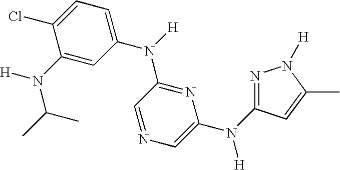 | N²-(4-chloro-3- (isopropylamino)phenyl)-N⁶-(5- methyl-1H-pyrazol-3-yl)pyrazine- 2,6-diamine | 358.4 |
| 66 | 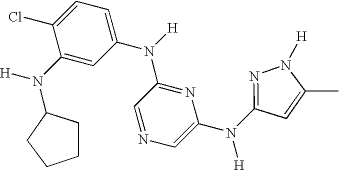 | N²-(4-chloro-3- (cyclopentylamino)phenyl)-N⁶-(5- methyl-1H-pyrazol-3-yl)pyrazine- 2,6-diamine | 384.1 |
| 67 | 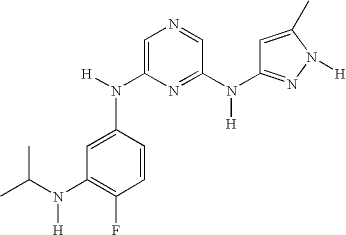 | N²-(4-fluoro-3- (isopropylamino)phenyl)-N⁶-(5- methyl-1H-pyrazol-3-yl)pyrazine- 2,6-diamine | 342.2 |
| 68 | 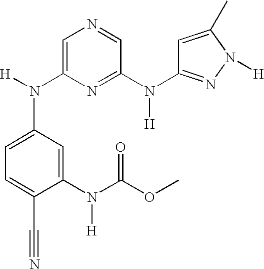 | methyl 2-cyano-5-(6-(5-methyl-1H- pyrazol-3-ylamino)pyrazin-2- ylamino)phenylcarbamate | 365.3 |
| 69 | 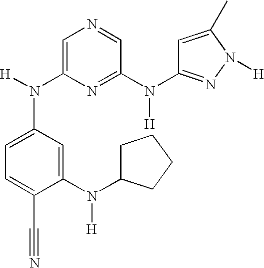 | 2-(cyclopentylamino)-4-(6-(5- methyl-1H-pyrazol-3- ylamino)pyrazin-2- ylamino)benzonitrile | 375.0 |
| 70 | 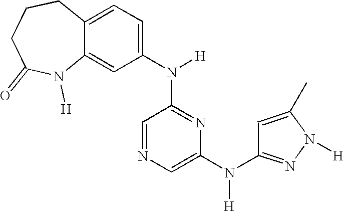 | 8-(6-(5-methyl-1H-pyrazol-3- ylamino)pyrazin-2- ylamino)octahydro-1H- benzo[b]azepin-2(3H)-one | 350.6 |
| 71 | 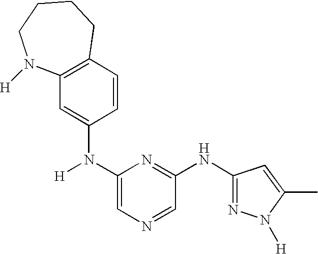 | N²-(5-methyl-1H-pyrazol-3-yl)-N⁶- (2,3,4,5-tetrahydro-1H- benzo[b]azepin-8-yl)pyrazine-2,6- diamine | 336.2 |
| 72 | 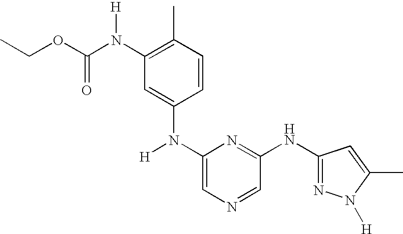 | ethyl 2-methyl-5-(6-(5-methyl-1H- pyrazol-3-ylamino)pyrazin-2- ylamino)phenylcarbamate | 368.4 |
| 73 | 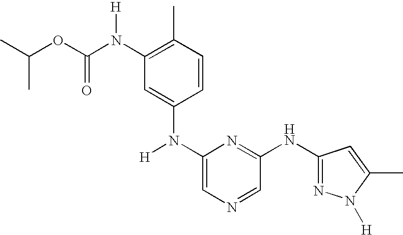 | isopropyl 2-methyl-5-(6-(5-methyl- 1H-pyrazol-3-ylamino)pyrazin-2- ylamino)phenylcarbamate | 382.4 |
| 74 | 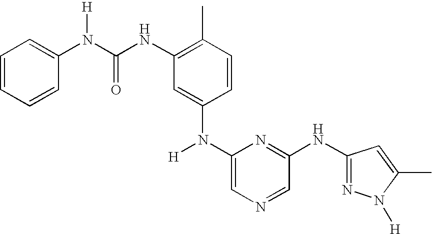 | 1-(2-methyl-5-(6-(5-methyl-1H- pyrazol-3-ylamino)pyrazin-2- ylamino)phenyl)-3-phenylurea | 415.4 |
| 75 | 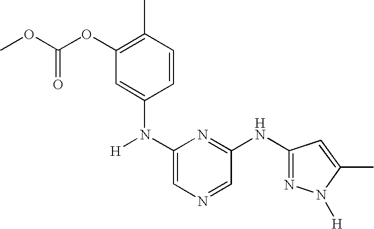 | methyl 2-methyl-5-(6-(5-methyl-1H- pyrazol-3-ylamino)pyrazin-2- ylamino)phenyl carbonate | 355.3 |
| 76 | 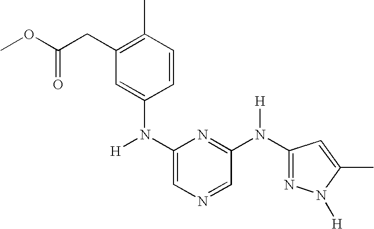 | methyl 2-(2-methyl-5-(6-(5-methyl- 1H-pyrazol-3-ylamino)pyrazin-2- ylamino)phenyl)acetate | 353.4 |
| 77 | 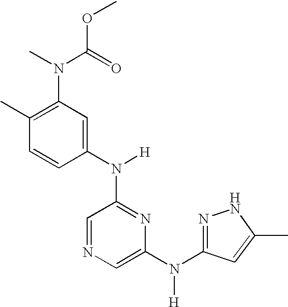 | methyl methyl(2-methyl-5-(6-(5- methyl-1H-pyrazol-3- ylamino)pyrazin-2- ylamino)phenyl)carbamate | 368.4 |
| 78 | 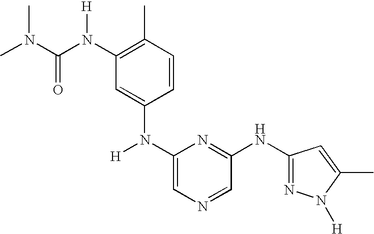 | 1,1-dimethyl-3-(2-methyl-5-(6-(5- methyl-1H-pyrazol-3- ylamino)pyrazin-2- ylamino)phenyl)urea | 367.4 |
| 79 | 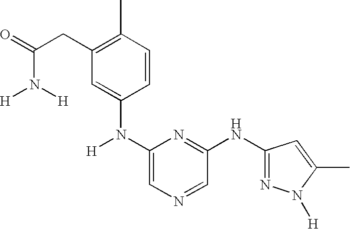 | 2-(2-methyl-5-(6-(5-methyl-1H- pyrazol-3-ylamino)pyrazin-2- ylamino)phenyl)acetamide | 338.4 |
| 80 | 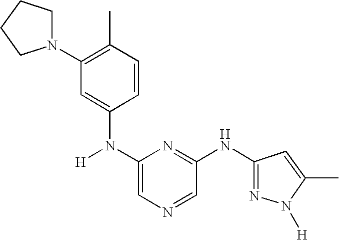 | N²-(5-methyl-1H-pyrazol-3-yl)-N⁶- (4-methyl-3-(pyrrolidin-1- yl)phenyl)pyrazine-2,6-diamine | 350.4 |
| 81 | 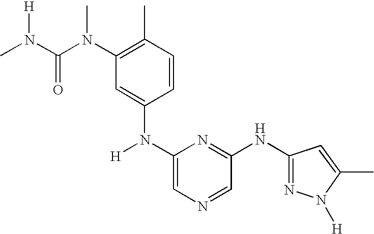 | 1,3-dimethyl-1-(2-methyl-5-(6-(5- methyl-1H-pyrazol-3- ylamino)pyrazin-2- ylamino)phenyl)urea | 367.4 |
| 82 | 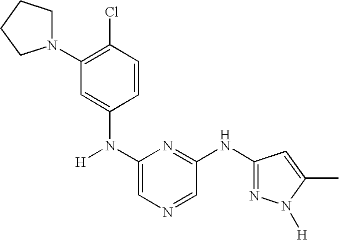 | N²-(4-chloro-3-(pyrrolidin-1- yl)phenyl)-N⁶-(5-methyl-1H- pyrazol-3-yl)pyrazine-2,6-diamine | 370.8 |
| 83 | 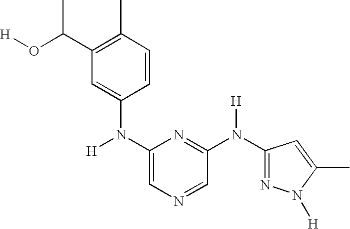 | 1-(2-methyl-5-(6-(5-methyl-1H- pyrazol-3-ylamino)pyrazin-2- ylamino)phenyl)ethanol | 325.3 |
| 84 | 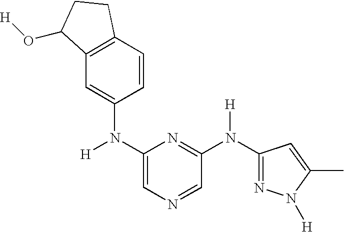 | 6-(6-(5-methyl-1H-pyrazol-3- ylamino)pyrazin-2-ylamino)-2,3- dihydro-1H-inden-1-ol | 323.3 |
| 85 | 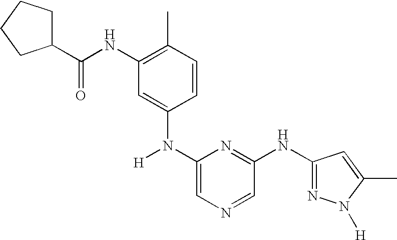 | N-(2-methyl-5-(6-(5-methyl-1H- pyrazol-3-ylamino)pyrazin-2- ylamino)phenyl)cyclopentanecarbox amide | 392.4 |
| 86 | 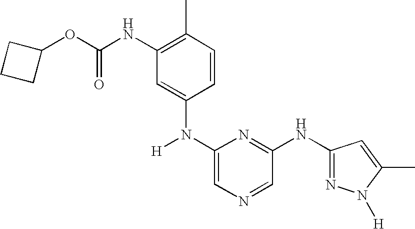 | cyclobutyl 2-methyl-5-(6-(5-methyl- 1H-pyrazol-3-ylamino)pyrazin-2- ylamino)phenylcarbamate | 394.4 |
| 87 | 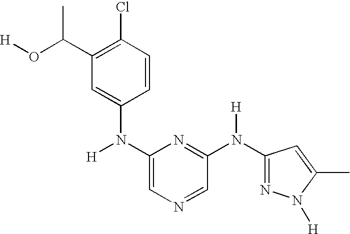 | 1-(2-chloro-5-(6-(5-methyl-1H- pyrazol-3-ylamino)pyrazin-2- ylamino)phenyl)ethanol | 345.8 |
| 88 | 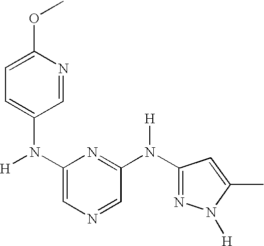 | N²-(6-methoxypyridin-3-yl)-N⁶-(5- methyl-1H-pyrazol-3-yl)pyrazine- 2,6-diamine | 298.3 |
| 89 | 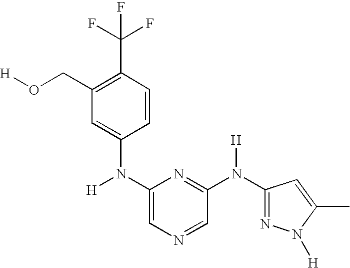 | (5-(6-(5-methyl-1H-pyrazol-3- ylamino)pyrazin-2-ylamino)-2- (trifluoromethyl)phenyl)methanol | 365.3 |
| 90 | 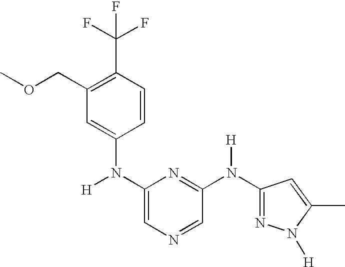 | N²-(3-(methoxymethyl)-4- (trifluoromethyl)phenyl)-N⁶-(5- methyl-1H-pyrazol-3-yl)pyrazine- 2,6-diamine | 379.3 |
| 91 | 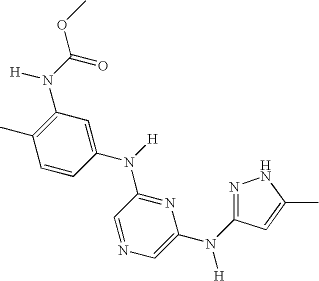 | methyl 2-methyl-5-(6-(5-methyl-1H- pyrazol-3-ylamino)pyrazin-2- ylamino)phenylcarbamate | 354.2 |
| 92 | 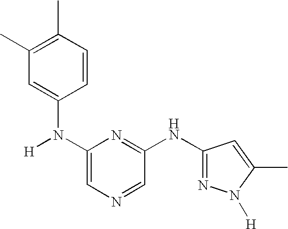 | N²-(3,4-dimethylphenyl)-N⁶-(5- methyl-1H-pyrazol-3-yl)pyrazine- 2,6-diamine | 295.3 |
| 93 | 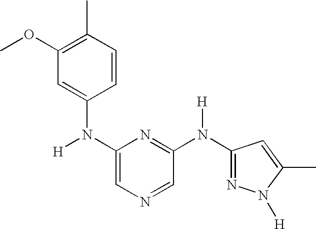 | N²-(3-methoxy-4-methylphenyl)-N⁶- (5-methyl-1H-pyrazol-3-yl)pyrazine- 2,6-diamine | 311.3 |
| 94 | 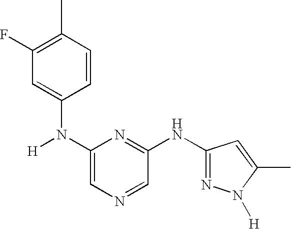 | N²-(3-fluoro-4-methylphenyl)-N⁶- (5-methyl-1H-pyrazol-3-yl)pyrazine- 2,6-diamine | 299.3 |
| 95 | 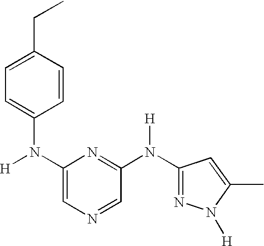 | N²-(4-ethylphenyl)-N⁶-(5-methyl- 1H-pyrazol-3-yl)pyrazine-2,6- diamine | 295.3 |
| 96 | 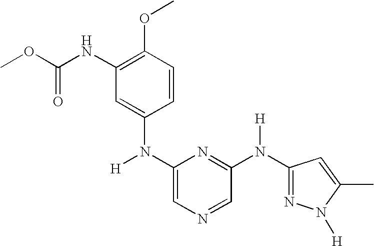 | methyl 2-methoxy-5-(6-(5-methyl- 1H-pyrazol-3-ylamino)pyrazin-2- ylamino)phenylcarbamate | 370.2 |
| 97 | 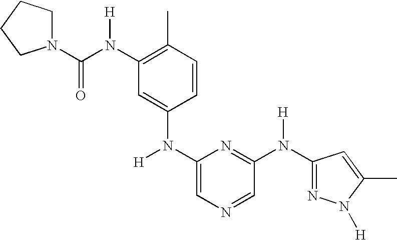 | N-(2-methyl-5-(6-(5-methyl-1H- pyrazol-3-ylamino)pyrazin-2- ylamino)phenyl)pyrrolidine-1- carboxamide | 393.3 |
| 98 | 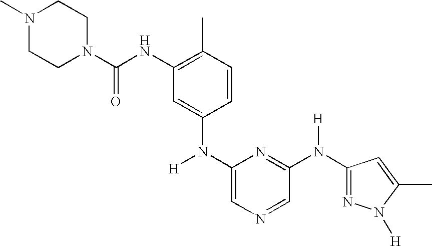 | 4-methyl-N-(2-methyl-5-(6-(5- methyl-1H-pyrazol-3- ylamino)pyrazin-2- ylamino)phenyl)piperazine-1- carboxamide | 422.2 |
| 99 | 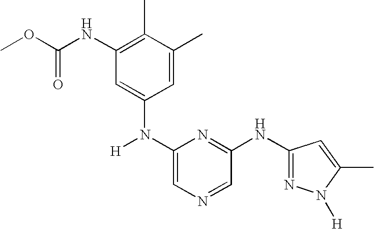 | methyl 2,3-dimethyl-5-(6-(5-methyl- 1H-pyrazol-3-ylamino)pyrazin-2- ylamino)phenylcarbamate | 368.3 |
| 100 | 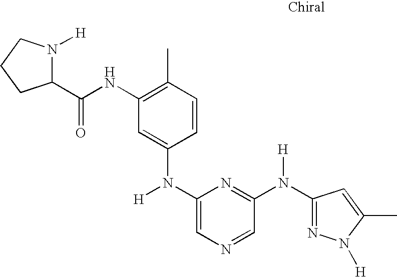 | (R)-N-(2-methyl-5-(6-(5-methyl-1H- pyrazol-3-ylamino)pyrazin-2- ylamino)phenyl)pyrrolidine-2- carboxamide | 393.3 |
| 101 | 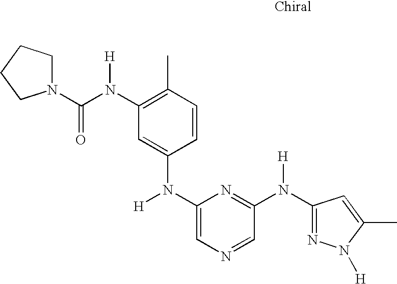 | (S)-N-(2-methyl-5-(6-(5-methyl-1H- pyrazol-3-ylamino)pyrazin-2- carboxamide | 393.3 |
| 102 | 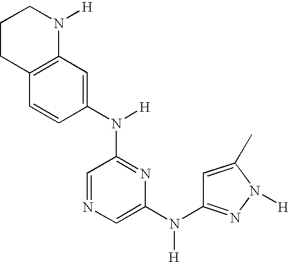 | N²-(5-methyl-1H-pyrazol-3-yl)-N⁶- (1,2,3,4-tetrahydroquinolin-7- yl)pyrazine-2,6-diamine | 322.5 |
| 103 | 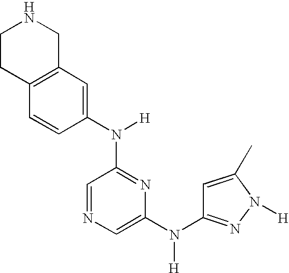 | N²-(5-methyl-1H-pyrazol-3-yl)-N⁶- (1,2,3,4-tetrahydroisoquinolin-7- yl)pyrazine-2,6-diamine | 322.5 |
| 104 | 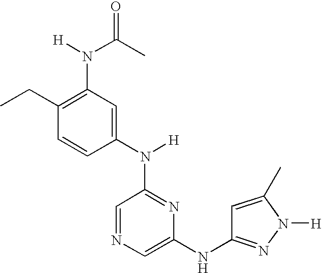 | N-(2-ethyl-5-(6-(5-methyl-1H- pyrazol-3-ylamino)pyrazin-2- ylamino)phenyl)acetamide | 352.3 |
| 105 | 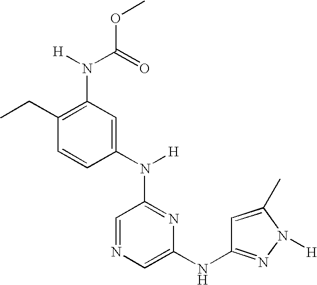 | methyl 2-ethyl-5-(6-(5-methyl-1H- pyrazol-3-ylamino)pyrazin-2- ylamino)phenylcarbamate | 368.1 |
| 106 | 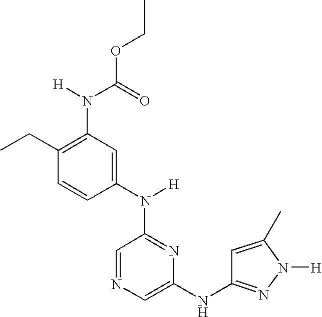 | ethyl 2-ethyl-5-(6-(5-methyl-1H- pyrazol-3-ylamino)pyrazin-2- ylamino)phenylcarbamate | 382.6 |
| 107 | 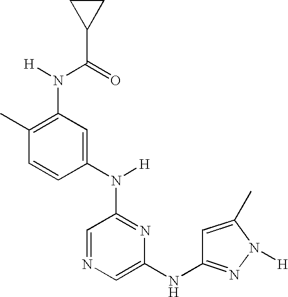 | N-(2-methyl-5-(6-(5-methyl-1H- pyrazol-3-ylamino)pyrazin-2- ylamino)phenyl)cyclopropanecarbox amide | 364.5 |
| 108 | 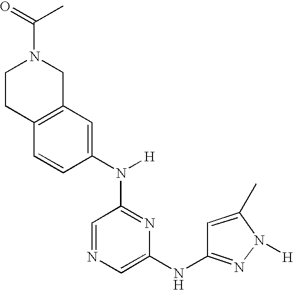 | 1-(7-(6-(5-methyl-1H-pyrazol-3- ylamino)pyrazin-2-ylamino)-3,4- dihydroisoquinolin-2(1H)- yl)ethanone | 364.6 |
| 109 | 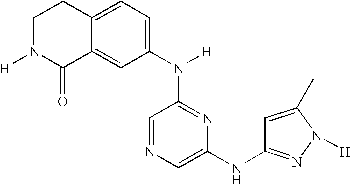 | N²-(5-methyl-1H-pyrazol-3-yl)-N⁶- (6-methylpyridin-3-yl)pyrazine-2,6- diamine | 336.2 |
| 110 | 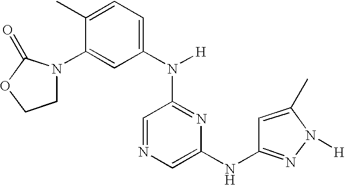 | 3-(2-methyl-5-(6-(5-methyl-1H- pyrazol-3-ylamino)pyrazin-2- ylamino)phenyl)oxazolidin-2-one | 365.8 |
| 111 | 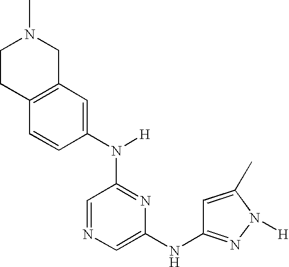 | N²-(2-methyl-1,2,3,4- tetrahydroisoquinolin-7-yl)-N⁶-(5- methyl-1H-pyrazol-3-yl)pyrazine- 2,6-diamine | 336.1 |
| 112 | 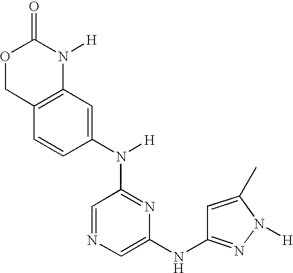 | 7-(6-(5-methyl-1H-pyrazol-3- ylamino)pyrazin-2-ylamino)-1H- benzo[d][1,3]oxazin-2(4H)-one | 338.4 |
| 113 | 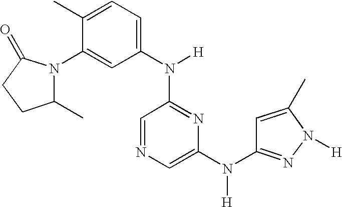 | N²-(2-methyl-1,2,3,4- tetrahydroisoquinolin-7-yl)-N⁶-(5- methyl-1H-pyrazol-3-yl)pyrazine- 2,6-diamine | 378.8 |
| 114 | 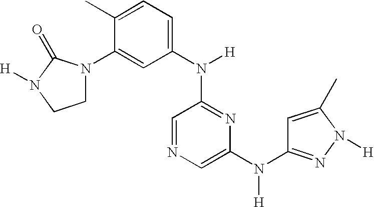 | 1-(2-methyl-5-(6-(5-methyl-1H- pyrazol-3-ylamino)pyrazin-2- ylamino)phenyl)imidazolidin-2-one | 365.3 |
| 115 | 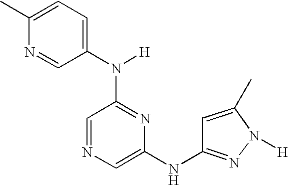 | N²-(5-methyl-1H-pyrazol-3-yl)-N⁶- (6-methylpyridin-3-yl)pyrazine-2,6- diamine | 282.2 |
| 116 | 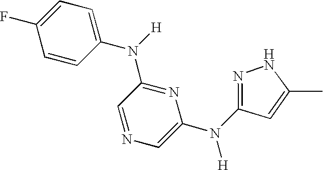 | N²-(4-fluorophenyl)-N⁶-(5-methyl- 1H-pyrazol-3-yl)pyrazine-2,6- diamine | 285.1 |
| 117 | 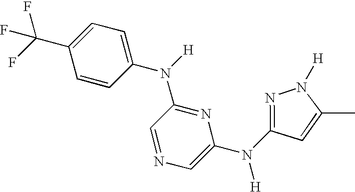 | N²-(5-methyl-1H-pyrazol-3-yl)-N⁶- (4-(trifluoromethyl)phenyl)pyrazine- 2,6-diamine | 335.4 |
| 118 | 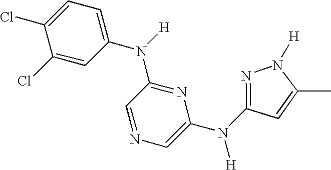 | N²-(3,4-dichlorophenyl)-N⁶-(5- methyl-1H-pyrazol-3-yl)pyrazine- 2,6-diamine | 335.3 |
| 119 | 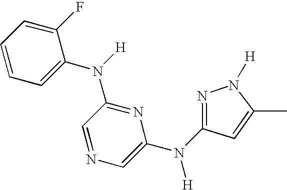 | N²-(2-fluorophenyl)-N⁶-(5-methyl- 1H-pyrazol-3-yl)pyrazine-2,6- diamine | 285.2 |
| 120 | 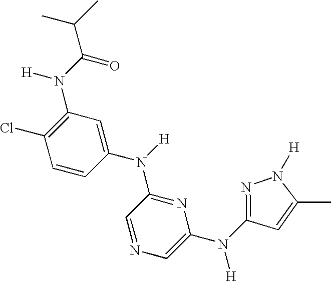 | N-(2-chloro-5-(6-(5-methyl-1H- pyrazol-3-ylamino)pyrazin-2- ylamino)phenyl)isobutyramide | 386.2 |
| 121 | 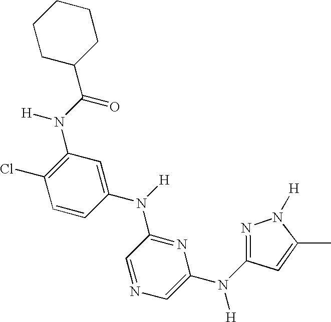 | N-(2-chloro-5-(6-(5-methyl-1H- pyrazol-3-ylamino)pyrazin-2- ylamino)phenyl)cyclohexanecar- boxamide | 426.3 |
| 122 | 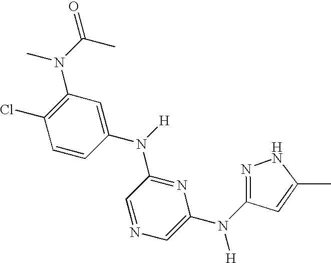 | N-(2-chloro-5-(6-(5-methyl-1H- pyrazol-3-ylamino)pyrazin-2- ylamino)phenyl)-N-methylacetamide | 372.3 |
| 123 | 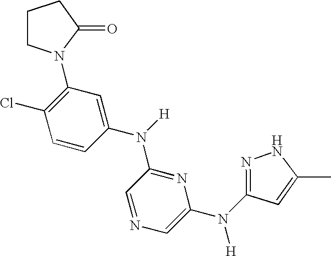 | 1-(2-chloro-5-(6-(5-methyl-1H- pyrazol-3-ylamino)pyrazin-2- ylamino)phenyl)pyrrolidin-2-one | 384.3 |
| 124 | 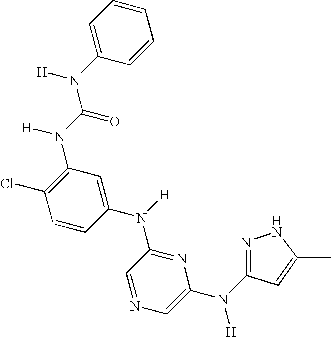 | 1-(2-chloro-5-(6-(5-methyl-1H- pyrazol-3-ylamino)pyrazin-2- ylamino)phenyl)-3-phenylurea | 435.3 |
| 125 | 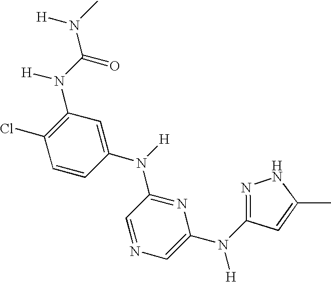 | 1-(2-chloro-5-(6-(5-methyl-1H- pyrazol-3-ylamino)pyrazin-2- ylamino)phenyl)-3-methylurea | 373.3 |
| 126 | 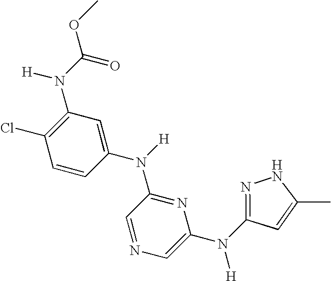 | methyl 2-chloro-5-(6-(5-methyl-1H- pyrazol-3-ylamino)pyrazin-2- ylamino)phenylcarbamate | 374.3 |
| 127 | 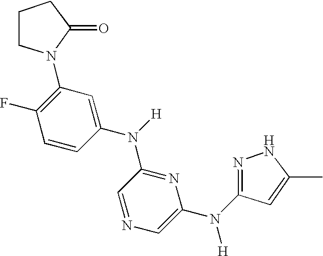 | 1-(2-fluoro-5-(6-(5-methyl-1H- pyrazol-3-ylamino)pyrazin-2- ylamino)phenyl)pyrrolidin-2-one | 368.3 |
| 128 | 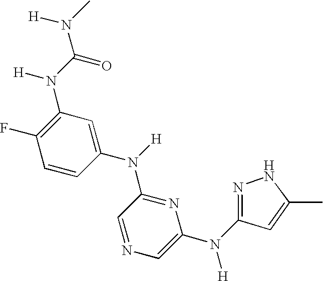 | 1-(2-fluoro-5-(6-(5-methyl-1H- pyrazol-3-ylamino)pyrazin-2- ylamino)phenyl)-3-methylurea | 357.5 |
| 129 | 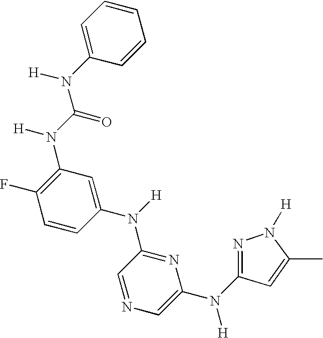 | 1-(2-fluoro-5-(6-(5-methyl-1H- pyrazol-3-ylamino)pyrazin-2- ylamino)phenyl)-3-phenylurea | 419.4 |
| 130 | 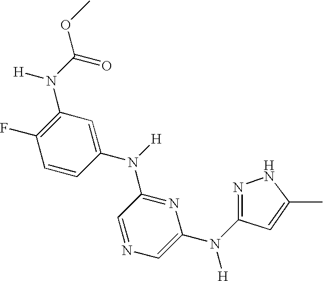 | methyl 2-fluoro-5-(6-(5-methyl-1H- pyrazol-3-ylamino)pyrazin-2- ylamino)phenylcarbamate | 358.4 |
| 131 | 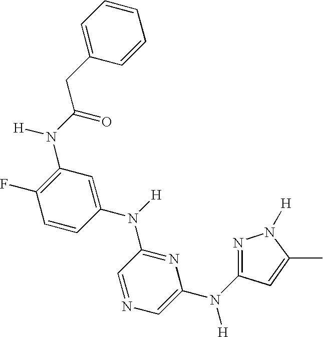 | N-(2-fluoro-5-(6-(5-methyl-1H- pyrazol-3-ylamino)pyrazin-2- ylamino)phenyl)-2-phenylacetamide | 418.5 |
| 132 | 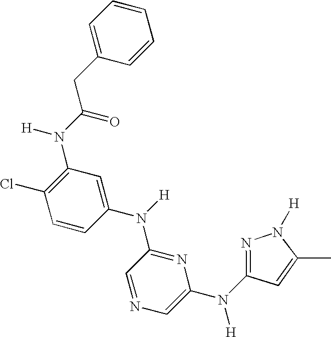 | N-(2-chloro-5-(6-(5-methyl-1H- pyrazol-3-ylamino)pyrazin-2- ylamino)phenyl)-2-phenylacetamide | 434.3 |
| 133 | 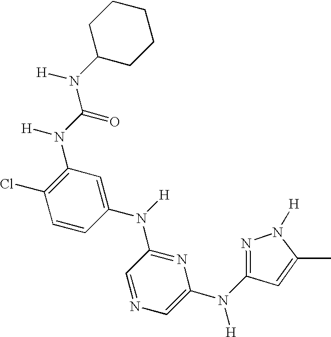 | 1-(2-chloro-5-(6-(5-methyl-1H- pyrazol-3-ylamino)pyrazin-2- ylamino)phenyl)-3-cyclohexylurea | 441.6 |
| 134 | 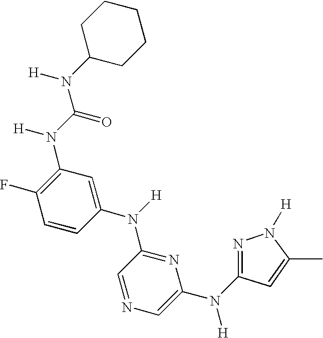 | 1-cyclohexyl-3-(2-fluoro-5-(6-(5- methyl-1H-pyrazol-3- ylamino)pyrazin-2- ylamino)phenyl)urea | 425.4 |
| 135 | 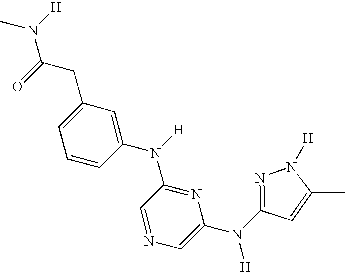 | N-methyl-2-(3-(6-(5-methyl-1H- pyrazol-3-ylamino)pyrazin-2- ylamino)phenyl)acetamide | 338.4 |
| 136 | 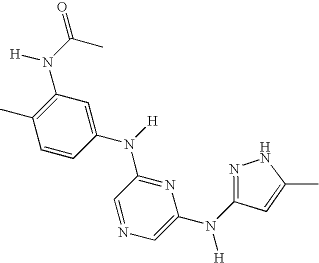 | N-(2-methyl-5-(6-(5-methyl-1H- pyrazol-3-ylamino)pyrazin-2- ylamino)phenyl)acetamide | 338.5 |
| 137 | 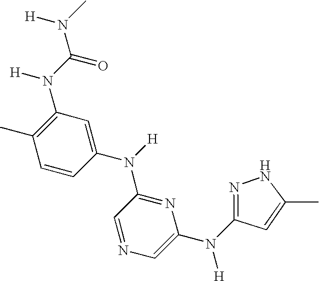 | 1-methyl-3-(2-methyl-5-(6-(5- methyl-1H-pyrazol-3- ylamino)pyrazin-2- ylamino)phenyl)urea | 353.4 |
| 138 | 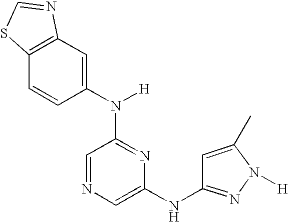 | N²-(benzo[d]thiazol-5-yl)-N⁶-(5- methyl-1H-pyrazol-3-yl)pyrazine- 2,6-diamine | 324.5 |
| 139 | 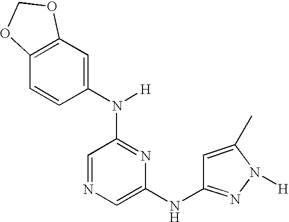 | N²-(benzo[d][1,3]dioxol-5-yl)-N⁶- (5-methyl-1H-pyrazol-3-yl)pyrazine- 2,6-diamine | 311.4 |
| 140 | 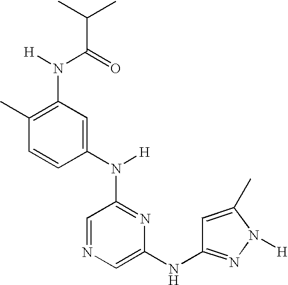 | N-(2-methyl-5-(6-(5-methyl-1H- pyrazol-3-ylamino)pyrazin-2- ylamino)phenyl)isobutyramide | 366.5 |
| 141 | 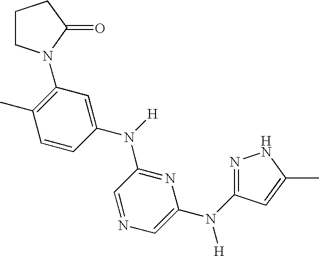 | 1-(2-methyl-5-(6-(5-methyl-1H- pyrazol-3-ylamino)pyrazin-2- ylamino)phenyl)pyrrolidin-2-one | 364.6 |
| 142 | 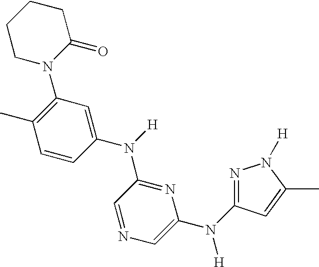 | 1-(2-methyl-5-(6-(5-methyl-1H- pyrazol-3-ylamino)pyrazin-2- ylamino)phenyl)piperidin-2-one | 378.6 |
| 143 | 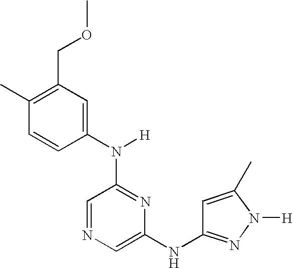 | N²-(3-(methoxymethyl)-4- methylphenyl)-N⁶-(5-methyl-1H- pyrazol-3-yl)pyrazine-2,6-diamine | 325.5 |
| 144 | 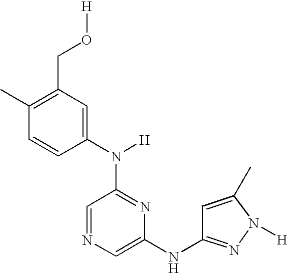 | (2-methyl-5-(6-(5-methyl-1H- pyrazol-3-ylamino)pyrazin-2- ylamino)phenyl)methanol | 311.5 |
| 145 | 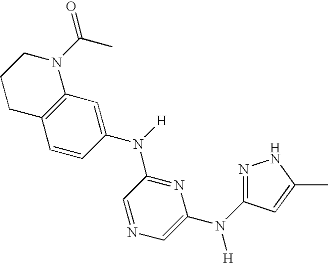 | 1-(7-(6-(5 -methyl-1H-pyrazol-3- ylamino)pyrazin-2-ylamino)-3,4- dihydroquinolin-1(2H)-yl)ethanone | 364.5 |
| 146 | 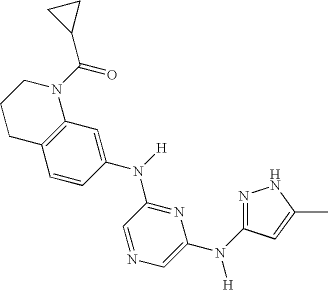 | cyclopropyl(7-(6-(5-methyl-1H- pyrazol-3-ylamino)pyrazin-2- ylamino)-3,4-dihydroquinolin-1(2H)- yl)methanone | 390.6 |
| 147 | 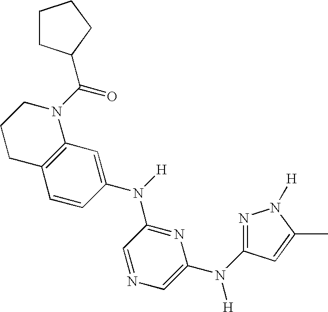 | cyclopentyl(7-(6-(5-methyl-1H- pyrazol-3-ylamino)pyrazin-2- ylamino)-3,4-dihydroquinolin-1(2H)- yl)methanone | 418.8 |
| 148 | 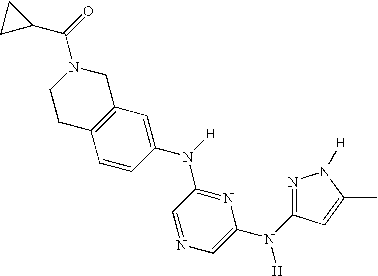 | cyclopropyl(7-(6-(5-methyl-1H- pyrazol-3-ylamino)pyrazin-2- ylamino)-3,4-dihydroisoquinolin- 2(1H)-yl)methanone | 390.6 |
| 149 | 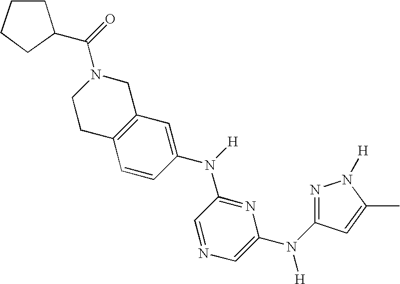 | cyclopentyl(7-(6-(5-methyl-1H- pyrazol-3-ylamino)pyrazin-2- ylamino)-3,4-dihydroisoquinolin- 2(1H)-yl)methanone | 418.8 |
| 150 | 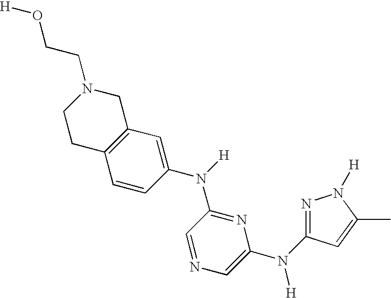 | 2-(7-(6-(5-methyl-1H-pyrazol-3- ylamino)pyrazin-2-ylamino)-3,4- dihydroisoquinolin-2(1H)-yl)ethanol | 366.5 |
| 151 | 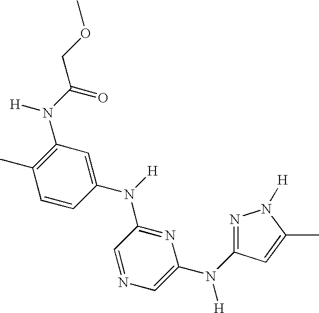 | 2-methoxy-N-(2-methyl-5-(6-(5- methyl-1H-pyrazol-3- ylamino)phenyl)acetamide | 368.5 |
| 152 | 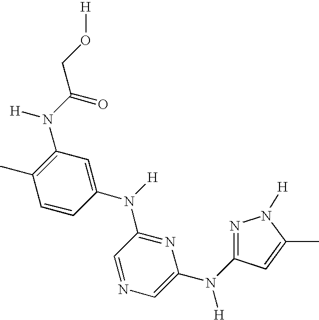 | 2-hydroxy-N-(2-methyl-5-(6-(5- methyl-1H-pyrazol-3- ylamino)pyrazin-2- ylamino)phenyl)acetamide | 354.5 |
| 153 | 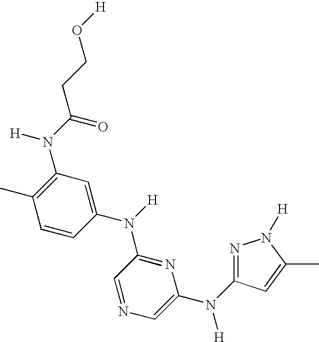 | 3-hydroxy-N-(2-methyl-5-(6-(5- methyl-1H-pyrazol-3- ylamino)pyrazin-2- ylamino)phenyl)propanamide | 368.5 |
| 154 | 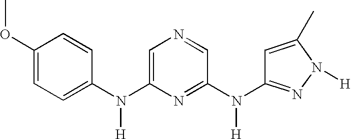 | N²-(4-methoxyphenyl)-N⁶-(5- methyl-1H-pyrazol-3-yl)pyrazine- 2,6-diamine | 297.15 |
| 155 | 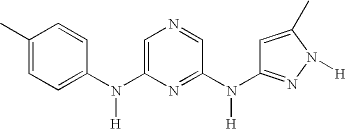 | N²-(5-methyl-1H-pyrazol-3-yl)-N⁶- p-tolylpyrazine-2,6-diamine | 281.2 |
| 156 | 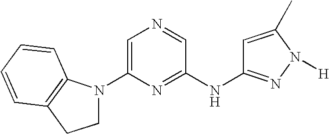 | 6-(indolin-1-yl)-N-(5-methyl-1H- pyrazol-3-yl)pyrazin-2-amine | 293.1 |
| 157 | 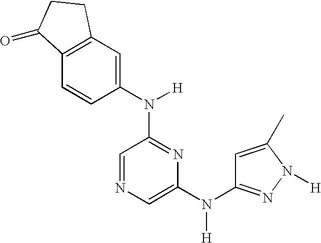 | 5-(6-(5-methyl-1H-pyrazol-3- ylamino)pyrazin-2-ylamino)-2,3- dihydro-1H-inden-1-one | 321.1 |
| 158 | 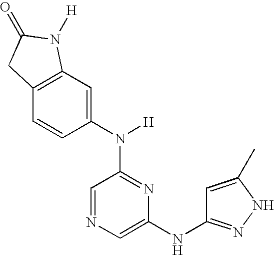 | 6-(6-(5-methyl-1H-pyrazol-3- ylamino)pyrazin-2-ylamino)indolin- 2-one | 322.2 |
| 159 | 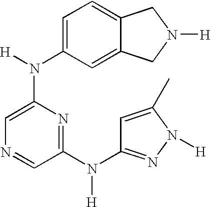 | N²-(isoindolin-5-yl)-N⁶-(5-methyl- 1H-pyrazol-3-yl)pyrazine-2,6- diamine | 308.3 |
| 160 | 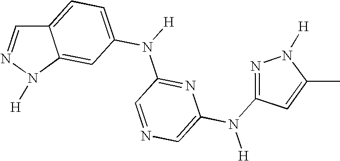 | N²-(1H-indazol-6-yl)-N⁶-(5-methyl- 1H-pyrazol-3-yl)pyrazine-2,6- diamine | 307.2 |
| 161 | 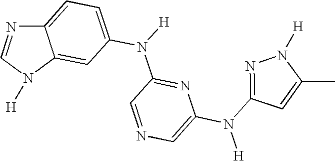 | 2-(1H-benzo[d]imidazol-6-yl)-N⁶- (5-methyl-1H-pyrazol-3-yl)pyrazine- 2,6-diamine | 307.2 |
| 162 | 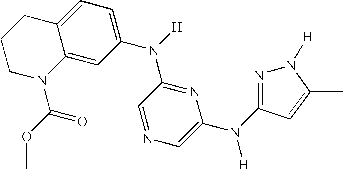 | methyl 7-(6-(5-methyl-1H-pyrazol-3- ylamino)pyrazin-2-ylamino)-3,4- dihydroquinoline-1(2H)-carboxylate | 380.0 |
| 163 | 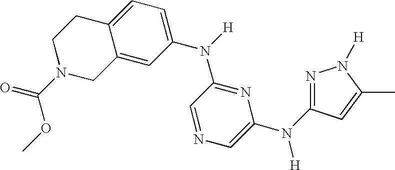 | methyl 7-(6-(5-methyl-1H-pyrazol-3- ylamino)pyrazin-2-ylamino)-3,4- dihydroisoquinoline-2(1H)- carboxylate | 380.2 |
| 164 | 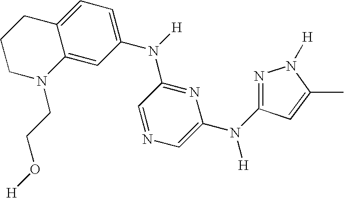 | 2-(7-(6-(5-methyl-1H-pyrazol-3- ylamino)pyrazin-2-ylamino)-3,4- dihydroquinolin-1(2H)-yl)ethanol | 366.3 |
| 165 | 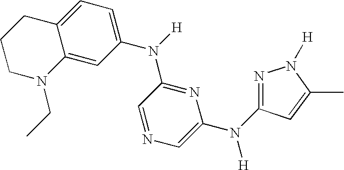 | 2-(1-ethyl-1,2,3,4- tetrahydroquinolin-7-yl)-N⁶-(5- methyl-1H-pyrazol-3-yl)pyrazine- 2,6-diatnine | 350.3 |
| 166 | 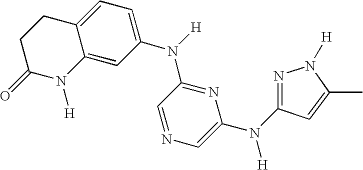 | 7-(6-(5-methyl-1H-pyrazol-3- ylamino)pyrazin-2-ylamino)-3,4- dihydroquinolin-2(1H)-one | 336.2 |
| 167 | 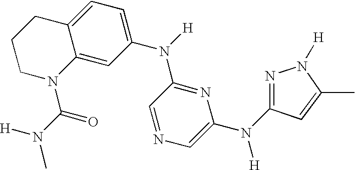 | N-methyl-7-(6-(5-methyl-1H- pyrazol-3-ylamino)pyrazin-2- ylamino)-3,4-dihydroquinoline- 1(2H)-carboxamide | 379.2 |
| 168 | 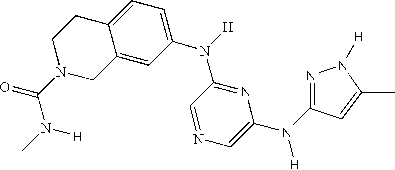 | N-methyl-7-(6-(5-methyl-1H- pyrazol-3-ylamino)pyrazin-2- ylamino)-3,4-dihydroisoquinoline- 2(1H)-carboxamide | 379.2 |
| 169 | 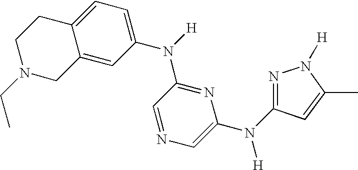 | N²-(2-ethyl-1,2,3,4- tetrahydroisoquinolin-7-yl)-N⁶-(5- methyl-1H-pyrazol-3-yl)pyrazine- 2,6-diamine | 350.1 |
| 170 | 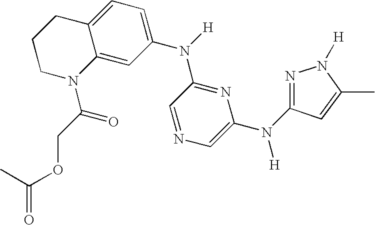 | 2-(7-(6-(5-methyl-1H-pyrazol-3- ylamino)pyrazin-2-ylamino)-3,4- dihydroquinolin-1(2H)-yl)-2- oxoethyl acetate | 422.1 |
| 171 | 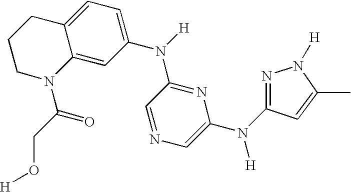 | 2-hydroxy-1-(7-(6-(5-methyl-1H- pyrazol-3-ylamino)pyrazin-2- ylamino)-3,4-dihydroquinolin-1(2H)- yl)ethanone | 380.1 |
| 172 | 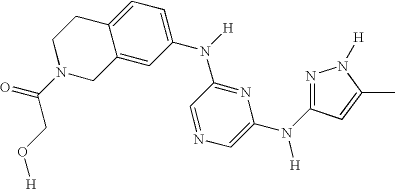 | 2-hydroxy-1-(7-(6-(5-methyl-1H- pyrazol-3-ylamino)pyrazin-2- ylamino)-3,4-dihydroisoquinolin- 2(1H)-yl)ethanone | 380.1 |
| 173 | 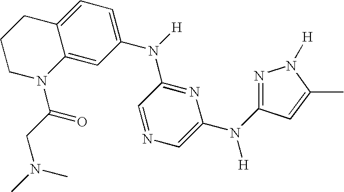 | 2-(dimethylamino)-1-(7-(6-(5- methyl-1H-pyrazol-3- ylamino)pyrazin-2-ylamino)-3,4- dihydroquinolin-1(2H)-yl)ethanone | 407.1 |
| 174 | 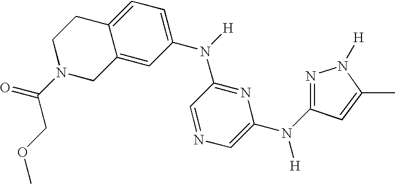 | 2-methoxy-1-(7-(6-(5-methyl-1H- pyrazol-3-ylamino)pyrazin-2- ylamino)-3,4-dihydroisoquinolin- 2(1H)-yl)ethanone | 394.2 |
| 175 | 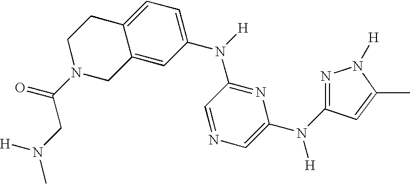 | 1-(7-(6-(5-methyl-1H-pyrazol-3- ylamino)pyrazin-2-ylamino)-3,4- dihydroisoquinolin-2(1H)-yl)-2- (methylamino)ethanone | 393.3 |
| 176 | 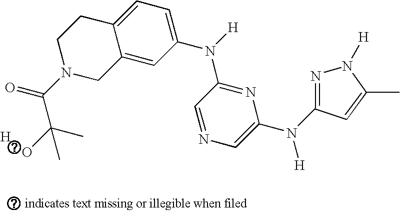 | 2-hydroxy-2-methyl-1-(7-(6-(5- methyl-1H-pyrazol-3- ylamino)pyrazin-2-ylamino)-3,4- dihydroisoquinolin-2(1H)-yl)propan- 1-one | 408.3 |
| 177 | 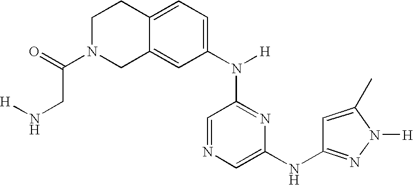 | 2-amino-1-(7-(6-(5-methyl-1H- pyrazol-3-ylamino)pyrazin-2- ylamino)-3,4-dihydroisoquinolin- 2(1H)-yl)ethanone | 379.2 |
| 178 | 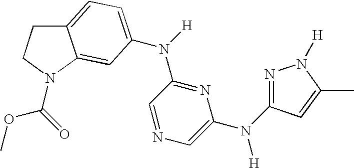 | methyl 6-(6-(5-methyl-1H-pyrazol-3- ylamino)pyrazin-2-ylamino)indoline- 1-carboxylate | 366.2 |
| 179 | 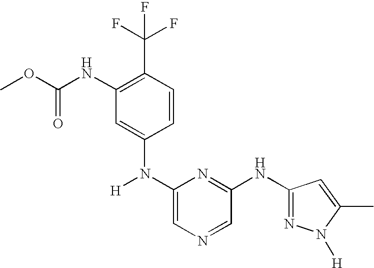 | methyl 5-(6-(5-methyl-1H-pyrazol-3- ylamino)pyrazin-2-ylamino)-2- (trifluoromethyl)phenylcarbamate | 408.1 |
| 180 | 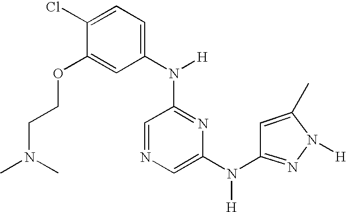 | N²-(4-chloro-3-(2- (dimethylamino)ethoxy)phenyl)-N⁶- (5-methyl-1H-pyrazo1-3-yl)pyrazine- 2,6-diamine | 387.1 |
| 181 | 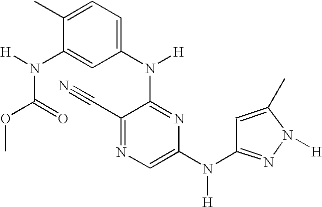 | 1-(2-methyl-5-(6-(5-methyl-1H- pyrazol-3-ylamino)pyrazin-2- ylamino)phenyl)imidazolidin-2-one | 379.5 |
| 182 | 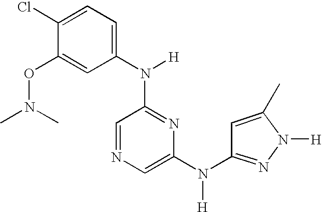 | 2-chloro-N,N-dimethyl-5-(6-(5- methyl-1H-pyrazol-3- ylamino)pyrazin-2- ylamino)benzamide | 372.1 |
| 183 | 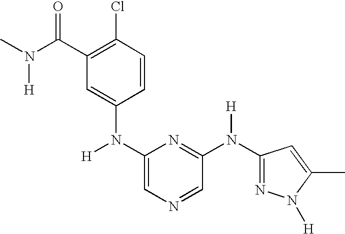 | 2-chloro-N-methyl-5-(6-(5-methyl- 1H-pyrazol-3-ylamino)pyrazin-2- ylamino)benzamide | 358.1 |
| 184 | 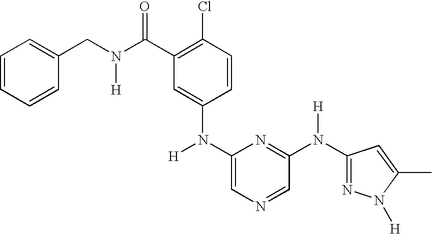 | N-benzyl-2-chloro-5-(6-(5-methyl- 1H-pyrazol-3-ylamino)pyrazin-2- ylamino)benzamide | 434.1 |
| 185 | 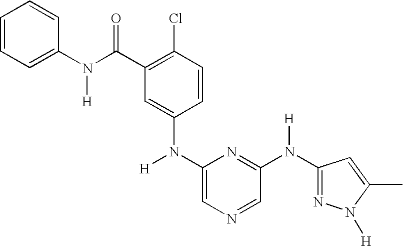 | 2-chloro-5-(6-(5-methyl-1H-pyrazol- 3-ylamino)pyrazin-2-ylamino)-N- phenylbenzamide | 420.2 |
| 186 | 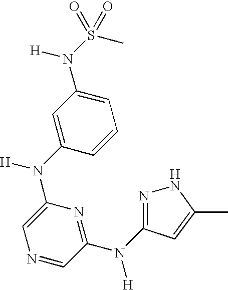 | N-(3-(6-(5-methyl-1H-pyrazol-3- ylamino)pyrazin-2- ylamino)phenyl)methanesulfonamide | 360.15 |
| 187 | 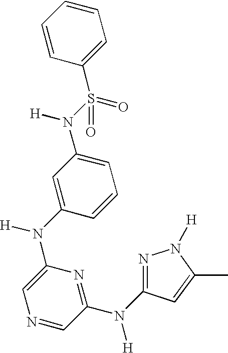 | N-(3-(6-(5-methyl-1H-pyrazol-3- ylamino)pyrazin-2- ylamino)phenyl)benzenesulfonamide | 422.15 |
| 188 | 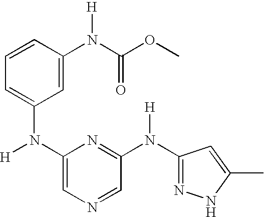 | methyl 3-(6-(5-methyl-1H-pyrazol-3- ylamino)pyrazin-2- ylamino)phenylcarbamate | 339.9 |
| 189 | 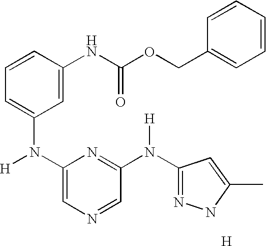 | benzyl 3-(6-(5-methyl-1H-pyrazol-3- ylamino)pyrazin-2- ylamino)phenylcarbamate | 416.1 |
| 190 | 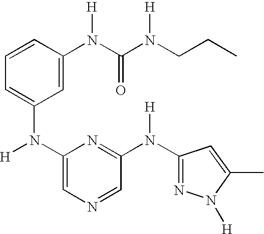 | 1-(3-(6-(5-methyl-1H-pyrazol-3- ylamino)pyrazin-2-ylamino)phenyl)- 3-propylurea | 367.15 |
| 191 | 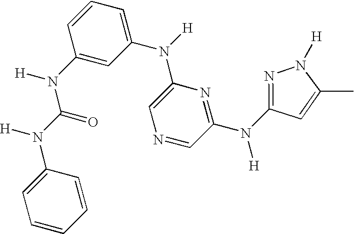 | 1-(3-(6-(5-methyl-1H-pyrazol-3- ylamino)pyrazin-2-ylamino)phenyl)- 3-phenylurea | 400.9 |
| 192 | 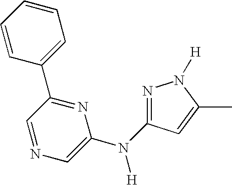 | N-(5-methyl-1H-pyrazol-3-yl)-6- phenylpyrazin-2-amine | 252.4 |
| 193 | 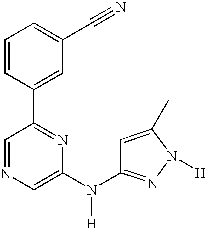 | 3-(6-(5-methyl-1H-pyrazol-3- ylamino)pyrazin-2-yl)benzonitrile | 277 |
| 194 | 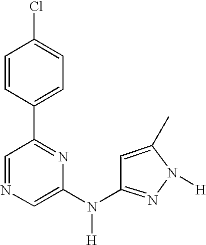 | 6-(4-chlorophenyl)-N-(5-methyl-1H- pyrazol-3-yl)pyrazin-2-amine | 286.4 |
| 195 | 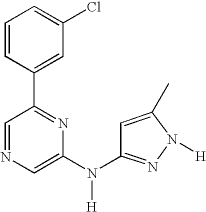 | 6-(3-chlorophenyl)-N-(5-methyl-1H- pyrazol-3-yl)pyrazin-2-amine | 286.1 |
| 196 | 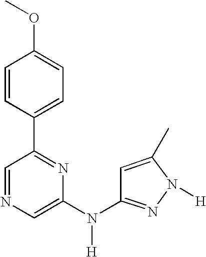 | 6-(4-methoxyphenyl)-N-(5-methyl- 1H-pyrazol-3-yl)pyrazin-2-amine | 282.5 |
| 197 | 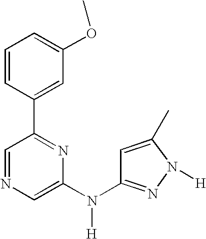 | 6-(3-methoxyphenyl)-N-(5-methyl- 1H-pyrazol-3-yl)pyrazin-2-amine | 282.5 |
| 198 | 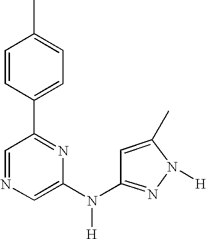 | N-(5-methyl-1H-pyrazol-3-yl)-6-p- tolylpyrazin-2-amine | 266.3 |
| 199 | 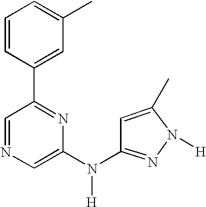 | N-(5-methyl-1H-pyrazol-3-yl)-6-m- tolylpyrazin-2-amine | 266.3 |
| 200 | 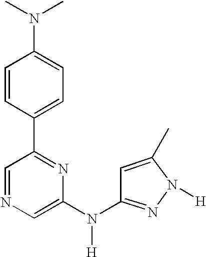 | 6-(4-(dimethylamino)phenyl)-N-(5- methyl-1H-pyrazol-3-yl)pyrazin-2- amine | 295.5 |
| 201 | 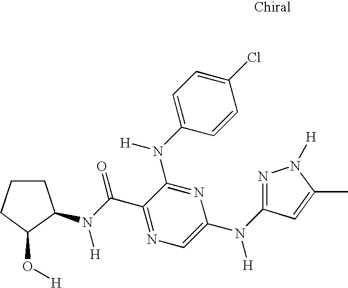 | 3-(4-chlorophenylamino)-N- ((1S,2S)-2-hydroxycyclopentyl)-5- (5-methyl-1H-pyrazol-3- ylamino)pyrazine-2-carboxamide | 428.0 |
| 202 | 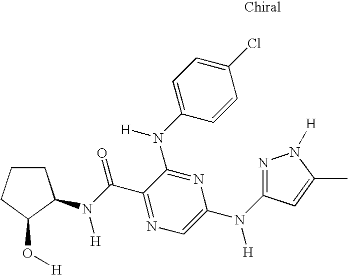 | 3-(4-chlorophenylamino)-N- ((1R,2R)-2-hydroxycyclopentyl)-5- (5-methyl-1H-pyrazol-3- ylamino)pyrazine-2-carboxamide | 428.0 |
| 203 | 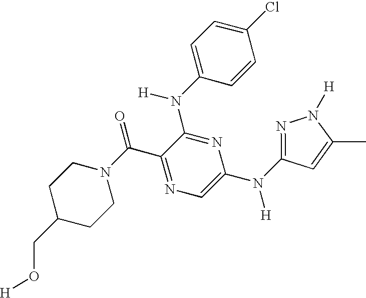 | (3-(4-chlorophenylamino)-5-(5- methyl-1H-pyrazol-3- ylamino)pyrazin-2-yl)(4- (hydroxymethyl)piperidin-1- yl)methanone | 442.4 |
| 204 | 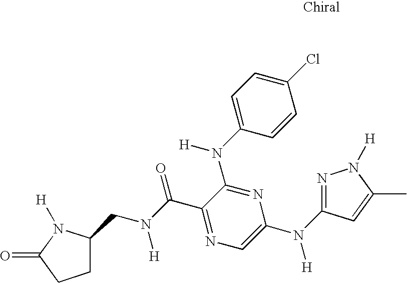 | (R)-3-(4-chlorophenylamino)-5-(5- methyl-1H-pyrazol-3-ylamino)-N- ((5-oxopyrrolidin-2- yl)methyl)pyrazine-2-carboxamide | 441.1 |
| 205 | 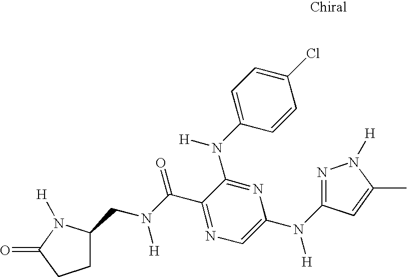 | (S)-3-(4-chlorophenylamino)-5-(5- methyl-1H-pyrazol-3-ylamino)-N- ((5-oxopyrrolidin-2- yl)methyl)pyrazine-2-carboxamide | 441.3 |
| 206 | 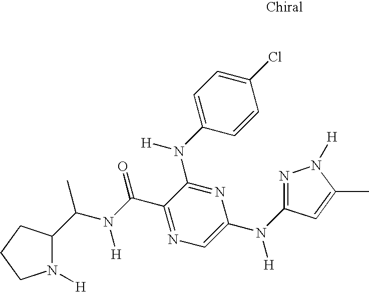 | (R)-3-(4-chlorophenylamino)-5-(5- methyl-1H-pyrazol-3-ylamino)-N- pyrrolidin-2-ylmethyl)pyrazine-2- carboxamide | 427.1 |
| 207 | 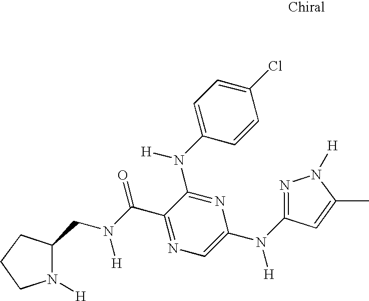 | (S)-3-(4-chlorophenylamino)-5-(5- methyl-1H-pyrazol-3-ylamino)-N- (pyrrolidin-2-ylmethyl)pyrazine-2- carboxamide | 427.1 |
| 208 | 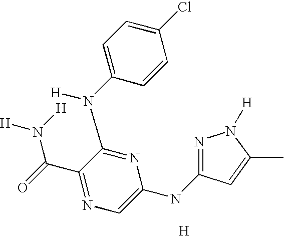 | 3-(4-chlorophenylamino)-5-(5- methyl-1H-pyrazol-3- ylamino)pyrazine-2-carboxamide | 344.4 |
| 209 | 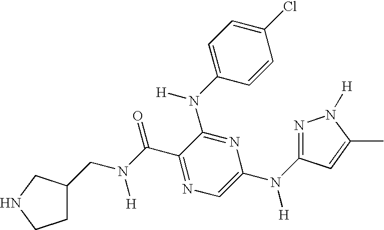 | 3-(4-chlorophenylamino)-5-(5- methyl-1H-pyrazol-3-ylamino)-N- (pyrrolidin-3-ylmethyl)pyrazine-2- carboxamide | 427.1 |
| 210 | 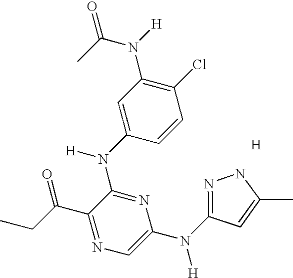 | N-(2-chloro-5-(6-(5-methyl-1H- pyrazol-3-ylamino)-3- propionylpyrazin-2- ylamino)phenyl)acetamide | 414.4 |
| 211 | 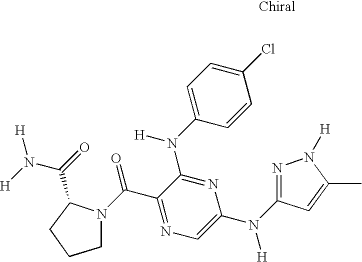 | (R)-1-(3-(4-chlorophenylamino)-5- (5-methyl-1H-pyrazol-3- ylamino)pyrazine-2- carbonyl)pyrrolidine-2-carboxamide | 441.5 |
| 212 | 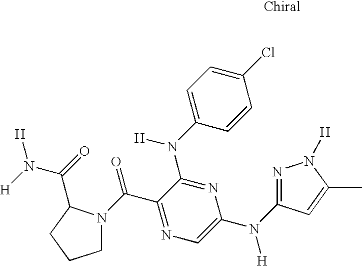 | (S)-1-(3-(4-chlorophenylamino)-5- (5-methyl-1H-pyrazol-3- ylamino)pyrazine-2- carbonyl)pyrrolidine-2-carboxamide | 441.4 |
| 213 | 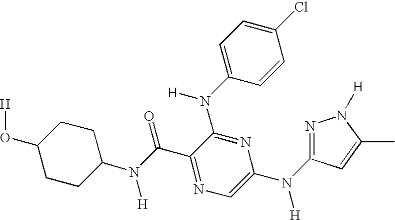 | 3-(4-chlorophenylamino)-N-((1s,4s)- 4-hydroxycyclohexyl)-5-(5-methyl- 1H-pyrazol-3-ylamino)pyrazine-2- carboxamide | 442.5 |
| 214 | 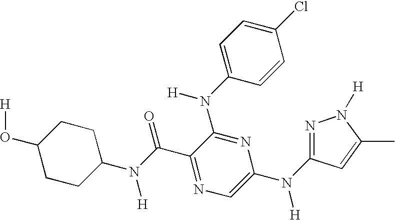 | 3-(4-chlorophenylamino)-N-((1r,4r)- 4-hydroxycyclohexyl)-5-(5-methyl- 1H-pyrazol-3-ylamino)pyrazine-2- carboxamide | 442.5 |
| 215 | 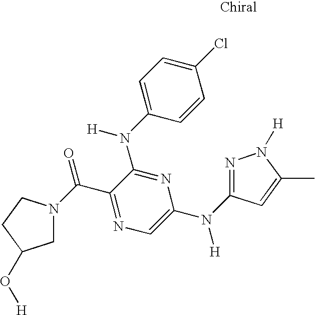 | (R)-(3-(4-chlorophenylamino)-5-(5- methyl-1H-pyrazol-3- ylamino)pyrazin-2-yl)(3- hydroxypyrrolidin-1-yl)methanone | 414.5 |
| 216 | 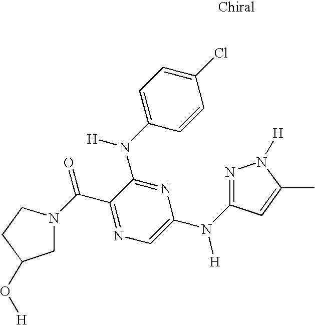 | (S)-(3-(4-chlorophenylamino)-5-(5- methyl-1H-pyrazol-3- ylamino)pyrazin-2-yl)(3- hydroxypyrrolidin-1-yl)methanone | 414.5 |
| 217 | 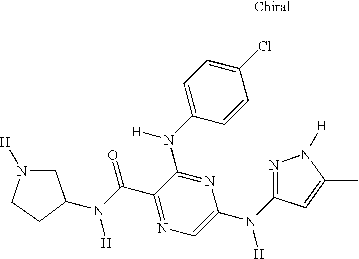 | (R)-3-(4-chlorophenylamino)-5-(5- methyl-1H-pyrazol-3-ylamino)-N- (pyrrolidin-3-yl)pyrazine-2- carboxamide | 413.1 |
| 218 | 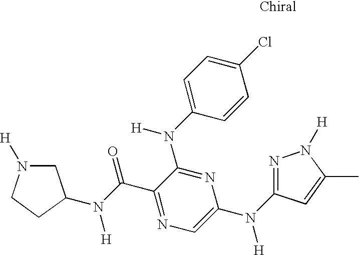 | (S)-3-(4-chlorophenylamino)-5-(5- methyl-1H-pyrazol-3-ylamino)-N- (pyrrolidin-3-yl)pyrazine-2- carboxamide | 413.1 |
| 219 | 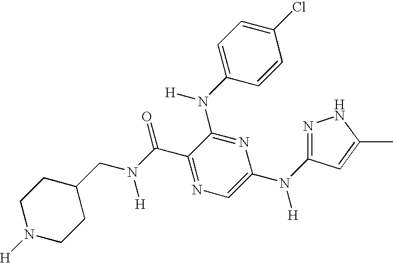 | 3-(4-chlorophenylamino)-5-(5- methyl-1H-pyrazol-3-ylamino)-N- (piperidin-4-ylmethyl)pyrazine-2- carboxamide | 441.1 |
| 220 | 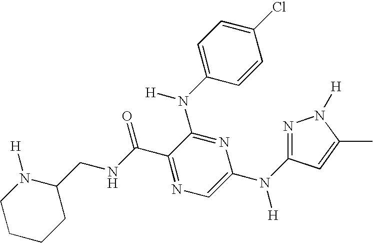 | 3-(4-chlorophenylamino)-5-(5- methyl-1H-pyrazol-3-ylamino)-N- (piperidin-2-ylmethyl)pyrazine-2- carboxamide | 441.1 |
| 221 | 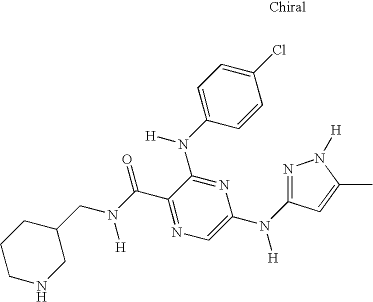 | (S)-3-(4-chlorophenylamino)-5-(5- methyl-1H-pyrazol-3-ylamino)-N- (piperidin-3-ylmethyl)pyrazine-2- carboxamide | 441.1 |
| 222 | 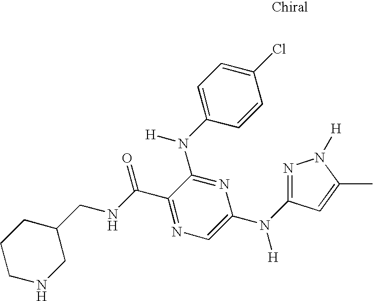 | (R)-3-(4-chlorophenylamino)-5-(5- methyl-1H-pyrazol-3-ylamino)-N- (piperidin-3-ylmethyl)pyrazine-2- carboxamide | 441.1 |
| 223 | 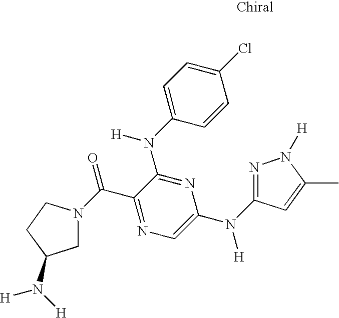 | (R)-(3-aminopyrrolidin-1-yl)(3-(4- chlorophenylamino)-5-(5-methyl- 1H-pyrazol-3-ylamino)pyrazin-2- yl)methanone | 413.1 |
| 224 | 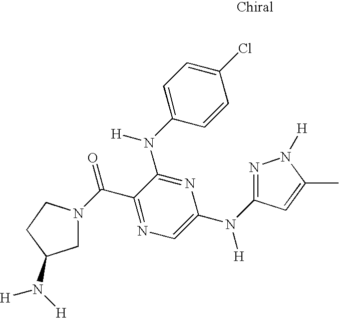 | (S)-(3-aminopyrrolidin-1-yl)(3-(4- chlorophenylamino)-5-(5-methyl- 1H-pyrazol-3-ylamino)pyrazin-2- yl)methanone | 413.1 |
| 225 | 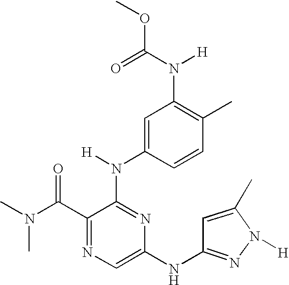 | methyl 5-(3-(dimethylcarbamoyl)-6- (5-methyl-1H-pyrazol-3- ylamino)pyrazin-2-ylamino)-2- methylphenylcarbamate | 425.1 |
| 226 | 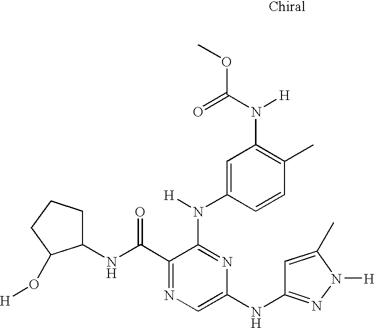 | methyl 5-(3-((1R,2R)-2- hydroxycyclopentylcarbamoyl)-6-(5- methyl-1H-pyrazol-3- ylamino)pyrazin-2-ylamino)-2- methylphenylcarbamate | 481.2 |
| 227 | 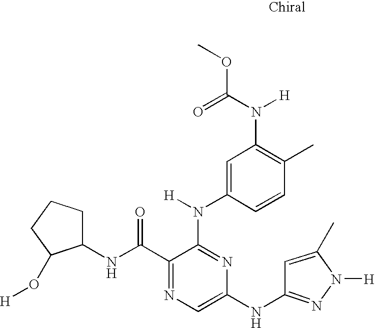 | methyl 5-(3-((1S,2S)-2- hydroxycyclopentylcarbamoyl)-6-(5- methyl-1H-pyrazol-3- ylamino)pyrazin-2-ylamino)-2- methylphenylcarbamate | 481.2 |
| 228 | 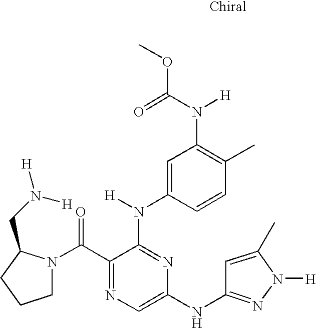 | (S)-methyl 5-(3-(2- (aminomethyl)pyrrolidine-1- carbonyl)-6-(5-methyl-1H-pyrazol-3- ylamino)pyrazin-2-ylamino)-2- methylphenylcarbamate | 480.4 |
| 229 | 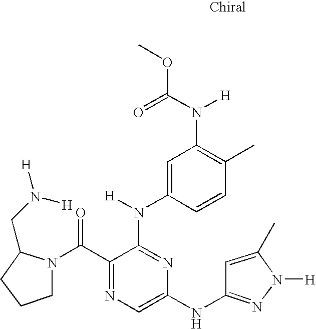 | (R)-methyl 5-(3-(2- (aminomethyl)pyrrolidine-1- carbonyl)-6-(5-methyl-1H-pyrazol-3- ylamino)pyrazin-2-ylamino)-2- methylphenylcarbamate | 480.4 |
| 230 | 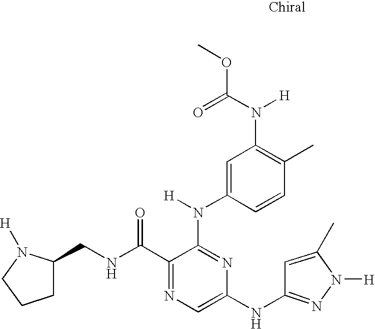 | (R)-methyl 2-methyl-5-(6-(5-methyl- 1H-pyrazol-3-ylamino)-3- (pyrrolidin-2- ylmethylcarbamoyl)pyrazin-2- ylamino)phenylcarbamate | 480.4 |
| 231 | 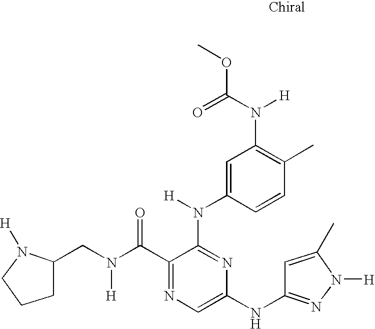 | (S)-methyl 2-methyl-5-(6-(5-methyl- 1H-pyrazol-3-ylamino)-3- (pyrrolidin-2- ylmethylcarbamoyl)pyrazin-2- ylamino)phenylcarbamate | 480.4 |
| 232 | 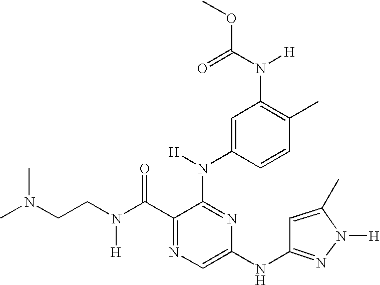 | methyl 5-(3-(2- (dimethylamino)ethylcarbamoyl)-6- (5-methyl-1H-pyrazol-3- ylamino)pyrazin-2-ylamino)-2- methylphenylcarbamate | 468.5 |
| 233 | 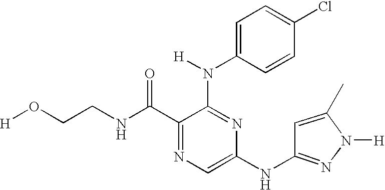 | 3-(4-chlorophenylamino)-N-(2- hydroxyethyl)-5-(5-methyl-1H- pyrazol-3-ylamino)pyrazine-2- carboxamide | 388.05 |
| 234 | 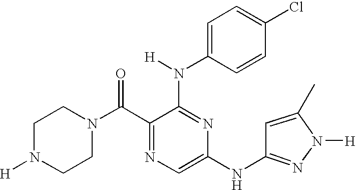 | (3-(4-chlorophenylamino)-5-(5- methyl-1H-pyrazol-3- ylamino)pyrazin-2-yl)(piperazin-1- yl)methanone | 413.1 |
| 235 | 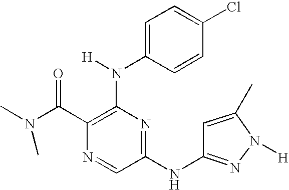 | 3-(4-chlorophenylamino)-N,N- dimethyl-5-(5-methyl-1H-pyrazol-3- ylamino)pyrazine-2-carboxamide | 372.15 |
| 236 | 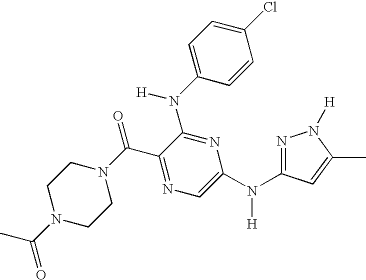 | 1-(4-(3-(4-chlorophenylamino)-5-(5- methyl-1H-pyrazol-3- ylamino)pyrazine-2- carbonyl)piperazin-1-yl)ethanone | 455.5 |
| 237 | 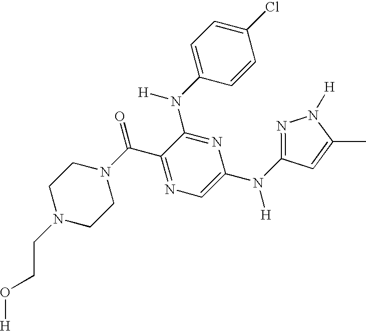 | (3-(4-chlorophenylamino)-5-(5- methyl-1H-pyrazol-3- ylamino)pyrazin-2-yl)(4-(2- hydroxyethyl)piperazin-1- yl)methanone | 457.2 |
| 238 | 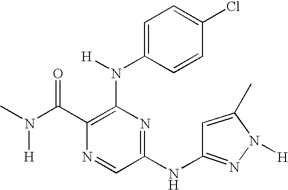 | 3-(4-chlorophenylamino)-N-methyl- 5-(5-methyl-1H-pyrazol-3- ylamino)pyrazine-2-carboxamide | 358.0 |
| 239 | 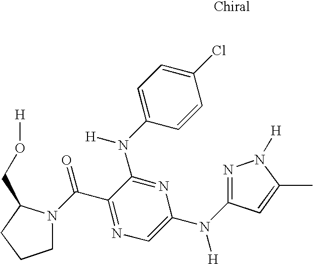 | (S)-(3-(4-chlorophenylamino)-5-(5- methyl-1H-pyrazol-3- ylamino)pyrazin-2-yl)(2- (hydroxymethyl)pyrrolidin-1- yl)methanone | 428.1 |
| 240 | 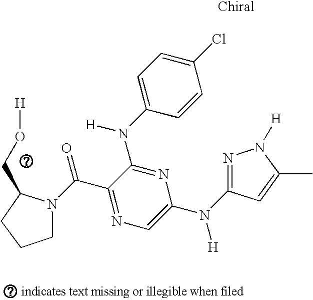 | (R)-(3-(4-chlorophenylamino)-5-(5- methyl-1H-pyrazol-3- ylamino)pyrazin-2-yl)(2- (hydroxymethyl)pyrrolidin-1- yl)methanone | 428.1 |
| 241 | 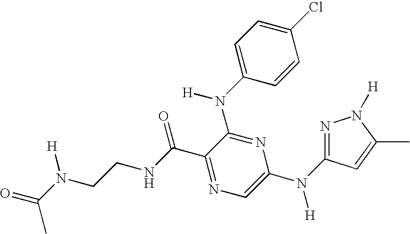 | N-(2-acetamidoethyl)-3-(4- chlorophenylamino)-5-(5-methyl- 1H-pyrazol-3-ylamino)pyrazine-2- carboxamide | 429.1 |
| 242 | 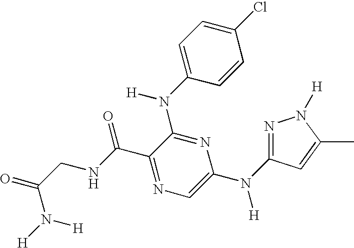 | N-(2-amino-2-oxoethyl)-3-(4- chlorophenylamino)-5-(5-methyl- 1H-pyrazol-3-ylamino)pyrazine-2- carboxamide | 401.1 |
| 243 | 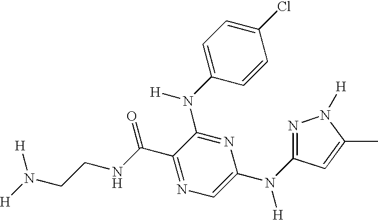 | N-(2-aminoethyl)-3-(4- chlorophenylamino)-5-(5-methyl- 1H-pyrazol-3-ylamino)pyrazine-2- carboxamide | 387.1 |
| 244 | 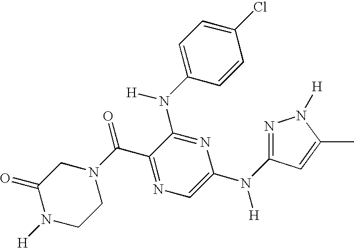 | 4-(3-(4-chlorophenylamino)-5-(5- methyl-1H-pyrazol-3- ylamino)pyrazine-2- carbonyl)piperazin-2-one | 427.0 |
| 245 | 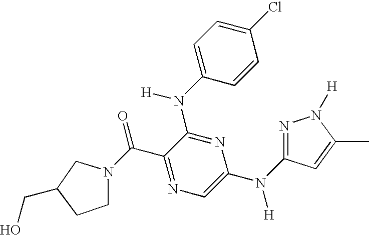 | (3-(4-chlorophenylamino)-5-(5- methyl-1H-pyrazol-3- ylamino)pyrazin-2-yl)(3- (hydroxymethyl)pyrrolidin-1- yl)methanone | 428.1 |
| 246 | 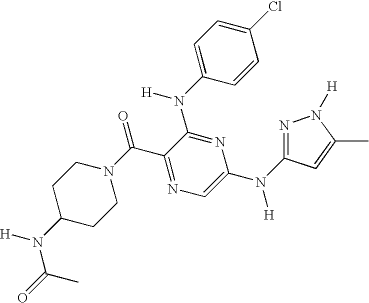 | N-(1-(3-(4-chlorophenylamino)-5-(5- methyl-1H-pyrazol-3- ylamino)pyrazine-2- carbonyl)piperidin-4-yl)acetamide | 469.5 |
| 247 | 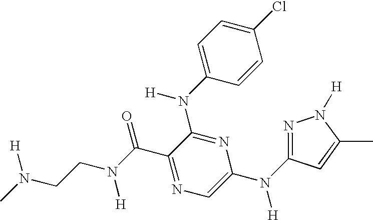 | 3-(4-chlorophenylamino)-5-(5- methyl-1H-pyrazol-3-ylamino)-N-(2- (methylamino)ethyl)pyrazine-2- carboxamide | 401.5 |
| 248 | 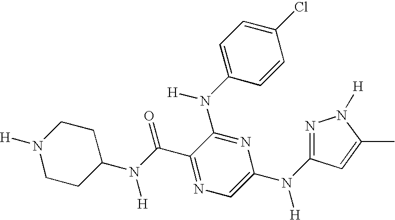 | 3-(4-chlorophenylamino)-5-(5- methyl-1H-pyrazol-3-ylamino)-N- (piperidin-4-yl)pyrazine-2- carboxamide | 427.5 |
| 249 | 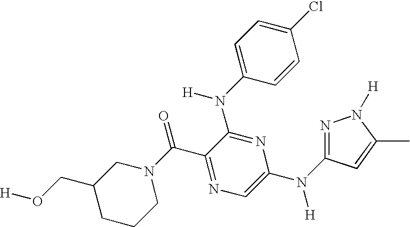 | (3-(4-chlorophenylamino)-5-(5- methyl-1H-pyrazol-3- ylamino)pyrazin-2-yl)(3- (hydroxymethyl)piperidin-1- yl)methanone | 442.5 |
| 250 | 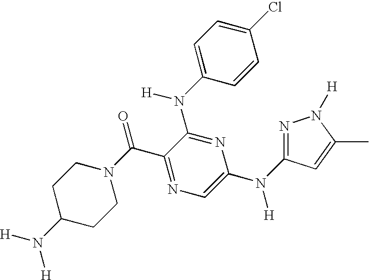 | (4-aminopiperidin-1-yl)(3-(4- chlorophenylamino)-5-(5-methyl- 1H-pyrazol-3-ylamino)pyrazin-2- yl)methanone | 427.5 |
| 251 | 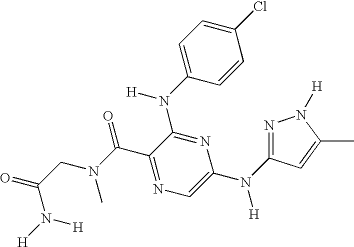 | N-(2-amino-2-oxoethyl)-3-(4- chlorophenylamino)-N-methyl-5-(5- methyl-1H-pyrazol-3- ylamino)pyrazine-2-carboxamide | 415.0 |
| 252 | 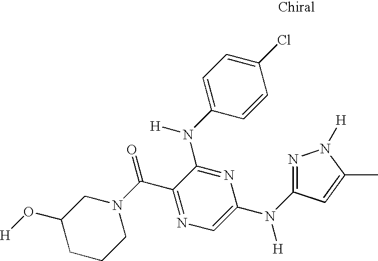 | (R)-(3-(4-chlorophenylamino)-5-(5- methyl-1H-pyrazol-3- ylamino)pyrazin-2-yl)(3- hydroxypiperidin-1-yl)methanone | 428.1 |
| 253 | 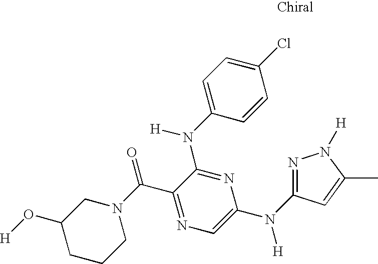 | (S)-(3-(4-chlorophenylamino)-5-(5- methyl-1H-pyrazol-3- ylamino)pyrazin-2-yl)(3- hydroxypiperidin-1-yl)methanone | 428.1 |
| 254 | 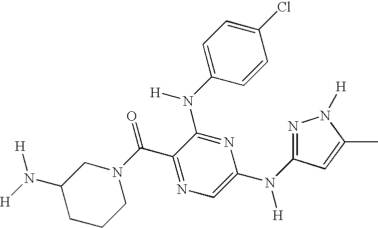 | (R)-(3-aminopiperidin-1-yl)(3-(4- chlorophenylamino)-5-(5-methyl- 1H-pyrazol-3-ylamino)pyrazin-2- yl)methanone | 427.2 |
| 255 | 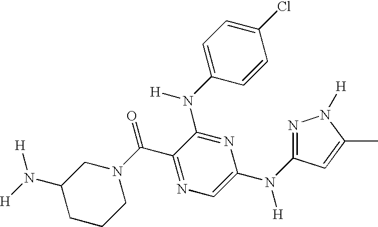 | (S)-(3-aminopiperidin-1-yl)(3-(4- chlorophenylamino)-5-(5-methyl- 1H-pyrazol-3-ylamino)pyrazin-2- yl)methanone | 427.0 |
| 256 | 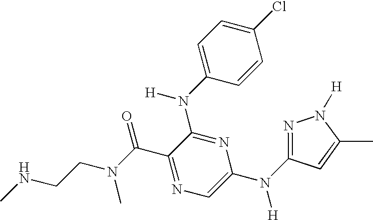 | 3-(4-chlorophenylamino)-N-methyl- 5-(5-methyl-1H-pyrazol-3-ylamino)- N-(2-(methylamino)ethyl)pyrazine- 2-carboxamide | 415.2 |
| 257 | 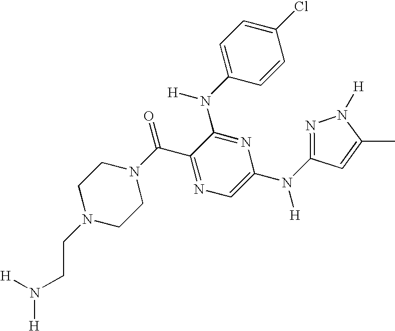 | (4-(2-aminoethyl)piperazin-1-yl)(3- (4-chlorophenylamino)-5-(5-methyl- 1H-pyrazol-3-ylamino)pyrazin-2- yl)methanone | 456.1 |
| 258 | 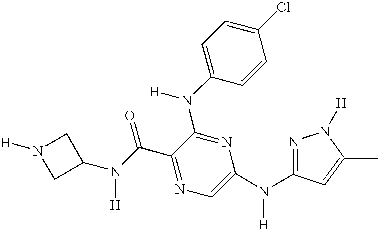 | N-(azetidin-3-yl)-3-(4- chlorophenylamino)-5-(5-methyl- 1H-pyrazol-3-ylamino)pyrazine-2- carboxamide | 399.1 |
| 259 | 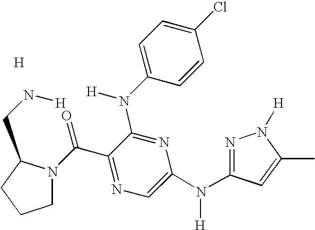 | (S)-(2-(aminomethyl)pyrrolidin-1- yl)(3-(4-chlorophenylamino)-5-(5- methyl-1H-pyrazol-3- ylamino)pyrazin-2-yl)methanone | 427.2 |
| 260 | 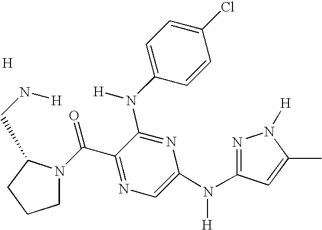 | (R)-(2-(aminomethyl)pyrrolidin-1- yl)(3-(4-chlorophenylamino)-5-(5- methyl-1H-pyrazol-3- ylamino)pyrazin-2-yl)methanone | 427.2 |
| 261 | 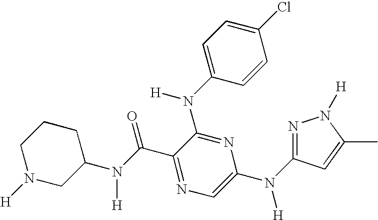 | (R)-3-(4-chlorophenylamino)-5-(5- methyl-1H-pyrazol-3-ylamino)-N- (piperidin-3-yl)pyrazine-2- carboxamide | 427.0 |
| 262 | 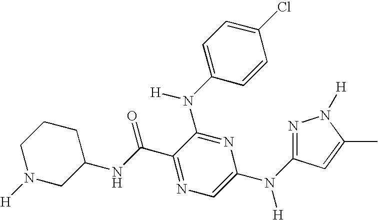 | (S)-3-(4-chlorophenylamino)-5-(5- methyl-1H-pyrazol-3-ylamino)-N- (piperidin-3-yl)pyrazine-2- carboxamide | 427.0 |
| 263 | 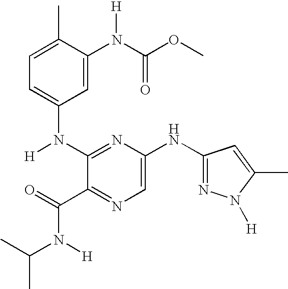 | methyl 5-(3-(isopropylcarbamoyl)-6- (5-methyl-1H-pyrazol-3- ylamino)pyrazin-2-ylamino)-2- methylphenylcarbamate | 439.4 |
| 264 | 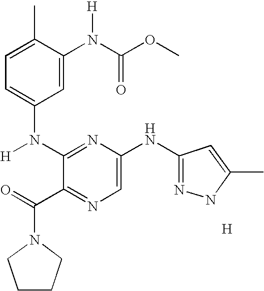 | methyl 2-methyl-5-(6-(5-methyl-1H- pyrazol-3-ylamino)-3-(pyrrolidine-1- carbonyl)pyrazin-2- ylamino)phenylcarbamate | 451.2 |

### 4.3 Methods for Preparation

The Pyrazole Pyrazine Amine Compounds can be made by one skilled in the art using conventional organic syntheses and commercially available materials. By way of example and not limitation, Pyrazole Pyrazine Amine Compounds can be prepared as outlined in Schemes 1-5 shown below, as well as in the examples set forth in Section 5.1. It should be noted that one skilled in the art can modify the procedures set forth in the illustrative schemes and examples to arrive at the desired product.

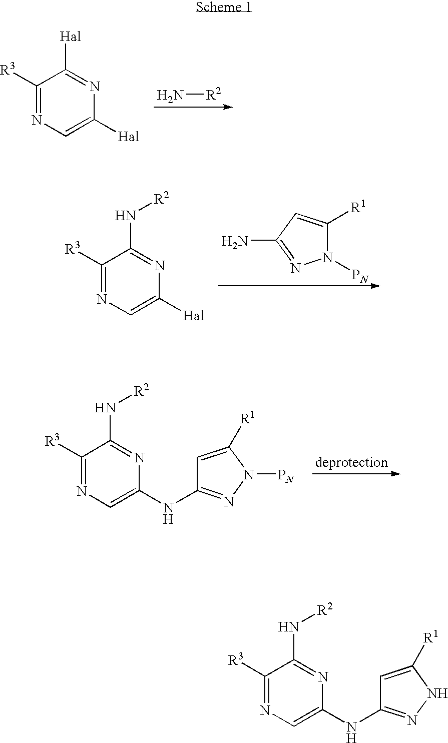

Synthesis of compounds of formula (I) wherein Q is NH, can be achieved starting with nucleophilic aromatic substitution of a dihalopyrazine with various amines R²—NH₂ (Scheme 1, wherein R¹, R² and R³ are as defined herein, Hal is a halogen, and P_{N} is an amine protecting group). This can be done under thermal conditions with the addition of a base (for example, N,N-diisopropylethylamine, triethylamine or the like) or using Buchwald conditions with a Pd catalyst and ligand, such as, for example, palladium acetate or Pd₂ dba₃ with Xantphos, in the presence of a base such as Na₂CO₃, K₂CO₃ or Cs₂CO₃. The R¹ derivatized pyrazole moiety can then be introduced under Buchwald conditions using a variety of Pd catalysts and ligands (for example, palladium acetate with Xantphos) and a base such as, for example, K₂CO₃ or Cs₂CO₃. Finally, deprotection under standard conditions affords the desired compounds of formula (I). For example, protecting groups such as Boc groups can be removed under acidic conditions (i.e. treatment with, for example, TFA or HCl), while benzylic protecting groups can be removed by hydrogenation.

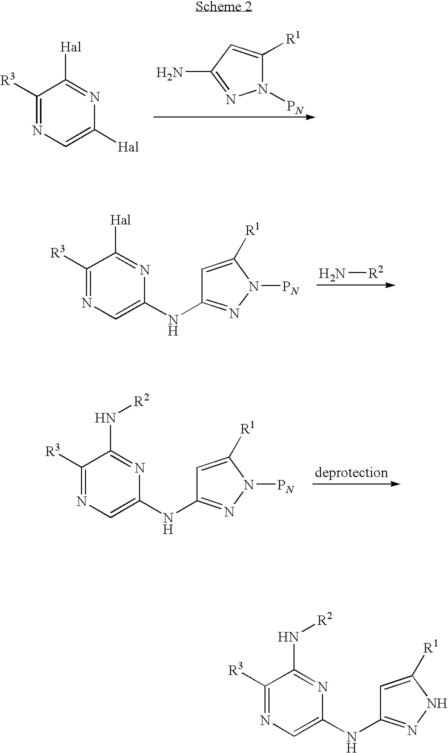

Synthesis of compounds of formula (I) wherein Q is NH, can also be obtained by installation of the R¹ derivatized pyrazole moiety first (Scheme 2, wherein R¹, R² and R³ are as defined herein, Hal is a halogen, and P_{N} is an amine protecting group). This is done under Buchwald conditions using a variety of Pd catalysts and ligands (for example, palladium acetate with Xantphos) and a base such as K₂CO₃ or Cs₂CO₃. The R² amine moiety is then introduced under Buchwald conditions with a Pd catalyst and ligand, such as, for example, palladium acetate or Pd₂ dba₃ with Xantphos, in the presence of a base such as, for example, K₂CO₃. Finally, deprotection of the amine protecting groups is achieved as before (Scheme 1).

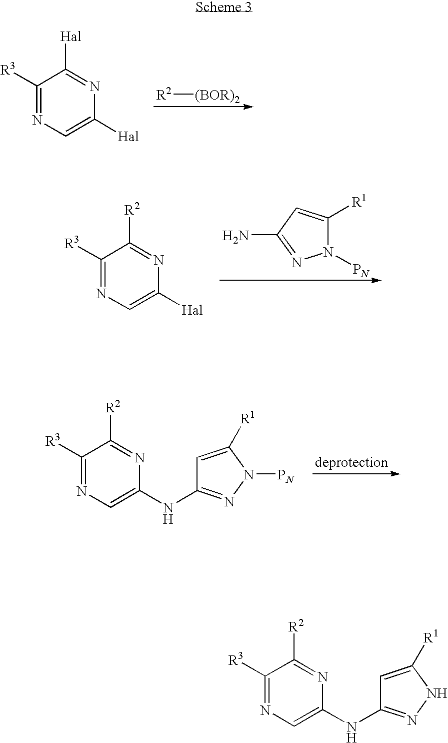

Synthesis of compounds of formula (I) wherein Q is a bond (Scheme 3, wherein R¹, R² and R³ are as defined herein, Hal is halogen and P_{N} is an amine protecting group) can be achieved starting from the coupling of a boronic acid with a dihalopyrazine, in the presence of an appropriate Pd catalyst and ligand, such as, for example, palladium acetate tetrakis(triphenylphosphine)palladium(0), in the presence of a base such as K₂CO₃. The R¹ pyrazole moiety is then installed as described for Scheme 1, followed by deprotection of the amine protecting groups, as before.

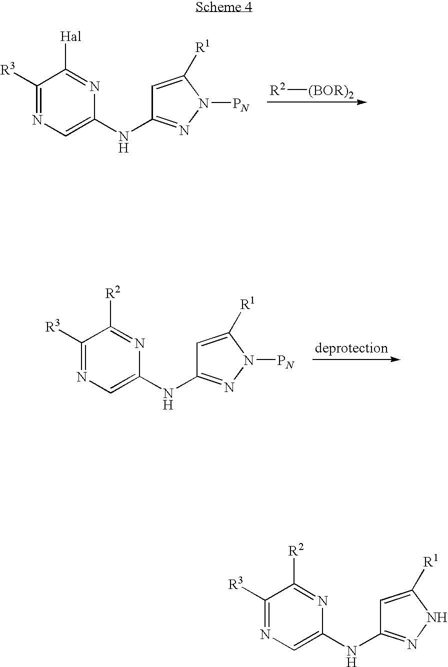

Alternatively, compounds of formula (I) wherein Q is a bond can be obtained by installation of the R¹ derivatized pyrazole moiety first (Scheme 4, wherein R¹, R² and R³ are as defined herein, Hal is halogen and P_{N} is an amine protecting group). The R² moiety is then installed by coupling of a boronic acid in the presence of an appropriate Pd catalyst and ligand, such as, for example, palladium acetate with triphenylphosphine, in the presence of a base such as K₂CO₃. Finally, deprotection of the amine protecting groups is achieved as before.

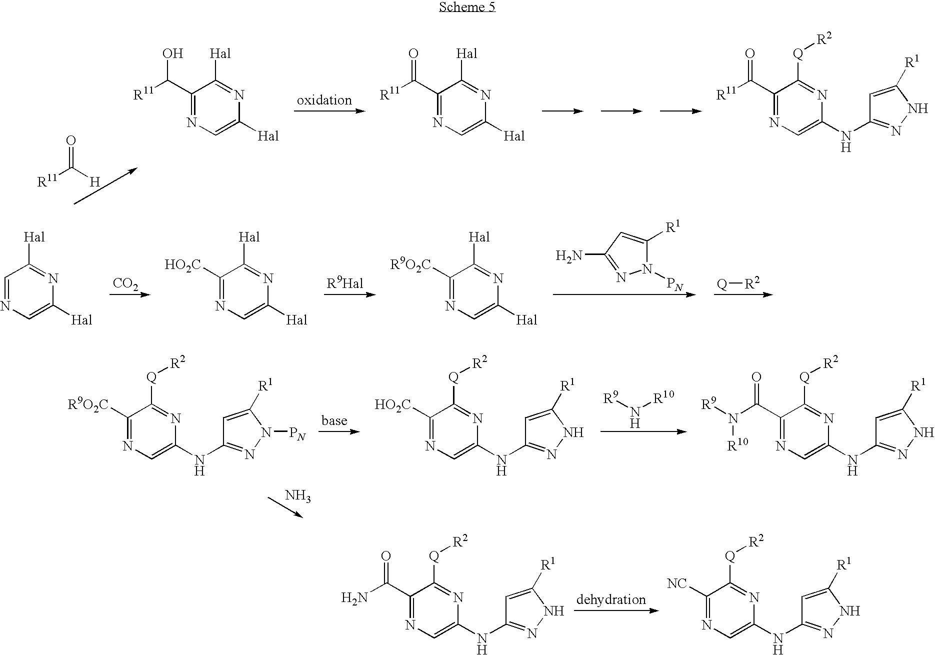

Compounds of formula (I), wherein R³ is CN, C(O)NR⁹R¹⁰, C(O)OR⁹ or C(O)R¹¹ can be obtained as shown in Scheme 5 (wherein R¹, R², R⁹, R¹⁰, R¹¹ and Q are as defined herein, and P_{N} is an amine protecting group). For compounds of formula (I), wherein R³ is C(O)R¹¹, dihalopyrazine is treated with R¹⁰C(O)H in the presence of a strong base, such as butyllithium or LTMP, followed by oxidation of the resulting alcohol to the target ketone via, for example, Dess-Martin periodinane or Jones oxidation. Installation of the R¹ derivatized pyrazole moiety and R² is achieved as described before. For compounds of formula (I), wherein R³ is C(O)NR⁹R¹⁰, R³ is introduced by treatment of dihalopyrazine with carbon dioxide in the presence of a strong base, such as, for example, LTMP, followed by esterification of the resulting carboxylic acid with R⁹—Hal (for example, with iodo methane) in the presence of a base such as K₂CO₃. Installation of the R¹ derivatized pyrazole moiety and R² is achieved as described before. Hydrolysis of the ester with a base such as hydroxide provides a carboxylic acid that can be coupled with NHR⁹R¹⁰. Alternatively, the ester can be treated with ammonia to obtain the primary amide, which is dehydrated, for example by treatment with POCl₃, to afford compounds of formula (I) wherein R³ is CN.

Coupling between amine and carboxylate-containing moieties may be effected, for example, by the use of typical amide-bond-forming reagents such as DCC, EDC, CDI, BOP, DEPBT, PyBOP, HATU, HOAt, HBTU, HCTU, TATU, TBTU, TDBTU, TSTU, and the like, or by introduction of an activating moiety on the carboxylate. The activating moiety is a sufficiently reactive leaving group to allow for amide bond formation under mild conditions. Typical activating moieties include F, Cl, Br, I, N₃, N-hydroxysuccinimide, 1-hydroxybenzotriazole, 1-hydroxy-7-azabenzotriazole, pentafluorophenol, pentachlorophenol, para-nitrophenol, or OC(O)—OR^{y}, wherein R^{y} is a C₁₋₆ alkyl group. Suitable bases include sodium bicarbonate or a suitable organoamine, such as pyridine, N-methylmorpholine, diisopropylethylamine or triethylamine. Thus, any suitable amide-bond forming procedure may be used, such as those described in Bodanszky, M. and Bodanszky, A., The Practice of Peptide Synthesis, Springer-Verlag (1984); or Jones, J. Amino Acid and Peptide Synthesis Ed. Steven G. Davies, Oxford Science (1992).

Pharmaceutically acceptable salts of the Pyrazole Pyrazine Amine Compounds can be formed by conventional and known techniques, such as by reacting a Pyrazole Pyrazine Amine Compound with a suitable acid as disclosed above. Such salts are typically formed in high yields at moderate temperatures, and often are prepared by merely isolating the compound from a suitable acidic wash in the final step of the synthesis. The salt-forming acid can be dissolved in an appropriate organic solvent, or aqueous organic solvent, such as an alkanol, ketone or ester. On the other hand, if the Pyrazole Pyrazine Amine Compound is desired in the free base form, it can be isolated from a basic final wash step, according to known techniques. For example, a typical technique for preparing hydrochloride salt is to dissolve the free base in a suitable solvent, and dry the solution thoroughly, as over molecular sieves, before bubbling hydrogen chloride gas through it.

### 4.4 Methods of Use

Pyrazole Pyrazine Amine Compounds described herein have utility as pharmaceuticals to treat or prevent disease in animals or humans. Further, Pyrazole Pyrazine Amine Compounds described herein are active against IKKs and, accordingly, are useful for the treatment and prevention of inflammatory conditions, immunological conditions, cancer, neurodegenerative diseases, age-related diseases, cardiovascular diseases, metabolic conditions, or conditions treatable or preventable by inhibition of an IKK, or an IKK pathway. Without being limited by theory, it is thought the Pyrazole Pyrazine Amine Compounds are effective for treating and preventing inflammatory conditions, immunological conditions, cancer, neurodegenerative diseases, age-related diseases, cardiovascular diseases, metabolic conditions, or conditions treatable or preventable by inhibition of an IKK, or an IKK pathway, due to their ability to modulate (e.g., inhibit) an IKK which is involved in the etiology of these conditions. Accordingly, provided herein are many uses of the Pyrazole Pyrazine Amine Compounds, including the treatment or prevention of those diseases set forth below. The methods provided herein comprise the administration of an effective amount of one or more Pyrazole Pyrazine Amine Compounds to a patient in need thereof.

In one embodiment, Pyrazole Pyrazine Amine Compounds are useful for treating or preventing inflammatory conditions, immunological conditions, cancer, neurodegenerative diseases, age-related diseases, cardiovascular diseases, metabolic conditions, or conditions treatable or preventable by inhibition of an IKK (including, but not limited to, IKK-1 and IKK-2, or an IKK pathway). In some embodiments, Pyrazole Pyrazine Amine Compounds at a concentration of 10 μM inhibit IKK2 by at least about 50%.

In a particular embodiment, provided herein are methods for the treatment or prevention of a disease or disorder associated with the inhibition of IKK-2 or the IKK-2 pathway. Particular diseases which are treatable or preventable by inhibiting IKK-2 or the IKK-2 pathway include, but are not limited to, rheumatoid arthritis; rheumatoid spondylitis; osteoarthritis; gout; asthma, bronchitis; allergic rhinitis; chronic obstructive pulmonary disease; cystic fibrosis; inflammatory bowel disease; irritable bowel syndrome; mucous colitis; ulcerative colitis; Crohn's disease; Huntington's disease; gastritis; esophagitis; hepatitis; pancreatitis; nephritis; multiple sclerosis; lupus erythematosus; Type II diabetes; obesity; atherosclerosis; restenosis following angioplasty; left ventricular hypertrophy; myocardial infarction; stroke; ischemic damages of heart, lung, gut, kidney, liver, pancreas, spleen and brain; acute or chronic organ transplant rejection; preservation of the organ for transplantation; organ failure or loss of limb (e.g., including, but not limited to, that resulting from ischemia-reperfusion injury, trauma, gross bodily injury, car accident, crush injury or transplant failure); graft versus host disease; endotoxin shock; multiple organ failure; sepsis; Guillain-Barre syndrome; psoriasis; burn from exposure to fire, chemicals or radiation; eczema; dermatitis; skin graft; ischemia; ischemic conditions associated with surgery or traumatic injury (e.g., vehicle accident, gunshot wound or limb crush); epilepsy; Alzheimer's disease; Parkinson's disease; immunological response to bacterial or viral infection; cachexia; muscle atrophy; angiogenic and proliferative diseases; solid tumor; and cancers of a variety of tissues such as colon, rectum, prostate, liver, lung, bronchus, pancreas, brain, head, neck, stomach, skin, kidney, cervix, blood, larynx, esophagus, mouth, pharynx, urinary bladder, ovary or uterine. In a specific embodiment, provided herein are methods for treating or preventing leukemia (i.e., malignant neoplasms of the blood-forming tissues) including, but not limited to, chronic lymphocytic leukemia, chronic myelocytic leukemia, acute lymphoblastic leukemia, acute myelogenous leukemia and acute myeloblastic leukemia. The leukemia can be relapsed, refractory or resistant to conventional therapy. The term “relapsed” refers to a situation where patients who have had a remission of leukemia after therapy have a return of leukemia cells in the marrow and a decrease in normal blood cells. The term “refractory or resistant” refers to a circumstance where patients, even after intensive treatment, have residual leukemia cells in their marrow.

In a particular embodiment, provide herein are methods for the treatment or prevention of a disease or disorder associated with the inhibition of IKK-2 or the IKK-2 pathway including, but not limited to, tumor syndromes resulting directly or indirectly from genetic defects in PTEN (Phosphatase and tensin homologue deleted on chromosome 10), TSC1 (Tuberous sclerosis 1), TSC2 (Tuberous sclerosis 2), NF1 (neurofibromin 1), AMPK (AMP-dependent protein kinase STK11, serine/threonine kinase 11), and LKB1. Without being limited by theory, it is thought that genetic defects associated with these proteins results in hyperactivation of the mTOR pathway. Particular diseases which are treatable or preventable through inhibition of the mTOR pathway include, but are not limited to, Cowden's disease, Cowden syndrome, Cowden-like syndrome, Bannayan-Zonana syndrome, Bannayan-Riley-Ruvalcaba syndrome, Lhermitte-Duclos disease, Endometrial carcinoma, Prostate carcinoma and Malignant melanoma, Tuberous sclerosis complex, Lymphangioleiomyomatosis, Neurofibromatosis 1, Familial hypertrophic cardiomyopathy, Peutz-jeghers syndrome, Renal Cell Carcinoma and polycystic kidney disease.

Representative inflammatory conditions that Pyrazole Pyrazine Amine Compounds are useful for treating or preventing include, but are not limited to, psoriasis, asthma, allergic rhinitis, bronchitis, chronic obstructive pulmonary disease, sepsis, reperfusion injury, cystic fibrosis, inflammatory bowel disease, irritable bowel syndrome, Crohn's disease, mucous colitis, ulcerative colitis, toxic shock syndrome, acute and chronic pain, thermal injury, adult respiratory distress syndrome (ARDS), multiple organ injury secondary to trauma, acute glomerulonephritis, dermatoses with acute inflammatory components, acute purulent meningitis, myasthenia gravis, scleroderma, atopic dermatitis, steatohepatitis, diabetes (e.g., Type I diabetes and Type II diabetes), and obesity.

Representative immunological conditions that Pyrazole Pyrazine Amine Compounds are useful for treating or preventing include, but are not limited to, rheumatoid arthritis, rheumatoid spondylitis, osteoarthritis, multiple sclerosis, lupus, inflammatory bowel disease, ulcerative colitis, Guillain-Barre Syndrome, Crohn's disease, psoriasis, graft versus host disease, myasthenia gravis, Grave's disease and diabetes (e.g., Type I and Type II diabetes).

Representative neurodegenerative diseases that Pyrimidine-2-Amine Compounds are useful for treating or preventing include, but are not limited to, Huntington's disease, Alzheimer's disease and HIV-associated encephalitis.

Representative cardiovascular diseases that Pyrazole Pyrazine Amine Compounds are useful for treating or preventing include, but are not limited to, atherosclerosis, restenosis, stroke, myocardial infarction or ischemic damage to the heart, lung, gut, kidney, liver, pancreas, spleen or brain.

Representative metabolic conditions that Pyrazole Pyrazine Amine Compounds are useful for treating or preventing include, but are not limited to, obesity and diabetes (e.g., Type I and II diabetes). In a particular embodiment, provided herein are methods for the treatment or prevention of insulin resistance. In certain embodiments, provided herein are methods for the treatment or prevention of insulin resistance that leads to diabetes (e.g., Type II diabetes). In another embodiment, provided herein are methods for the treatment or prevention of syndrome X or metabolic syndrome. In another embodiment, provide herein are methods for the treatment or prevention of diabetes. In another embodiment, provide herein are methods for the treatment or prevention of Type II diabetes, Type I diabetes, slow-onset Type I diabetes, diabetes insipidus (e.g., neurogenic diabetes insipidus, nephrogenic diabetes insipidus, dipsogenic diabetes insipidus, or gestagenic diabetes insipidus), diabetes mellitus, gestational diabetes mellitus, polycystic ovarian syndrome, maturity-onset diabetes, juvenile diabetes, insulin-dependant diabetes, non-insulin dependant diabetes, malnutrition-related diabetes, ketosis-prone diabetes, pre-diabetes (e.g., impaired glucose metabolism), cystic fibrosis related diabetes, hemochromatosis and ketosis-resistant diabetes.

In another embodiment, provided herein are methods for the treatment or prevention of fibrotic diseases and disorders. In a particular embodiment, provided herein are methods for the treatment or prevention of idiopathic pulmonary fibrosis, myelofibrosis, hepatic fibrosis, steatofibrosis and steatohepatitis, including non-alcoholic steatohepatitis (NASH).

Representative cancers that Pyrazole Pyrazine Amine Compounds are useful for treating or preventing include, but are not limited to, lymphoid-, myeloid- and epithelial-derived malignancies, including leukemia, lymphomas, myelomas, myelodysplastic syndromes, and cancers of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system. Pyrazole Pyrazine Amine Compounds are also useful for treating or preventing solid tumors and blood borne tumors. The Pyrazole Pyrazine Amine Compounds may also be useful in the treatment of cancer by enhancing the effectiveness of other chemotherapeutic agents, as described herein. Particular cancers within the scope of the methods provided herein include those associated with IKK-1 and IKK-2, or mutants or isoforms thereof

In a particular embodiment, the methods and compositions provided herein are also useful for treating, preventing or managing various types of lymphomas (i.e., a heterogenous group of neoplasms arising in the reticuloendothelial and lymphatic systems), such as Non-Hodgkin's lymphoma (NHL) (i.e., a malignant monoclonal proliferation of lymphoid cells in sites of the immune system, including lymph nodes, bone marrow, spleen, liver and gastrointestinal tract). NHLs that the Pyrazole Pyrazine Amine Compounds are useful for treating or preventing include, but are not limited to, mantle cell lymphoma, MCL, lymphocytic lymphoma of intermediate differentiation, intermediate lymphocytic lymphoma, ILL, diffuse poorly differentiated lymphocytic lymphoma, PDL, centrocytic lymphoma, diffuse small-cleaved cell lymphoma, DSCCL, follicular lymphoma, and any type of the mantle cell lymphomas that can be seen under the microscope (nodular, diffuse, blastic and mentle zone lymphoma).

The methods and compositions provided herein are also useful in the treatment or prevention of a variety of secondary disease effects, such as, but not limited to, muscle atrophy related to disease (including cancer, uremia, diabetes, and sepsis), and cancer associated bone disease (e.g. such as hypercalcemia of malignancy, osteolytic bone lesions of multiple myeloma, and osteolytic bone metastases of breast cancer, prostate cancer and other metastatic cancers). In some embodiments, the methods additionally comprise administration of a second active agent, as described herein.

Further provided herein are methods for treating patients who have been previously treated for cancer, but are non-responsive to standard therapies, as well as those who have not previously been treated. Also provided herein are methods for treating patients regardless of patient's age, although some cancers are more common in certain age groups. Still further provided herein are methods for treating patients who have undergone surgery in an attempt to treat the cancer at issue, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Particular cancers within the scope of the methods provided herein include those associated with IKK-2, Syk, Tyk2, AuroraA, cdk2, cyclinA, Ret, TrkA, Flt3, FMS, KDR or MLK, or mutants or isoforms thereof.

More particularly, cancers and related disorders that can be treated or prevented by methods and compositions provided herein include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome (or a symptom thereof such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia and chronic myelomonocytic leukemia (CMML), chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions provided herein are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, glioblastoma multiforme, neuroblastoma, glioma, and schwannomas; solid and blood born tumors; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions disclosed herein. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, kidney or uterus. In other specific embodiments, sarcoma, melanoma, or leukemia is treated or prevented.

In another embodiment, the methods and compositions provided herein are also useful for administration to patients in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiences, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy.

In another embodiment, provided herein are methods for the treatment of myeloproliferative disorders or myelodysplastic syndromes, comprising administering to a patient in need thereof an effective amount of a Pyrazole Pyrazine Amine Compound or a composition thereof. In certain embodiments, the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

In another embodiment, provided herein are methods for the treatment of cancer or tumors resistant to other kinase inhibitors such as imatinib mesylate (STI-571 or Gleevec™) treatment, comprising administering to a patient in need thereof an effective amount of a Pyrazole Pyrazine Amine Compound or a composition thereof. In a particular embodiment, provided herein are methods for the treatment of leukemias, including, but not limited to, gastrointestinal stromal tumor (GIST), acute lymphocytic leukemia or chronic myelocytic leukemia resistant to imatinib mesylate (STI-571 or Gleevec™) treatment, comprising administering to a patient in need thereof an effective amount of a Pyrazole Pyrazine Amine Compound or a composition thereof.

The various types of the cancers are described in U.S. provisional application No. 60/380,842, filed May 17, 2002, the entireties of which are incorporated herein by reference (see, e.g., Section 2.2. Types of Cancers). Specific cancers include, but are not limited to, leukemias such as chronic lymphocytic leukemia, chronic myelocytic leukemia, acute lymphoblastic leukemia, acute myelogenous leukemia, and acute myeloblastic leukemia; advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scieroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In one embodiment, the cancer is primary or metastatic. In another embodiment, the cancer is relapsed, refractory or resistance to chemotherapy or radiation; in particular, refractory to thalidomide.

Further provide herein are methods for treating patients who have been previously treated for cancer, but are non-responsive to standard therapies, as well as those who have not previously been treated. Also provided herein are methods for treating patients regardless of patient's age, although some cancers are more common in certain age groups. Still further provided herein are methods for treating patients who have undergone surgery in an attempt to treat the cancer at issue, as well as those who have not. Because patients with cancer have heterogenous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

A Pyrazole Pyrazine Amine Compound can be combined with other pharmacologically active compounds (“second active agents” hereafter also referred to as ingredient(s) A) in methods and compositions described herein. It is believed that certain combinations may work synergistically in the treatment of particular types diseases or disorders, and conditions and symptoms associated with such diseases or disorders. A Pyrazole Pyrazine Amine Compound can also work to alleviate adverse effects associated with certain second active agents, and vice versa.

One or more second active ingredients or agents can be used in the methods and compositions described herein. Second active agents can be large molecules (e.g., proteins) or small molecules (e.g., synthetic inorganic, organometallic, or organic molecules). In a particular embodiment, the second active agent is an inhibitor of IKK-2 or the IKK-2 pathway.

Examples of large molecule second active agents include, but are not limited to, hematopoietic growth factors, cytokines, and monoclonal and polyclonal antibodies. Specific examples of large molecules include etanercept, infliximab, alefacept, adalimumab, efalizumab, anakinra, IL-1RA, alpha-interferon, interferon beta 1β, CTLA 4, and other antibodies or receptor constructs directed against TNFα, IL-1 and IL 6, LFA-1, or C5.

Additional ingredient(s) A can be anti-CD40 monoclonal antibodies (such as, for example, SGN-40); histone deacetylyase inhibitors (such as, for example, SAHA and LAQ 824); heat-shock protein-90 inhibitors (such as, for example, 17-AAG); insulin-like growth factor-1 receptor kinase inhibitors; vascular endothelial growth factor receptor kinase inhibitors (such as, for example, PTK787); insulin growth factor receptor inhibitors; lysophosphatidic acid acyltransrerase inhibitors; IkB kinase inhibitors; p38MAPK inhibitors; EGFR inhibitors (such as, for example, gefitinib and erlotinib HCL); HER-2 antibodies (such as, for example, trastuzumab (Herceptin® and pertuzumab (Omnitarg™); VEGFR antibodies (such as, for example, bevacizumab (Avastin™); VEGFR inhibitors (such as, for example, flk-1 specific kinase inhibitors, SU5416 (semaxanib) and ptk787/zk222584); P13K inhibitors (such as, for example, wortmannin); C-Met inhibitors (such as, for example, PHA-665752); an ImiDs® brand Immunomodulatory product (for example thalidomide, lenalidomide or pomalidomide), monoclonal antibodies (such as, for example, rituximab (Rituxan®), tositumomab (Bexxar®), edrecolomab (Panorex®) and G250); and anti-TNF-α antibodies. Examples of small molecule active agents include, but are not limited to, small molecule anti-cancer agents and antibiotics (e.g., clarithromycin, doxycycline).

Specific second active compounds that can be combined with a Pyrazole Pyrazine Amine Compound vary depending on the specific indication to be treated, prevented or managed. For example, non-steroidal anti-inflammatory drugs (NSAIDs), which are widely used for the treatment of inflammation, pain and fever, may be used. Such NSAIDs include acetaminophen, aspirin, ibuprofen, choline magnesium salicylate, choline salicylate, diclofenac, diflunisal, etodolac, fenoprofen calcium, flurbiprofen, indomethacin, ketoprofen, carprofen, indoprofen, ketorolac tromethamine, magnesium salicylate, meclofenamate sodium, mefenamic acid, oxaprozin, piroxicam, sodium salicylate, sulindac, tolmetin, meloxicam, rofecoxib, celecoxib, etoricoxib, valdecoxib, nabumetone, naproxen, lomoxicam, nimesulide, indoprofen, remifenzone, saisalate, tiaprofenic acid, flosulide, and the like, or a combination of two or more thereof.

Angiogenesis inhibitors may serve as ingredient(s) A, such as VEGF inhibitors, taxol, pentoxyfylline and/or thalidomide. In one embodiment, the ingredient(s) A is any SelCid™ or ImiDs® brand Immunomodulatory products. In a particular embodiment, the ingredient(s) A is thalidomide, lenalidomide, pomalidomide, or a combination of two or more thereof. In one embodiment, the ingredient(s) A is Velcade or Vidaza.

Also contemplated as ingredient(s) A are steroids, such as glucocorticoids, and vitamin D3 and analogs thereof (cholecalciferols), alone (the latter being used mostly for psoriasis) or in combination. Steroids include budesonide, dexamethasone, fluocinonide, hydrocortisone, betamethasone, halobetasol (ulobetasol), methylprednisolone, prednisolone, prednisone, clobetasone, deflazacort, fhiocinolone acetonide, fluticasone, triamcinolone acetonide, mometasone and diflucortolone. Among vitamin D3 derivatives are calcipotriol, tacalcitol, maxacalcitol, and tacalitol, the calciotropic hormones, 1α,2,5-dihydroxyvitamin D3, and parathyroid hormone-related peptide.

Many types of immunomodulatory, immunosuppressive or cytostatic drugs can be used in combination with compounds as described herein. Exemplary agents include hydroxychloroquine, D-penicillamine, sulfasalazine, auranofin, gold sodium thiomalate, minocycline, dapsone, chlorambucil, mercaptopurine, tacrolimus, sirolimus, pimecrolimus, mycophenolate mofetil, cyclosporine, leflunomide, methotrexate, azathioprine, cyclophosphamide, macrolides, ascomycin, hydroxyurea, 6-thioguanine, (Orfanos C E., Cutis 64(5), 347-353 (1999)); alefacept, leflunomide, infliximab, etanercept, efalizumab, anti-CD4, anti-CD25, peptide T, LFA3TIP, alicaforsen, DAB389, CTLA 4Ig, anti-CD80, for example IDEC-114 or ABX-IL8, DAB-IL-2, IL-10, anti-TAC, basiliximab and daclizumab. In addition, agents or therapies which act on other targets or immune mediated products are suitable as the ingredient(s) A. These include, for example, inhibitors of protein tyrosine kinases (PTKs) such as epidermal growth factor receptor (EGFR), E-selectin inhibitors, and therapies widely used for psoriasis such as anthralin, coal tar, phototherapies including ultraviolet B (UVB) or psoralens ultraviolet A (PUVA), photodynamic therapy and laser therapy.

Retinoid therapy can also be used as ingredient(s) A. Thus, for example, bexarotene, acitretin, etretinate, tazarotene, hydroxyurea, 6-thioguanine and phototherapies are suitable additional ingredients. (Orfanos C E., Cutis 64(5), 347-353 (1999); see also Saurat J H., J. Am. Acad. Derm. 41(3 Pt 2), S2-S6 (1999)).

Ingredients A useful in the methods as described herein further include small molecule inhibitors directed against enzymes involved in signal transduction pathways or to cell adhesion molecules like LFA-1 or ICAM-1.

In some embodiments, the method of treating cancer (as described herein) further comprises treating the subject with surgery, radiation, cryotherapy, or one or more antiproliferative agents or a combination thereof. In some such embodiments, the antiproliferative agent is an alkylating agent, platinum agent, antimetabolite, topoisomerase inhibitor, antitumor antibiotic, antimitotic agent, aromatase inhibitor, thymidylate synthase inhibitor, DNA antagonist, farnesyltransferase inhibitor, pump inhibitor, histone acetyltransferase inhibitor, metalloproteinase inhibitor, ribonucleoside reductase inhibitor, endothelin A receptor antagonist, retinoic acid receptor agonist, immunomodulator, hormonal or antihormonal agent, photodynamic agent, angiogenesis inhibitor, apoptosis inducer, or a tyrosine kinase inhibitor. In some of these embodiments, the alkylating agent is busulfan, procarbazine, ifosfamide, altretamine, hexamethylmelamine, estramustine phosphate, thiotepa, mechlorethamine, dacarbazine, streptozocin, lomustine, temozolomide, cyclophosphamide, semustine, or chlorambucil. Examples of platinum agents include spiroplatin, lobaplatin (Aeterna), tetraplatin, satraplatin (Johnson Matthey), ormaplatin, iproplatin, miriplatin (Sumitomo), nexplatin (AnorMED), polymer platinate (Access), oxaliplatin, or carboplatin. In some embodiments, the antimetabolite is azacytidine, trimetrexate, floxuridine, deoxycoformycin, 2-chlorodeoxyadenosine, pentostatin, 6-mercaptopurine, hydroxyurea, 6-thioguanine, decitabine (SuperGen), cytarabine, clofarabine (Bioenvision), 2-fluorodeoxy cytidine, irofulven (MGI Pharma), methotrexate, tomudex, ethynylcytidine (Taiho), fludarabine, gemcitabine, raltitrexed, or capecitabine. In others, the topoisomerase inhibitor is amsacrine, exatecan mesylate (Daiichi), epirubicin, quinamed (ChemGenex), etoposide, gimatecan (Sigma-Tau), teniposide, mitoxantrone, diflomotecan (Beaufour-Ipsen), 7-ethyl-10-hydroxy-camptothecin, dexrazoxanet (TopoTarget), elsamitrucin (Spectrum), pixantrone (Novusphamma), edotecarin (Merck & Co), becatecarin (Exelixis), karenitecin (BioNumerik), BBR-3576 (Novuspharma), belotecan (Chong Kun Dang), rubitecan (SuperGen), irinotecan (CPT-11), or topotecan. In yet others, the antitumor antibiotic is dactinomycin (actinomycin D), doxycycline, azonafide, valrubicin, anthrapyrazole, daunorubicin (daunomycin), oxantrazole, therarubicin, losoxantrone, idarubicin, bleomycinic acid, rubidazone, sabarubicin (Menarini), plicamycinp, 13-deoxydoxorubicin hydrochloride (Gem Pharmaceuticals), porfiromycin, epirubicin, mitoxantrone (novantrone) or amonafide. Examples of antimitotic agents are colchicines, ABT-751 (Abbott), vinblastine, xyotax (Cell Therapeutics), vindesine, IDN 5109 (Bayer), dolastatin 10 (NCl), A 105972 (Abbott), rhizoxin (Fujisawa), A 204197 (Abbott), mivobulin (Warner-Lambert), synthadotin (BASF), cemadotin (BASF), indibulin (ASTAMedica), RPR 109881A (Aventis), TXD 258 (Aventis), combretastatin A4 (BMS), epothilone B (Novartis), isohomohalichondrin-B (PharmaMar), T 900607 (Tularik), ZD 6126 (AstraZeneca), batabulin(Tularik), cryptophycin 52 (Eli Lilly), vinflunine (Fabre), hydravin (Prescient NeuroPharma), auristatin PE (Teikoku Hormone), azaepothilone B (BMS), ixabepilone (BMS), tavocept (BioNumerik), BMS 184476 (BMS), combrestatin A4 disodium phosphate (OXiGENE), BMS 188797 (BMS), dolastatin-10 (NIH), taxoprexin (Protarga), cantuzumab mertansine (GlaxoSmithKline), docetaxel, vinorelbine, or vincristine. In some embodiments, the aromatase inhibitor is aminoglutethimide, atamestane (BioMedicines), formestane, fadrozole, letrozole, exemestane, or anastrazole. In others, the thymidylate synthase inhibitor is pemetrexed (Eli Lilly), nolatrexed (Eximias), ZD-9331 (BTG), doxifluridine (Nippon Roche), or 5,10-methylenetetrahydrofolate (BioKeys). In yet others, the DNA antagonist is trabectedin (PharmaMar), edotreotide (Novartis), glufosfamide (Baxter International), mafosfamide (Baxter International), apaziquone (Spectrum Pharmaceuticals), or thymectacin (NewBiotics). In still others, the farnesyltransferase inhibitor is arglabin (NuOncology Labs), tipifarnib (Johnson & Johnson), lonafarnib (Schering-Plough), perillyl alcohol (DOR BioPharma), or sorafenib (Bayer). Examples of pump inhibitors are zosuquidar trihydrochloride (Eli Lilly), tariquidar (Xenova), biricodar dicitrate (Vertex), or MS-209 (Schering AG). Examples of histone acetyltransferase inhibitors include tacedinaline (Pfizer), pivaloyloxymethyl butyrate (Titan), AP-CANC-03 and AP-CANC-04 (Aton Pharma), depsipeptide (Fujisawa), or MS-275 (Schering AG). In some embodiments, the metalloproteinase inhibitor is neovastat (Aeterna Laboratories), metastat (CollaGenex), or marimastat (British Biotech). In others, the ribonucleoside reductase inhibitor is gallium maltolate (Titan), tezacitabine (Aventis), triapine (Vion), or didox (Molecules for Health). In yet others, the endothelin A receptor antagonist is atrasentan (Abbott), bosentan (Roche), ambrisentan (BASF), sitaxsentan (Encysive), clazosentan (Roche), darusentan (Knoll), and ZD-4054 (AstraZeneca). In still others, the retinoic acid receptor agonist is fenretinide (Johnson & Johnson), alitretinoin (Ligand), tazarotene (Allergan), tetrinoin (Roche), isotretinoin (Roche), 13-cis-retinoic acid (UCSD), or LGD-1550 (Ligand). In some embodiments, the immuno-modulator is interferon, Roferon-A (Roche), infliximab (Centocor), dexosome therapy (Anosys), oncophage (Antigenics), pentrix (Australian Cancer Technology), GMK vaccine (Progenies), CD 154 cell therapy (Tragen), adenocarcinoma vaccine (Biomira), transvax (Intercell), avicine (AVI BioPharma), norelin (Biostar), IRX-2 (Immuno-Rx), BLP-25 liposome vaccine (Biomira), PEP-005 (Peplin Biotech), multiganglioside vaccine (Progenies), synchrovax vaccine (CTL Immuno), b-alethine (Dovetail), melanoma vaccine (CTL Immuno), vasocare (Vasogen), rituximab (Genentech/Biogen Idee), or p21 RAS vaccine (GemVax). In others, the hormonal agent is an estrogen, dexamethasone, a conjugated estrogen, prednisone, ethinyl estradiol, methylprednisolone, chlortrianisen, prednisolone, idenestrol, aminoglutethimide, hydroxyprogesterone caproate, leuprolide, medroxyprogesterone, octreotide, testosterone, mitotane, testosterone propionate, fluoxymesterone, methyltestosterone, 2-methoxyestradiol (EntreMed), diethylstilbestrol, arzóxifene (Eli Lilly), megestrol, tamoxifen, bicalutamide, toremofine, fiutamide, goserelin, nilutamide, or leuporelin. In yet others, the photodynamic agent is talaporfin (Light Sciences), Pd-bacteriopheophorbide (Yeda), theralux (Theratechnologies), lutetium texaphyrin (Pharmacyclics), motexafin, gadolinium (Pharrhacyclics), or hypericin. In still others, the angiogenesis inhibitor is neovastat (Aetema Zentaris), ATN-224 (Attenuon), sorafenib (Bayer), thalidomide, pomalidomide, lenalidomide, bevacizumab (Genentech), ranibizumab (Genentech), benefin (Lane Labs), L-651582 (Merck & Co), vatalanib (Novartis), or sutent (Sugen). In some embodiments, the apoptosis inducer is TRAIL (tumor necrosis factor-related apoptosis inducing ligand) or bortezomib. Examples of tyrosine kinase inhibitors include imatinib (Novartis), leflunomide (Sugen/Pharmacia), kahalide F (PharmaMar) iressa (AstraZeneca), lestaurtinib (Cephalon), erlotinib (Oncogene Science), canertinib (Pfizer), tandutinib (Millenium), squalamine (Genaera), midostaurin (Novartis), phenoxodiol, SU6668 (Pharmacia), cetuximab (ImClone), rhu-Mab (Genentech), ZD6474 (AstraZeneca), MDX-H210 (Medarex), vatalanib (Novartis), omnitarg (Genentech), lapatinib (GlaxoSmithKline), panitumumab (Abgenix), IMC-Icl 1 (ImClone), sorafenib (Bayer) or trastuzumab (Genentech). In some embodiments, the antiproliferative agent is melphalan, carmustine, cisplatin, 5-fluorouracil, mitomycin C, adriamycin (doxorubicin), bleomycin, or paclitaxel (Taxol®). In one embodiment, the antiproliferative agent is any SelCid™ or ImiDs® brand Immunomodulatory product, in particular thalidomide, lenalidomide, pomalidomide, Velcade, Vidaza, or a combination of two or more thereof.

In some embodiments, the methods of treating cancers (as described herein) further comprise treatment with active agents useful in the treatment of secondary disease effects, for example, bone disease. Examples of such agents include bisphosphonates.

For the treatment, prevention or management of cancer, second active agents include, but are not limited to: semaxanib; cyclosporin; etanercept; doxycycline; bortezomib; acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other second agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clathromycin; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (Gleevece), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterial cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, 2-methoxyestradiol, telomestatin, inducers of apoptosis in multiple myeloma cells (such as, for example, TRAIL), bortezomib, statins, semaxanib, cyclosporin, etanercept, doxycycline, bortezomib, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

Similarly, examples of specific second agents according to the indications to be treated, prevented, or managed can be found in the following references, all of which are incorporated herein in their entireties: U.S. Pat. Nos. 6,281,230 and 5,635,517; U.S. application Ser. Nos. 10/411,649, 10/483,213, 10/411,656, 10/693,794, 10/699,154, and 10/981,189; and U.S. provisional application Nos. 60/554,923, 60/565,172, 60/626,975, 60/630,599, 60/631,870, and 60/533,862.

Examples of additional second active agents include, but are not limited to, conventional therapeutics used to treat or prevent pain such as antidepressants, anticonvulsants, antihypertensives, anxiolytics, calcium channel blockers, muscle relaxants, non-narcotic analgesics, opioid analgesics, anti-inflammatories, cox-2 inhibitors, immunomodulatory agents, alpha-adrenergic receptor agonists or antagonists, immunosuppressive agents, corticosteroids, byperbaric oxygen, ketamine, other anesthetic agents, NMDA antagonists, and other therapeutics found, for example, in the Physician's Desk Reference 2003. Specific examples include, but are not limited to, salicylic acid acetate (Aspirin®), celecoxib (Celebrex®), Enbrel®, ketamine, gabapentin (Neurontin®), phenyloin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofianil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®), mexiletine, nifedipine, propranolol, tramadol, lamotrigine, ziconotide, ketamine, dextromethorphan, benzodiazepines, baclofen, tizanidine and phenoxybenzamine.

Further examples of additional second active agents include, but are not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound or an antiangiogenesis compound, or a combination thereof. Specific examples include, but are not limited to, verteporfin, purlytin, an angiostatic steroid, rhuFab, interferon-2ÿ, pentoxifylline, tin etiopurpurin, motexafin lutetium, 9-fluoro-11,21-dihydroxy-16, 17-1-methylethylidinebis(oxy)pregna-1,4-diene-3,20-dione, latanoprost (see U.S. Pat. No. 6,225,348), tetracycline and its derivatives, rifamycin and its derivatives, macrolides, metronidazole (U.S. Pat. Nos. 6,218,369 and 6,015,803), genistein, genistin, 6′-O-Mal genistin, 6′-O-Ac genistin, daidzein, daidzin, 6′-O-Mal daidzin, 6′-O-Ac daidzin, glycitein, glycitin, 6′-O-Mal glycitin, biochanin A, formononetin (U.S. Pat. No. 6,001,368), triamcinolone acetomide, dexarnethasone (U.S. Pat. No. 5,770,589), thalidomide, glutathione (U.S. Pat. No. 5,632,984), basic fibroblast growth factor (bFGF), transforming growth factor b (TGF-b), brain-derived neurotrophic factor (BDNF), plasminogen activator factor type 2 (PAI-2), EYE101 (Eyetech Pharmaceuticals), LY333531 (Eli Lilly), Miravant, and RETISERT implant (Bausch & Lomb). All of the references cited above are incorporated herein in their entireties by reference.

Further examples of additional second active agents include, but are not limited to, keratolytics, retinoids, α-hydroxy acids, antibiotics, collagen, botulinum toxin, interferon, and immunomodulatory agents. Specific examples include, but are not limited to, 5-fluorouracil, masoprocol, trichloroacetic acid, salicylic acid, lactic acid, ammonium lactate, urea, tretinoin, isotretinoin, antibiotics, collagen, botulinum toxin, interferon, corticosteroid, transretinoic acid and collagens such as human placental collagen, animal placental collagen, Dermalogen, AlloDerm, Fascia, Cymetra, Autologen, Zyderm, Zyplast, Resoplast, and Isolagen.

Further examples of additional second active agents include, but are not limited to, anticoagulants, diuretics, cardiac glycosides, calcium channel blockers, vasodilators, prostacyclin analogues, endothelin antagonists, phosphodiesterase inhibitors (e.g., PDE V inhibitors), endopeptidase inhibitors, lipid lowering agents, thromboxane inhibitors, and other therapeutics known to reduce pulmonary artery pressure. Specific examples include, but are not limited to, warfarin (Coumadin®), a diuretic, a cardiac glycoside, digoxin-oxygen, diltiazem, nifedipine, a vasodilator such as prostacyclin (e.g., prostaglandin I2 (PGI2), epoprostenol (EPO, Floran®), treprostinil (Remodulin®), nitric oxide (NO), bosentan (Tracleer®), amlodipine, epoprostenol (Floran®), treprostinil (Remodulin®), prostacyclin, tadalafil (Clalis®), simvastatin (Zocor®), omapatrilat (Vanlev®), irbesartan (Avapro®), pravastatin (Pravachol®), digoxin, L-arginine, iloprost, betaprost, and sildenafil (Viagra®).

Further examples of additional second active agents include, but are not limited to, anthracycline, platinum, alkylating agent, oblimersen (Genasense®), cisplatinum, cyclophosphamide, temodar, carboplatin, procarbazine, gliadel, tamoxifen, topotecan, methotrexate, taxotere, irinotecan, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, bleomycin, hyaluronidase, mitomycin C, mepacrine, thiotepa, tetracycline and gemcitabine.

Further examples of additional second active agents include, but are not limited to, chloroquine, quinine, quinidine, pyrimethamine, sulfadiazine, doxycycline, clindamycin, mefloquine, halofantrine, primaquine, hydroxychloroquine, proguanil, atovaquone, azithromycin, suramin, pentamidine, melarsoprol, nifurtimox, benznidazole, amphotericin B, pentavalent antimony compounds (e.g., sodium stiboglucuronate), interfereon gamma, itraconazole, a combination of dead promastigotes and BCG, leucovorin, corticosteroids, sulfonamide, spiramycin, IgG (serology), trimethoprim, and sulfamethoxazole.

Further examples of additional second active agents include, but are not limited to: antibiotics (therapeutic or prophylactic) such as, but not limited to, ampicillin, clarithromycin, tetracycline, penicillin, cephalosporins, streptomycin, kanamycin, and erythromycin; antivirals such as, but not limited to, amantadine, rimantadine, acyclovir, and ribavirin; immunoglobulin; plasma; immunologic enhancing drugs such as, but not limited to, levamisole and isoprinosine; biologics such as, but not limited to, gammaglobulin, transfer factor, interleukins, and interferons; hormones such as, but not limited to, thymic; and other immunologic agents such as, but not limited to, B cell stimulators (e.g., BAFF/BlyS), cytokines (e.g., IL-2, IL-4, and IL-5), growth factors (e.g., TGF-\ddot{γ}), antibodies (e.g., anti-CD40 and IgM), oligonucleotides containing unmethylated CpG motifs, and vaccines (e.g., viral and tumor peptide vaccines).

Further examples of additional second active agents include, but are not limited to: a dopamine agonist or antagonist, such as, but not limited to, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, and Symmetrel; a MAO inhibitor, such as, but not limited to, iproniazid, clorgyline, phenelzine and isocarboxazid; a COMT inhibitor, such as, but not limited to, tolcapone and entacapone; a cholinesterase inhibitor, such as, but not limited to, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, and demecarium; an anti-inflammatory agent, such as, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids; and an antiemetic agent, such as, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinol, thiethylperazine, thioproperazine, tropisetron, and a mixture thereof.

Further examples of additional second active agents include, but are not limited to, immunomodulatory agents, immunosuppressive agents, antihypertensives, anticonvulsants, fibrinolytic agents, antiplatelet agents, antipsychotics, antidepressants, benzodiazepines, buspirone, amantadine, and other known or conventional agents used in patients with CNS injury/damage and related syndromes. Specific examples include, but are not limited to: steroids (e.g., glucocorticoids, such as, but not limited to, methylprednisolone, dexamethasone and betamethasone); an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone; a cAMP analog including, but not limited to, db-cAMP; an agent comprising a methylphenidate drug, which comprises 1-threo-methylphenidate, d-threo-methylphenidate, dl-threo-methylphenidate, 1-erythro-methylphenidate, d-erythro-methylphenidate, dl-erythro-methylphenidate, and a mixture thereof; and a diuretic agent such as, but not limited to, mannitol, furosemide, glycerol, and urea.

Further examples of additional second active agents include, but are not limited to, a tricyclic antidepressant agent, a selective serotonin reuptake inhibitor, an antiepileptic agent (gabapentin, pregabalin, carbamazepine, oxcarbazepine, levitiracetam, topiramate), an antiaryhthmic agent, a sodium channel blocking agent, a selective inflammatory mediator inhibitor, an opioid agent, a second immunomodulatory compound, a combination agent, and other known or conventional agents used in sleep therapy. Specific examples include, but are not limited to, Neurontin, oxycontin, morphine, topiramate, amitryptiline, nortryptiline, carbamazepine, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, Symmetrel, iproniazid, clorgyline, phenelzine, isocarboxazid, tolcapone, entacapone, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, demecarium, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone, benzbromarone, betamethasone and other glucocorticoids, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and a mixture thereof.

Further examples of additional second active agents include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; and G-CSF; hydroxyurea; butyrates or butyrate derivatives; nitrous oxide; HEMOXIN™ NIPRISAN™; see U.S. Pat. No. 5,800,819); Gardos channel antagonists such as clotrimazole and triaryl methane derivatives; Deferoxamine; protein C; and transfusions of blood, or of a blood substitute such as Hemospan™ or Hemospan™ PS (Sangart).

In some embodiments, a Pyrazole Pyrazine Amine Compound is administered as adjuvant therapy to standard cancer therapy. Standard cancer therapies include surgery, radiation therapy, bone marrow transplantation, chemotherapeutic treatment, biological response modifier treatment, and certain combinations of the foregoing, as described herein.

Administration of a Pyrazole Pyrazine Amine Compound and a second active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for Pyrazole Pyrazine Amine Compounds is oral. Preferred routes of administration for the second active agents or ingredients of the invention are known to those of ordinary skill in the art. See, e.g., Physicians' Desk Reference, 1755-1760 (56^{th} ed., 2002).

In one embodiment, the second active agent is administered intravenously or subcutaneously. In another embodiment, the second active agent is administered intravenously or subcutaneously once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of a Pyrazole Pyrazine Amine Compound and any optional additional active agents concurrently administered to the patient.

Further provided herein are methods of reducing, treating and/or preventing adverse or undesired effects associated with conventional therapy including, but not limited to, surgery, chemotherapy, radiation therapy, hormonal therapy, biological therapy and immunotherapy. Pyrazole Pyrazine Amine Compounds and other active ingredients can be administered to a patient prior to, during, or after the occurrence of the adverse effect associated with conventional therapy.

### 4.5 Pharmaceutical Compositions and Routes of Administration

The Pyrazole Pyrazine Amine Compounds can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the Pyrazole Pyrazine Amine Compound in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of a Pyrazole Pyrazine Amine Compound to be administered to a patient is rather widely variable and can be subject to the judgment of a health-care practitioner. In general, the Pyrazole Pyrazine Amine Compounds can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the Pyrazole Pyrazine Amine Compound administered will depend on such factors as the solubility of the active component, the formulation used and the route of administration.

In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Pyrazole Pyrazine Amine Compound to a patient in need thereof.

In another embodiment, provided herein are methods for the treatment or prevention of a disease or disorder comprising the administration of about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of a Pyrazole Pyrazine Amine Compound to a patient in need thereof. In a particular embodiment, the methods disclosed herein comprise the administration of 400 mg/day, 600 mg/day or 800 mg/day of a Pyrazole Pyrazine Amine Compound to a patient in need thereof.

In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of a Pyrazole Pyrazine Amine Compound.

In a particular embodiment, provided herein are unit dosage formulation comprising about 100 mg or 400 mg of a Pyrazole Pyrazine Amine Compound.

In another embodiment, provided herein are unit dosage formulations that comprise 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a Pyrazole Pyrazine Amine Compound.

A Pyrazole Pyrazine Amine Compound can be administered once, twice, three, four or more times daily. In a particular embodiment, doses of 600 mg or less are administered as a once daily dose and doses of more than 600 mg are administered twice daily in an amount equal to one half of the total daily dose.

A Pyrazole Pyrazine Amine Compound can be administered orally for reasons of convenience. In one embodiment, when administered orally, a Pyrazole Pyrazine Amine Compound can be administered with a meal and water. In another embodiment, the Pyrazole Pyrazine Amine Compound is dispersed in water, milk or juice (e.g., apple juice or orange juice) and administered orally as a suspension.

The Pyrazole Pyrazine Amine Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a Pyrazole Pyrazine Amine Compound without an additional carrier, excipient or vehicle.

In another embodiment, provided herein are compositions comprising an effective amount of a Pyrazole Pyrazine Amine Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Pyrazole Pyrazine Amine Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. In one embodiment, the pharmaceutical composition is lactose-free. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Pyrazole Pyrazine Amine Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Pyrazole Pyrazine Amine Compound can be delayed or prolonged by proper formulation. For example, a slowly soluble pellet of the Pyrazole Pyrazine Amine Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Pyrazole Pyrazine Amine Compound in oily or emulsified vehicles that allow it to disperse slowly in the serum.

### 5. EXAMPLES

The following abbreviations were used in descriptions and examples:
ATP Adenosine triphosphate
Boc t-butyloxycarbonyl
BOP Benzotriazol-1-yloxy-tris-(dimethylamino) phosphonium hexafluorophosphate
CDI N,N′-carbonyldiimidazole
dba Dibenzylideneacetone
DCC Dicyclohexylcarbodiimide
DCM Dichloromethane
DEPBT (3-(Diethoxyphosphoryloxy)-1,2,3-benzotriazin-4(3H)-one)
DMAP 4-Dimethylaminopyridine
DMSO Dimethylsulfoxide
DPPA Diphenyl phosphorazidate
dppf 1,1′-Bis(diphenylphosphino)ferrocene
DTT Dithiothreitol
EDCI or 1-(3-Dimethylaminopropyl)-3-ethylcarbodiimide hydrochloride
EDC
EGTA Ethylene glycol tetraacetic acid
ESI Electrospray ionization
FBS Fetal bovine serum
FRET Fluorescence Resonance Energy Transfer
HATU 2-(7-Aza-1H-benzotriazole-1-yl)-1,1,3,3-tetramethyluronium hexafluorophosphate
HBTU 2-(1H-Benzotriazole-1-yl)-1,1,3,3-tetramethyluronium hexafluorophosphate
HCTU 2-(6-Chloro-1H-benzotriazole-1-yl)-1,1,3,3-tetramethylaminium hexafluorophosphate
HEPES (4-(2-Hydroxyethyl)-1-piperazineethanesulfonic acid
HOAt (1-Hydroxy-7-azabenzotriazole)
HOBt Hydroxybenzotriazole
HPLC High performance liquid chromatography
HTRF Homogeneous time resolved fluorescence
LC-MS Liquid chromatography mass spectrometry
LPS Lipopolysaccharide
LTMP Lithium 2,2,6,6-tetramethylpiperidide
MS Mass spectrometry
NMR Nuclear magnetic resonance
PyBOP Benzotriazol-1-yl-oxytripyrrolidinophosphonium hexafluorophosphate
PyBrOP Bromo-tris-pyrrolidinophosphonium-hexafluorophosphate
RPIMI Roswell Park Memorial Institute medium
rt Room temperature
TATU (O-(7-Azabenzotriazole-1-yl)-N,N,N′,N′-tetramethyluronium tetrafluoroborate
TBTU O-(1H-benzotriazole-1-yl)-N,N,N′,N′-tetramethyluronium tetrafluoroborate
TDBTU N,N,N′,N′-Tetramethyl-O-(3,4-dihydro-4-oxo-1,2,3-benzotriazin-3-yl)uranium tetrafluoroborate
TFA Trifluoracetic acid
THF Tetrahydrofuran
TR-FRET Time-resolved—Fluorescence Resonance Energy Transfer
TSTU O—(N-Succinimidyl)-1,1,3,3-tetramethyl uranium tetrafluoroborate

Compounds are named using the automatic name generating tool provided in Chemdraw Ultra 9.0 (Cambridgesoft), which generates systematic names for chemical structures, with support for the Cahn-lngold-Prelog rules for stereochemistry.

The following Examples are presented by way of illustration, not limitation.

### 5.1 Synthetic Examples

The compounds provided in this section were synthesized based on the general procedures provided in Schemes 1-5, Section 4.3, above.

### Example 1

### N²-(5-METHYL-1H-PYRAZOL-3-YL)-N⁶-PHENYLPYRAZINE-2,6-DIAMINE

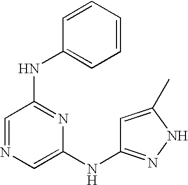

A. 6-Chloro-N-phenylpyrazin-2-amine. 2,6-Dichloropyrazine (510 mg, 3.4 mmol), palladium acetate (75 mg, 0.33 mmol), 4,5-bis(diphenylphosphino)-9,9-dimethylxanthene (405 mg, 0.7 mmol), potassium carbonate (4.6 g, 33.3 mmol), and aniline (0.3 mL, 3.3 mmol) were suspended in anhydrous dioxane (23 mL). The resulting mixture was degassed with nitrogen for 5 minutes and stirred at 90° C. for one hour. The reaction was partitioned between ethyl acetate and water. The aqueous layer was extracted with ethyl acetate (3×50 mL), and the organic layers were combined and dried over magnesium sulfate. After filtration, the solvent was removed in vacuo. The residue was purified by silica gel chromatography (0-60% ethyl acetate in hexanes) to give the title compound as a white solid (88 mg, 0.43 mmol, 13% yield); ¹H NMR (DMSO-d₆) δ 9.86 (s, 1H), 8.17 (s, 1H), 7.98 (s, 1H), 7.63 (d, J=10.0 Hz, 2H), 7.35 (t, J=7.2 Hz, 2H), 7.03 (t, J=6.8 Hz, 1H); MS (ESI) MS (ESI) m/z 206.0 [M+1]⁺.

B. tert-Butyl 5-methyl-3-{[6-(phenylamino)pyrazin-2-yl]amino}pyrazole-carboxylate. 6-Chloro-N-phenylpyrazin-2-amine (84 mg, 0.4 mmol), tert-butyl 3-amino-5-methyl-1H-pyrazole-1-carboxylate (98 mg, 0.5 mmol), palladium acetate (10 mg, 0.04 mmol), 4,5-bis(diphenylphosphino)-9,9-dimethylxanthene (55 mg, 0.1 mmol), and potassium carbonate (560 mg, 4.0 mmol) were suspended in anhydrous dioxane (3 mL). The resulting mixture was degassed with nitrogen for 5 minutes and stirred at 90° C. for one hour. The reaction was then partitioned between ethyl acetate and water. The aqueous layer was extracted with ethyl acetate (3×20 mL), and the organic layers were combined and dried over magnesium sulfate. After filtration, the solvent was removed in vacuo. The residue was purified by silica gel chromatography (15-75% ethyl acetate in hexanes) to give the title compound as an off-white solid (68 mg, 0.19 mmol, 46% yield); ¹H NMR (DMSO-d₆) δ 9.45 (s, 1H), 9.33 (s, 1H), 7.81 (s, 1H), 7.71 (s, 1H), 7.52 (d, J=8.4 Hz, 2H), 7.32 (t, J=8.4 Hz, 2H), 7.01 (t, J=7.6 Hz, 1H), 6.53 (s, 1H), 2.13 (s, 3H), 1.57 (s, 9H); MS (ESI) MS (ESI) m/z 367.3 [M+1]⁺.

C. N²-(5-Methyl-1H-pyrazol-3-yl)-N-phenylpyrazine-2,6-diamine. To a solution of tert-butyl 5-methyl-3-{[6-(phenylamino)pyrazin-2-yl]amino}pyrazole-carboxylate (68 mg, 0.19 mmol) in dichloromethane (8 mL) was added 4N HCl in dioxane (1.5 mL). The reaction was stirred at room temperature for one hour and the solvents were removed in vacuo. The crude solid was purified by reverse-phase preparative HPLC (20-80% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a slightly yellow solid (22 mg, 0.08 mmol, 46% yield); m.p. 204-205° C.;

¹H NMR (DMSO-d₆) δ 11.84 (s, 1H), 9.19 (s, 1H), 9.10 (s, 1H), 7.91 (s, 1H), 7.67 (d, J=7.2 Hz, 2H), 7.52 (s, 1H), 7.27 (t, J=7.6 Hz, 2H), 6.93 (t, J=7.2 Hz, 1H), 6.10 (s, 1H), 2.20 (s, 3H); MS (ESI) MS (ESI) m/z 267.3 [M+1]⁺.

### Example 2

### 3-(6-(5-METHYL-1H-PYRAZOL-3-YLAMINO)PYRAZIN-2-YLAMINO)BENZONITRILE

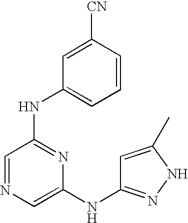

A. 3-(6-Chloropyrazin-2-ylamino)benzonitrile. 2,6-Dichloropyrazine (410 mg, 2.75 mmol), 3-aminobenzonitrile (390 mg, 3.3 mmol), Pd₂ dba₃ (38 mg, 1.5 mol %), 2-(dicyclohexylphosphino)biphenyl (30 mg, 3 mol %), and potassium phosphate (820 mg, 3.86 mmol) were suspended in dimethoxy ethane (6 mL). The resulting mixture was stirred in a sealed flask at 95° C. for 2 hours. The reaction mixture was cooled, diluted with ethyl acetate (50 mL), washed with water (50 mL), and then with brine (30 mL). The organic layer was dried (sodium sulfate), filtered, and concentrated. The residue was purified by silica gel chromatography (20-35% ethylacetate in hexanes). Fractions containing product were concentrated to give the title compound as an off-white solid (0.34 g, 54% yield); ¹H NMR (300 MHz, CDCl₃) δ 8.11 (s, 1H), 8.09 (s, 1H), 7.89 (t, J=2.1 Hz, 1H), 7.20 (dqJ₁=8.1 Hz, J₂=1.2 Hz, 1H,), 7.47 (t, J=7.5 Hz, 1H), 7.40 (dt, J=7.2 Hz, J2=1.5 Hz, 1H), 6.73 (br s, 1H).

B. 3-(6-(5-Methyl-1H-pyrazol-3-ylamino)pyrazin-2-ylamino)benzonitrile. 3-(6-chloropyrazin-2-ylamino)benzonitrile (0.32 g, 1.39 mmol), tert-butyl 3-amino-5-methyl-1H-pyrazole-1-carboxylate (0.3 g, 1.53 mmol), palladium acetate (31 mg, 10 mol %), (9,9-dimethyl-9H-xanthene-4,5-diyl)bis(diphenylphosphine) (160 mg, 20 mol %), and potassium carbonate (0.96 g, 6.95 mmol) were suspended in dioxane (8 mL). The mixture was stirred in a sealed flask at 90° C. for 1 hour. The reaction mixture was cooled, diluted with ethyl acetate (50 mL), washed with water (50 mL), and then with brine (30 mL). The organic layer was dried (sodium sulfate), filtered, and concentrated. The crude residue was dissolved in chloroform (20 mL) and 4N HCl in dioxane (3 mL) was added. The mixture was stirred at room temperature for 2 hours and the solvent evaporated. The crude product was purified by reverse-phase preparative HPLC (10-70% acetonitrile+0.1% TFA in H₂O+0.1% TFA, over 30 min). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a white solid (0.29 g, 72% yield); m.p. 260-262° C.; ¹H NMR (300 MHz, DMSO-d₆) δ 11.89 (br s, 1H), 9.50 (s, 1H), 9.38 (s, 1H), 8.34 (s, 1H), 7.89 (s, 1H), 7.69 (dd, J=8.4 Hz, J₂=1.2 Hz, 1H), 7.52 (s, 1H), 7.43 (t, J=8.4 Hz, 1H), 7.31 (d, J=7.6 Hz, 1H), 6.05 (s, 1H), 2.21 (s, 3H); MS (ESI) MS (ESI) m/z 292.0 [M+1]⁺.

### Example 3

### N²—CYCLOHEXYL-N⁶-(5-METHYL-1H-PYRAZOL-3-YL)PYRAZINE-2,6-DIAMINE

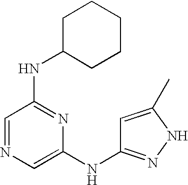

A. (6-Chloropyrazin-2-yl)cyclohexylamine. 2,6-Dichloropyrazine (570 mg, 3.8 mmol), dioxane (7 mL), cyclohexylamine (0.5 mL, 4.4 mmol) and N,N-diisopropylethylamine (0.8 mL, 4.6 mmol) were combined and heated to reflux for two days. The solvents were evaporated, and the residue was purified by silica gel chromatography (0-60% ethylacetate in hexanes). Fractions containing product were concentrated to give the title compound as a white solid (147 mg, 0.69 mmol, 18% yield);

¹H-NMR (DMSO-d₆) δ 7.84 (s, 1H), 7.63 (s, 1H), 7.44 (d, J=7.6 Hz, 1H), 3.64-3.59 (m, 1H), 1.90-1.86 (m, 2H), 1.73-1.68 (m, 2H), 1.60-1.56 (m, 1H), 1.37-1.16 (m, 5H); MS (ESI) MS (ESI) m/z 212.1 [M+1]⁺.

B. N²-cyclohexyl-N⁶-(5-methyl-1H-pyrazol-3-yl)pyrazine-2,6-diamine.

The title compound was prepared using the procedures substantially similar to those described in Example 1, steps B and C; m.p. 194-196° C.; ¹H NMR (DMSO-d₆) δ 11.73 (s, 1H), 8.96 (s, 1H), 7.48 (s, 1H), 7.17 (s, 1H), 6.51 (d, J=5.6 Hz, 1H), 6.26 (s, 1H), 3.62 (br s, 1H), 2.18 (s, 3H), 1.98-1.95 (m, 2H), 1.77-1.73 (m, 2H), 1.64-1.61 (m, 1H), 1.38-1.15 (m, 5H); MS (ESI) MS (ESI) m/z 273.4 [M+1]⁺.

### Example 4

### N²-(4-CHLOROPHENYL)-N⁶-(5-METHYL-1H-PYRAZOL-3-YL)PYRAZINE-2,6-DIAMINE

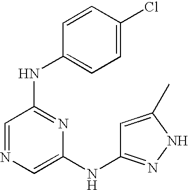

A. tert-Butyl 3-[(6-chloropyrazin-2-yl)amino]-5-methylpyrazolecarboxylate. 2,6-Dichloropyrazine (3.30 g, 22.2 mmol), palladium acetate (490 mg, 2.15 mmol), 4,5-bis(diphenylphosphino)-9,9-dimethylxanthene (2.64 g, 4.57 mmol), potassium carbonate (30 g, 217 mmol), and 1-boc-3-amino-5-methyl-pyrazole (4.35 mg, 22.0 mmol) were suspended in anhydrous dioxane (150 mL). The resulting mixture was degassed with nitrogen for 5 minutes and stirred at 90° C. for two hour. The reaction was partitioned between ethyl acetate and water. The aqueous layer was extracted with ethyl acetate and the organic layers were combined and dried over magnesium sulfate. After filtration, the solvent was removed in vacuo. The residue was purified by silica gel chromatography to give the title compound as a white solid (3.44 g, 11.1 mmol, 50% yield);

¹H NMR (DMSO-d₆) δ 10.03 (s, 1H), 8.54 (s, 1H), 8.18 (s, 1H), 6.61 (s, 1H), 2.20 (s, 3H), 1.57 (s, 9H); MS (ESI) MS (ESI) m/z 310.3 [M+1]⁺.

B. tert-Butyl 3-(6-(4-chlorophenylamino)pyrazin-2-ylamino)-5-methyl-1H-pyrazole-1-carboxylate. tert-Butyl 3-[(6-chloropyrazin-2-yl)amino]-5-methylpyrazolecarboxylate (305 mg, 1.00 mmol), 4-chloroaniline (160 mg, 1.25 mmol), palladium acetate (25 mg, 0.1 mmol), 4,5-bis(diphenylphosphino)-9,9-dimethylxanthene (138 mg, 0.25 mmol), and potassium carbonate (1.4 g, 10 mmol) were suspended in anhydrous dioxane (10 mL). The resulting mixture was degassed with nitrogen for 5 minutes and stirred at 90° C. for one hour. The reaction was then partitioned between ethyl acetate and water. The aqueous layer was extracted with ethyl acetate and the organic layers were combined and dried over magnesium sulfate. After filtration, the solvent was removed in vacuo. The residue was purified by silica gel chromatography to give the title compound as an off-white solid (186 mg, 0.46 mmol, 46% yield); ¹H NMR (DMSO-d₆) δ 9.47 (d, J=3.2 Hz, 1H), 7.84 (s, 1H), 7.01 (s, 1H), 7.54 (d, J=8.8 Hz, 2H), 7.32 (t, J=9.2 Hz, 2H), 6.44 (s, 1H), 2.15 (s, 3H), 1.56 (s, 9H); MS (ESI) MS (ESI) m/z 401.2 [M+1]⁺.

C. N²-(4-chlorophenyl)-N⁶-(5-methyl-1H-pyrazol-3-yl)pyrazine-2,6-diamine. To a solution of tert-butyl 3-(6-(4-chlorophenylamino)pyrazin-2-ylamino)-5-methyl-1H-pyrazole-1-carboxylate (182 mg, 0.45 mmol) in dichloromethane (15 mL) was added 4N HCl in dioxane (3 mL). The reaction was stirred at room temperature for two hour and the solvents were removed in vacuo. The crude solid was purified by reverse-phase preparative HPLC (20-80% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a slightly yellow solid (85 mg, 0.29 mmol, 62% yield);m.p. 227-228° C.; ¹H NMR (DMSO-d₆) δ 11.87 (s, 1H), 9.26 (s, 1H), 9.22 (s, 1H), 7.95 (s, 1H), 7.73 (d, J=8.8 Hz, 2H), 7.51 (s, 1H), 7.28 (d, J=8.8 Hz, 2H), 6.02 (s, 1H), 2.21 (s, 3H); MS (ESI) MS (ESI) m/z 301.2 [M+1]⁺.

### Example 5

### N²-(5-METHYL-1H-PYRAZOL-3-YL)-N′-(1,2,3,4-TETRAHYDROISOQUINOLIN-7-YL)PYRAZINE-2,6-DIAMINE

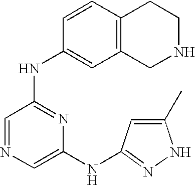

A. N²-(5-methyl-1H-pyrazol-3-yl)-N-6-(1,2,3,4-tetrahydroquinolin-7-yl)pyrazine-2,6-diamine. 4,5-bis(diphenylphosphino)-9,9-dimethylxanthene (112 mg, 0.19 mmol), tert-butyl 7-amino-3,4-dihydroisoquinoline-2(1H)-carboxylate (120 mg, 0.483 mmol) and tert-butyl 3-[(6-chloropyrazin-2-yl)amino]-5-methylpyrazolecarboxylate (150 mg, 0.483 mmol) were dissolved in anhydrous dioxane (1 mL) in a microwave-safe tube. To the resulting mixture was added potassium carbonate (334 mg, 2.42 mmol). Nitrogen was bubbled through the mixture for 1 min and palladium(II) acetate (22 mg, 0.097 mmol) was added followed by 2 drops of DMSO. The suspension was heated in the microwave reactor for 40 min at 80° C. The mixture was filtered through Celite, washed with methylene chloride and concentrated. The crude material was dissolved in dichloromethane (20 mL) and TFA was added (2 mL) was added. The reaction was stirred at room temperature for 3 hours and concentrated. The crude solid was purified by reverse-phase preparative HPLC (10-50% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a solid (63 mg, 0.196 mmol, 41% yield); MS (ESI) MS (ESI) m/z 322.5 [M+1]⁺.

### Example 6

### N²-(2-METHYL-1,2,3,4-TETRARYDROISOQUINOLIN-7-YL)-N⁶-(5-METHYL-1H-PYRAZOL-3-YL)PYRAZINE-2,6-DIAMINE

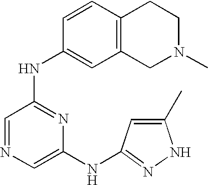

A. N²-(2-methyl-1,2,3,4-tetrahydroisoquinolin-7-yl)-N⁶-(5-methyl-1H-pyrazol-3-yl)pyrazine-2,6-diamine. tert-Butyl 7-(6-(5-methyl-1H-pyrazol-3-ylamino)pyrazin-2-ylamino)-3,4-dihydroisoquinoline-2(1H)-carboxylate (632 mg, 1.5 mmol) was dissolved in tetrahydrofuran (5 mL) and cooled to 0° C. under nitrogen. Lithium aluminum hydride (0.570 g, 15 mmol) was added and the reaction was warmed to rt over 1 h. After heating at 40° C. for 7 h, the reaction was cooled to 0° C. and ammonium chloride (aq, saturated) was added carefully with stirring. The crude reaction was diluted with water (10 mL), extracted with methylene chloride (3×, 100 mL), dried and concentrated in vacuo. The crude solid was purified by reverse-phase preparative HPLC (10-40% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a solid (48 mg, 4.4% yield); mp 152-156; ¹H NMR (400 MHz, DMSO-d₆) δ ppm 11.86 (s, 1H), 9.21 (s, 1H), 8.97 (s, 1H), 7.75 (s, 1H), 7.53 (s, 1H), 7.45 (s, 1H), 7.06-7.28 (m, 1H), 7.00 (d, J=8.20 Hz, 1H), 6.17 (s, 1H), 3.44 (s, 2H), 2.68-2.81 (m, 2H), 2.54-2.62 (m, 2H), 2.33 (s, 3H), 2.21 (s, 3H); MS (ESI) MS (ESI) m/z 336.1 [M+1]⁺.

### Example 7

### N-(2-METHYL-5-(6-(5-METHYL-1H-PYRAZOL-3-YLAMINO)PYRAZIN-2-YLAMINO)PHENYL)-2-(METHYLAMINO)ACETAMIDE

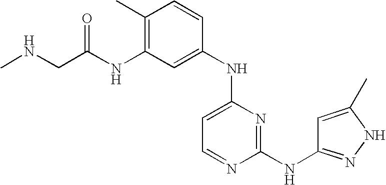

A. tert-Butyl 2-(5-bromo-2-methylphenylamino)-2-oxoethyl(methyl)carbamate. To a solution of 5-bromo-2-methylaniline (0.744 g, 4 mmol), DMAP (0.538 g, 4.40 mmol) and 2-(tert-butoxycarbonyl(methyl)amino)acetic acid (0.833 g, 4.40 mmol) in DMF (10 mL) was added EDC (0.843 g, 4.40 mmol). The reaction mixture was stirred at 25° C. for 15 h, concentrated, and purified by silica gel chromatography (15% ethylacetate in hexanes). Fractions containing product were concentrated to give the title compound as a white solid (1.318 g, 3.69 mmol, 92% yield); MS (ESI) MS (ESI) m/z 359.1 [M+1]⁺.

B. tert-Butyl 2-(5-bromo-2-methylphenylamino)-2-oxoethyl(methyl)carbamate. tert-Butyl 2-(5-bromo-2-methylphenylamino)-2-oxoethyl(methyl)carbamate (1.31 g, 3.67 mmol), 6-chloropyrazin-2-amine (0.523 g, 4.03 mmol), Xantphpos (0.212 g, 0.367 mmol), palladium acetate (0.041 g, 0.183 mmol), and potassium carbonate (2.53 g, 18.33 mmol) were suspended in 1,4-dioxane (10 mL). The reaction mixture was stirred at 85° C. for 4 h. The reaction was partitioned between ethyl acetate and aqueous sodium chloride. The aqueous layer was extracted with ethyl acetate (3×25 mL), and the organic layers were combined and dried over magnesium sulfate. After filtration, the solvent was removed in vacuo. The residue was purified by silica gel chromatography (30-55% ethyl acetate in hexanes) to give the title compound as a white solid (0.3873 g, 0.954 mmol, 26.0% yield)); MS (ESI) MS (ESI) m/z 406.1 [M+1]⁺.

C. N-(2-Methyl-5-(6-(5-methyl-1H-pyrazol-3-ylamino)pyrazin-2-ylamino)phenyl)-2-(methylamino)acetamide. tert-Butyl 2-(5-(6-chloropyrazin-2-ylamino)-2-methylphenylamino)-2-oxoethyl(methyl)carbamate (0.387 g, 0.954 mmol) was converted to the title compound using the procedures substantially similar to those described in Example 1 step B followed by step C as white solid (0.040 g, 0.109 mmol, 11% yield); ¹H NMR (400 MHz, DMSO-d₆) δ 11.82 (br s, 1H), 9.46 (br s, 1H), 9.17 (br s, 1H), 9.07 (br, s, 1H), 7.73-7.96 (m, 2H), 7.64 (br. s., 1H), 7.52 (s, 1H), 7.11 (d, J=8.20 Hz, 1H), 6.12 (br s, 1H), 3.23 (s, 2H), 2.36 (s, 3H), 2.19 (s, 6H); MS (ESI) MS (ESI) m/z 367.2 [M+1]⁺.

### Example 8

### METHYL 5-(6-(5-METHYL-1H-PYRAZOL-3-YLAMINO)PYRAZIN-2-YLAMINO)-2-(TRIFLUOROMETHYL)PHENYLCARBAMATE

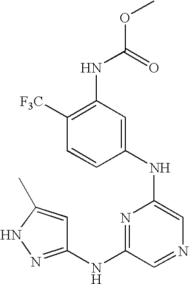

A. 5-Bromo-2-(trifluoromethyl)benzoate. To a solution of methyl 5-bromo-2-iodobenzoate (4.65 g, 13.64 mmol) and methyl 2,2-difluoro-2-(fluorosulfonyl)acetate (2.6 mL, 20.44 mmol) in N-methyl-2-pyrrolidinone (10 mL) was added copper(I) bromide (235 mg, 1.638 mmol). The reaction mixture was stirred at 120° C. for 15 h in a sealed tube. The reaction was partitioned between ethyl acetate and aqueous sodium chloride. The aqueous layer was extracted with ethyl acetate (3×25 mL), and the organic layers were combined and dried over magnesium sulfate. After filtration, the solvent was removed in vacuo. The residue was purified by silica gel chromatography (1-5% ethyl acetate in hexanes) to give the title compound as an oil (2.9 g, 10.25 mmol, 75% yield); ¹H NMR (400 MHz, DMSO-d₆) δ 8.08 (s, 1H), 8.01 (d, J=8.20 Hz, 1H), 7.83 (d, J=8.20 Hz, 1H), 3.90 (s, 3H).

B. 5-Bromo-2-(trifluoromethyl)benzoic acid. To a solution of methyl 5-bromo-2-(trifluoromethyl)benzoate (4.2 g, 14.8 mmol) in methanol (10 mL) was added sodium hydroxide (10 mL, 50.0 mmol). After stirring at 55° C. for 2 h, saturated aqueous sodium chloride (25 mL) was added and the pH adjusted to 5 by addition of hydrochloric acid (50.2 mL). The aqueous layer was extracted with ethyl acetate (3×25 mL), and the organic layers were combined and dried over magnesium sulfate. After filtration, the solvent was removed in vacuo. The residue was purified by silica gel chromatography (10-55% ethyl acetate in hexanes) to give the title compound as a solid (3.76 g, 13.98 mmol, 94% yield); ¹H NMR (400 MHz, DMSO-d₆) δ 13.93 (br. s., 1H), 8.02 (d, J=1.95 Hz, 1H), 7.93-7.98 (m, 1H), 7.79 (d, J=8.20 Hz, 1H).

C. 5-Bromo-2-(trifluoromethyl)phenylcarbamate. A solution of 5-bromo-2-(trifluoromethyl)benzoic acid (3.76 g, 13.98 mmol) in thionyl chloride (3 mL, 41.1 mmol) was stirred at 85° C. for 2 h. Excess thionyl chloride was removed under reduced pressure. The residue was dissolved in 1,4-dioxane (6 mL) and a solution of sodium azide (1.817 g, 28.0 mmol) in water (6 mL) was added dropwise at 0° C. After stirring for 0.5 hours, the reaction mixture was extracted with ethyl acetate (3×25 mL) and washed with saturated aqueous sodium chloride (3×25 mL). The organic layers were combined, dried (MgSO₄), filtered and concentrated under reduced pressure. The acyl azide was dissolved in toluene (10 mL) and heated to 80° C. with stirring. After 2 h, methanol (0.679 mL, 16.77 mmol) was added and stirring at 85° C. was continued for 16 h. The reaction mixture was concentrated and the crude product purified by silica gel chromatography (10-50% ethyl acetate in hexanes) to give the title compound as a white solid (3.52 g, 11.8 mmol, 85% yield); ¹H NMR (400 MHz, DMSO-d₆) δ 9.27 (s, 1H), 7.80 (s, 1H), 7.65-7.67 (m, 2H), 3.66 (s, 3H).

D. Methyl 5-(6-(5-methyl-1H-pyrazol-3-ylamino)pyrazin-2-ylamino)-2-(trifluoromethyl)phenylcarbamate. Methyl 5-(6-chloropyrazin-2-ylamino)-2-(trifluoromethyl)phenylcarbamate (347 mg, 1 mmol) was synthesized from 5-bromo-2-(trifluoromethyl)phenylcarbamate and 6-chloropyrazin-2-amine and converted to the title compound using the procedures substantially similar to those described in Example 7 steps B and C as a white solid. (150 mg, 0.368 mmol, 36.8% yield); m.p. 203® C.-204° C.; ¹H NMR (400 MHz, DMSO-d₆) δ 11.90 (s, 1H), 9.58 (s, 1H), 9.34 (s, 1H), 8.94 (s, 1H), 8.00 (s, 1H), 7.90 (d, J=8.98 Hz, 1H), 7.61-7.69 (m, 1H), 7.58 (s, 1H), 7.54 (d, J=8.59 Hz, 1H), 6.05 (s, 1H), 3.62 (s, 3H), 2.22 (s, 3H); MS (ESI) MS (ESI) m/z 408.1 [M+1]⁺.

### Example 9

### METHYL 5-(6-(5-METHYL-1H-PYRAZOL-3-YLAMINO)PYRAZIN-2-YLAMINO)-2-PROPYLPHENYLCARBAMATE

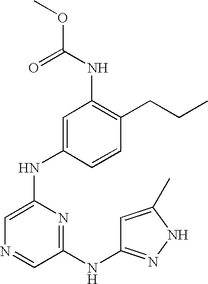

A. 3-Nitro-4-propylaniline. To a solution of 4-propylaniline (4.65 g, 34.4 mmol) in sulfuric acid (20 mL, 396 mmol) was added nitric acid (10 mL, 180 mmol) at 0° C. The reaction mixture was stirred at 0° C. for and allowed warm up to room temperature for 15 h. The reaction mixture was poured onto 200 g of ice and adjusted pH 8 by addition of 1N NaOH. The mixture was then extracted with ethyl acetate (3×25 mL) and washed with saturated aqueous NaCl (3×25 mL). The organic layers were combined, dried (MgSO₄), filtered, and concentrated. The residue was purified by silica gel chromatography (20-50% ethyl acetate in hexanes) to give the title compound as a brown oil (4.2 g, 23.31 mmol, 67.8% yield); MS (ESI) MS (ESI) m/z 181.1 [M+1]⁺.

B. tert-butyl 3-nitro-4-propylphenylcarbamate. To a solution of 3-nitro-4-propylaniline (0.451 g, 2.5 mmol), di-tert-butyl dicarbonate (0.546 g, 2.500 mmol) in THF (5 mL) was added sodium hydroxide (2.50 mL, 5.0 mmol). After stirring at 45° C. for 15 h, saturated aqueous sodium chloride (25 mL) was added and the mixture was extracted with ethyl acetate (3×25 mL). The organic layers were combined, dried (MgSO₄), filtered, and concentrated. The residue was purified by silica gel chromatography (25% ethyl acetate in hexanes) to give the title compound (0.5 g, 1.784 mmol, 71.3% yield).

C. tert-Butyl 2-(3-amino-4-propylphenyl)acetate. To a solution of tert-butyl 2-(3-nitro-4-propylphenyl)acetate (0.50 g, 1.790 mmol) in methanol (10 mL) was added palladium on carbon (0.095 g, 0.089 mmol). The reaction mixture was stirred at 25° C. for 1 h under hydrogen at 45 psi, filtered over celite, and concentrated. The crude product was purified by silica gel chromatography (20% ethyl acetate in hexanes) to give the title compound as a brown oil (0.400 g, 1.604 mmol, 90% yield); MS (ESI) MS (ESI) m/z 251.3 [M+1]⁺; ¹H NMR (400 MHz, DMSO-d₆) δ 8.92 (s, 1H), 6.82 (s, 1H), 6.72 (d, J=8.20 Hz, 1H), 6.50 (dd, J=1.95, 8.20 Hz, 1H), 4.73 (br. s., 2H), 2.28-2.36 (m, 2H), 1.35-1.53 (m, 11H), 0.89 (t, J=7.22 Hz, 3H).

D. (tert-Butoxy)-N-[3-(methoxycarbonylamino)-4-propylphenyl]carboxamide. To a solution of tert-butyl 3-amino-4-propylphenylcarbamate (0.400 g, 1.598 mmol), N-ethyl-N-isopropylpropan-2-amine (0.248 g, 1.917 mmol) in THF (10 mL) was added methyl carbonochloridate (0.181 g, 1.917 mmol). After stirring at 25° C. for 15 h, saturated aqueous sodium chloride (25 mL) was added and the mixture was extracted with ethyl acetate (3×25 mL). The organic layers were combined, dried (MgSO₄), filtered, and concentrated. The residue was purified by silica gel chromatography (20% ethyl acetate in hexanes) to give the title compound as a brown oil (0.324 g, 1.051 mmol, 65.8% yield); MS (ESI) MS (ESI) m/z 309.5 [M+1]⁺; ¹H NMR (400 MHz, DMSO-d₆) δ 9.26 (s, 1H), 8.77 (s, 1H), 7.47 (br. s., 1H), 7.14 (dd, J=1.95, 8.20 Hz, 1H), 7.03 (d, J=8.20 Hz, 1H), 3.62 (s, 3H), 1.37-1.50 (m, 11H), 0.85 (t, J=7.42 Hz, 3H).

E. 5-Amino-2-propylphenylcarbamate. To a solution of tert-butyl 3-amino-4-propylphenylcarbamate (0.324 g, 1.294 mmol) in dioxane (5 mL) was added hydrogen chloride (1 mL, 4.00 mmol). After stirring at 55° C. for 1 h, saturated aqueous sodium chloride (25 mL) was added and the mixture was extracted with ethyl acetate (3×25 mL). The organic layers were combined, dried (MgSO₄), filtered, and concentrated. The residue was purified by silica gel chromatography (50% ethyl acetate in hexanes) to give the title compound as a brown oil (0.156 g, 0.749 mmol, 57.9% yield); MS (ESI) m/z 209.1 [M+1]⁺.

F. Methyl 5-(6-(5-methyl-1H-pyrazol-3-ylamino)pyrazin-2-ylamino)-2-propylphenylcarbamate. To a suspension of methyl 5-amino-2-propylphenylcarbamate (0.156 g, 0.749 mmol), tert-butyl 3-(6-chloropyrazin-2-ylamino)-5-methyl-1H-pyrazole-1-carboxylate (0.278 g, 0.899 mmol), potassium carbonate (0.518 g, 3.75 mmol), Xantphos (0.087 g, 0.150 mmol) in 1,4-dioxane (5 mL) was added palladium(II) acetate (0.017 g, 0.075 mmol). The reaction mixture was stirred at 90° C. for 2 h. The reaction mixture was filtered and TFA (1.0 mL) was added. After stirring for 2 h, the reaction mixture was concentrated. The crude solid was purified by reverse-phase preparative HPLC (10-70% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and recrystallized in ethyl acetate/methanol (19:1) to give the title compound as an orange solid (55 mg, 0.144 mmol, 19.21% yield); ¹H NMR (400 MHz, DMSO-d₆) δ 11.82 (s, 1H), 9.18 (s, 1H), 9.02 (s, 1H), 8.79 (s, 1H), 7.83 (s, 1H), 7.55 (d, J=8.59 Hz, 1H), 7.48 (d, J=8.98 Hz, 2H), 7.06 (d, J=8.59 Hz, 1H), 6.14 (br. s., 1H), 3.62 (s, 3H), 2.45 (m, 2H), 2.19 (s, 3H), 1.50 (sxt, J=7.42 Hz, 2H), 0.89 (t, J=7.42 Hz, 3H); MS (ESI) m/z 382.2 [M+1]⁺.

### Example 10

### 2-CHLORO-N,N-DIMETHYL-5-(6-(5-METHYL-1H-PYRAZOL-3-YLAMINO)PYRAZIN-2-YLAMINO)BENZAMIDE

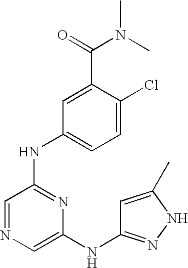

A. 2-Chloro-5-(6-(5-methyl-1H-pyrazol-3-ylamino)pyrazin-2-ylamino)benzoic acid. tert-Butyl 3-(6-(4-chloro-3-(methoxycarbonyl)phenylamino)pyrazin-2-ylamino)-5-methyl-1H-pyrazole-1-carboxylate (400 mg, 1.12 mmol) was added to a mixture of potassium hydroxide (300 mg, 5.35 mmol), water (7.5 mL) and ethanol (7.5 mL). The reaction was stirred at room temperature for 4 h. The solution was acidified to pH 4 with acetic acid and the product was collected by filtration to give 2-chloro-5-(6-(5-methyl-1H-pyrazol-3-ylamino)pyrazin-2-ylamino)benzoic acid (300 mg, 0.870 mmol, 78% yield); MS (ESI) m/z 345.2 [M+1]⁺.

B. 2-Chloro-N,N-dimethyl-5-(6-(5-methyl-1H-pyrazol-3-ylamino)pyrazin-2-ylamino)benzamide. 2-Chloro-5-(6-(5-methyl-1H-pyrazol-3-ylamino)pyrazin-2-ylamino)benzoic acid (212 mg, 0.615 mmol) and dimethylamine hydrochloride (150 mg, 1.839 mmol) were combined in dimethylformamide (4 mL) and diisopropylethylamine (214 μl, 1.225 mmol). HATU (280 mg, 0.736 mmol) was added and the mixture was stirred at room temperature for 1 h. The reaction mixture was purified by reverse-phase preparative HPLC (10-70% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a solid (30 mg, 0.081 mmol, 13.12% yield); m.p. 213-215° C.; ¹H NMR (400 MHz, DMSO-d₆) δ 11.87 (br. s., 1H), 9.35 (s, 1H), 9.29 (s, 1H), 7.93 (s, 1H), 7.76 (d, J=8.98 Hz, 1H), 7.59-7.65 (m, 1H), 7.52 (s, 1H), 7.37 (d, J=8.98 Hz, 1H), 6.02 (s, 1H), 3.00 (s, 3H), 2.79 (s, 3H), 2.22 (s, 3H); MS (ESI) m/z 372.1 [M+1]⁺.

### Example 11

### N-(3-(6-(5-METHYL-1H-PYRAZOL-3-YLAMINO)PYRAZIN-2-YLAMINO)PHENYL)BENZAMIDE

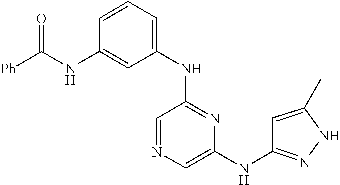

A. tert-Butyl 5-methyl-3-(6-(3-nitrophenylamino)pyrazin-2-ylamino)-1H-pyrazole-1-carboxylate. The title compound was prepared using the procedures substantially similar to those described in Example 1, steps A and B; ¹H NMR (300 MHz, CDCl₃) δ 9.89 (s, 1H), 8.47 (t, 1H, J=2.1 Hz), 7.93 (ddd, J, =8.4 Hz, J₂=2.7 Hz, J₃=0.9 Hz, 1H), 7.81 (s, 1H), 7.78 (s, 1H), 7.69 (ddd, J, =7.8 Hz, J₂=3.0 Hz, J₃=0.6 Hz, 1H), 7.50 (t, J=8.1 Hz, 1H), 6.91 (s, 1H), 6.52 (s, 1H), 2.05 (s, 3H), 1.69 (s, 9H).

B. tert-Butyl 3-(6-(3-aminophenylamino)pyrazin-2-ylamino)-5-methyl-1H-pyrazole-1-carboxylate. tert-Butyl 5-methyl-3-(6-(3-nitrophenylamino)pyrazin-2-ylamino)-1H-pyrazole-1-carboxylate (1.43 g, 3.48 mmol) was dissolved in ethanol (30 mL) and 10% Pd/C (0.48 g) was added. The mixture was purged with H₂(g) and stirred under H₂ at atmospheric pressure at room temperature for one hour. The mixture was filtered through a pad of Celite and the filtrate concentrated to afford the title compound (1.32 g, 3.48 mmol, 100% yield); MS (ESI) m/z 382.1 [M+1]⁺.

C. N-(3-(6-(5-methyl-1H-pyrazol-3-ylamino)pyrazin-2-ylamino)phenyl)benzamide. To a solution of tert-butyl 3-(6-(3-aminophenylamino)pyrazin-2-ylamino)-5-methyl-1H-pyrazole-1-carboxylate (0.2 g, 0.53 mmol) in pyridine (2 mL), was added benzoyl chloride (0.2 mL). After stirring at room temperature for 2 hours, the mixture was poured in ethyl acetate (30 mL) and washed with 5% HCl (30 mL), H₂O (30 mL), and then brine (20 mL). The organic layer was dried (sodium sulfate), filtered, and concentrated to approximately 15 mL of volume. To this solution was added 4N HCl in dioxane (3 mL). This mixture was stirred for 30 minutes and the solvents were removed in vacuo. The crude solid was purified by reverse-phase preparative HPLC (20-70% acetonitrile+0.1% TFA in H₂O+0.1% TFA, over 30 min.). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a tan solid (50 mg, 30% yield); m.p. 243-244° C.; ¹H NMR (400 MHz, DMSO) δ 11.81 (s, 1H), 10.19 (s, 1H), 9.20 (br s, 1H), 9.16 (s, 1H), 7.94 (m, 4H), 7.87 (br s, 1H), 7.51-7.62 (m, 5H), 7.35 (d, J=8.0 Hz, 1H), 7.25 (t, J=8.0 Hz, 1H), 6.16 (br s, 1H), 2.11 (s, 3H); MS (ESI) m/z 387.5 [M+1]⁺.

### Example 12

### N²-(4-CHLORO-3-(2-(DIMETHYLAMINO)ETHOXY)PHENYL)-N⁶-(5-METHYL-1H-PYRAZOL-3-YL)PYRAZINE-2,6-DIAMINE

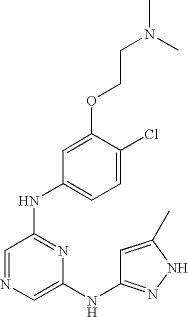

A. N²-(4-chloro-3-(2-(dimethylamino)ethoxy)phenyl)-N⁶-(5-methyl-1H-pyrazol-3-yl)pyrazine-2,6-diamine. tert-Butyl 3-(6-(4-chloro-3-hydroxyphenylamino)pyrazin-2-ylamino)-5-methyl-1H-pyrazole-1-carboxylate (200 mg, 0.480 mmol), 2-chloro-N,N-dimethylethanamine hydrochloride (138 mg, 0.960 mmol) and cesium carbonate (625 mg, 1.919 mmol) were combined in dimethyl formamide (3 mL). The mixture was stirred at room temperature for 18 h. The solids were filtered off and washed with ethyl acetate. The filtrate was evaporated and the crude material was stirred with 1:1 TFA/DCM (15 mL) for 1 h. The reaction mixture was purified by reverse-phase preparative HPLC (20-70% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as a solid (22 mg, 0.057 mmol, 11.82% yield); m.p. 145-147° C.;

¹H NMR (400 MHz, DMSO-d₆) δ 11.89 (br. s., 1H), 9.14-9.30 (m, 2H), 7.89 (s, 1H), 7.51 (s, 1H), 7.43 (s, 1H), 7.25 (s, 2H), 6.06 (br. s., 1H), 4.03-4.08 (m, 2H), 2.63 (t, J=5.86 Hz, 2H), 2.22 (s, 6H), 2.19 (s, 3H); MS (ESI) m/z 387.1 [M+1]⁺.

### Example 13

### N-(5-methyl-1H-pyrazol-3-yl)-6-phenylpyrazin-2-amine

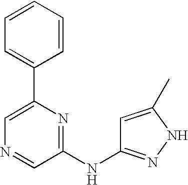

A. 2-Chloro-6-phenylpyrazine. 2,6-Dichloropyrazine (360 mg, 2.4 mmol), phenylboronic acid (295 mg, 2.4 mmol), tetrakis(triphenylphosphine)palladium(0) (290 mg, 0.25 mmol), potassium carbonate (333 mg, 2.4 mmol), ethanol (1 mL), and toluene (5 mL) were combined and the mixture degassed with nitrogen for 2 minutes before heating to 80° C. for one hour. The reaction was partitioned between ethyl acetate and water. The aqueous layer was washed with ethyl acetate (3×), the organics were combined, dried over magnesium sulfate, filtered, and concentrated. The crude material was purified by silica gel chromatography (0-30% ethyl acetate in hexanes) to give the title compound as a solid (108 mg, 0.56 mmol, 23% yield); MS (ESI) MS (ESI) m/z 191.2 [M+1]⁺.

B. tert-Butyl 5-methyl-3-(6-phenylpyrazin-2-ylamino)-1H-pyrazole-1-carboxylate. 2-Chloro-6-phenylpyrazine (108 mg, 0.56 mmol), tert-butyl 3-amino-5-methyl-1H-pyrazole-1-carboxylate (142 mg, 0.72 mmol), palladium(II) acetate (15 mg, 0.07 mmol), 4,5-bis(diphenylphosphino)-9,9-dimethylxanthene (65 mg, 0.1 mmol), potassium carbonate (820 mg, 5.9 mmol), and dioxane (4 mL) were combined and the mixture degassed with nitrogen for 2 minutes before heating to 90° C. for one hour. The reaction was partitioned between ethyl acetate and water. The aqueous layer was washed with ethyl acetate (3×), organics were combined, dried over magnesium sulfate, filtered, and concentrated. The crude material was purified by silica gel chromatography (10-65% ethyl acetate in hexanes) to give the title compound as a white solid (92 mg, 0.26 mmol, 46% yield); MS (ESI) MS (ESI) m/z 352.4 [M+1]⁺.

C. N-(5-Methyl-1H-pyrazol-3-yl)-6-phenylpyrazin-2-amine. tert-Butyl 5-methyl-3-(6-phenylpyrazin-2-ylamino)-1H-pyrazole-1-carboxylate (90 mg, 0.26 mmol) was dissolved in chloroform (8 mL) and 4N HCl in dioxane (2 mL) was added. The reaction mixture was stirred at room temperature for one hour and the solvents evaporated. The crude material was purified by reverse-phase preparative HPLC (30-100% acetonitrile+0.1% TFA in H₂O+0.1% TFA). Fractions containing product were condensed and neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound as an off-white solid ((21 mg, 0.08 mmol, 31% yield): m.p. 232-234° C.; ¹H-NMR (DMSO-d₆) δ 11.95 (s, 1H), 9.73 (s, 1H), 8.48 (s, 2H), 8.08 (d, J=6.9 Hz, 2H), 7.56-7.50 (m, 3H), 6.36 (s, 1H), 2.25 (s, 3H); MS (ESI) MS (ESI) m/z 252.4 [M+1]⁺.

### Example 14

### 6-(4-CHLOROPHENYL)-N-(5-METHYL-1H-PYRAZOL-3-YL)PYRAZIN-2-AMINE

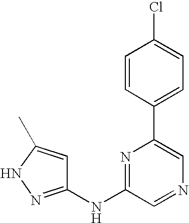

A. tert-Butyl 3-(6-chloropyrazin-2-ylamino)-5-methyl-1H-pyrazole-1-carboxylate. 2,6-Dichloropyrazine (5.0 g, 33.6 mmol), tert-butyl 3-amino-5-methyl-1H-pyrazole-1-carboxylate (6.62 g, 33.6 mmol), palladium(II) acetate (0.75 g, 3.36 mmol), 4,5-bis(diphenylphosphino)-9,9-dimethylxanthene (3.88 g, 6.71 mmol), potassium carbonate (46.4 g, 336 mmol), and dioxane (224 mL) were combined and the mixture was degassed with nitrogen for 2 minutes before heating to 90° C. for one hour. The reaction was cooled to room temperature and filtered through Celite followed by washing with dichloromethane. The filtrate was condensed and the crude material was purified by silica gel chromatography (10-70% ethyl acetate in hexanes) to give the title compound as a white solid (6.43 g, 20.8 mmol, 62% yield); MS (ESI) MS (ESI) m/z 310.5 [M+1]⁺.

B. 6-(4-chlorophenyl)-N-(5-methyl-1H-pyrazol-3-yl)pyrazin-2-amine. To solution of tert-butyl 3-(6-chloropyrazin-2-ylamino)-5-methyl-1H-pyrazole-1-carboxylate (200 mg, 0.65 mmol) and 4-chlorophenylboronic acid (116 mg, 0.74 mmol) in THF (4 mL) was added a solution of potassium carbonate (268 mg, 1.94 mmol) in water (1 mL). Palladium(II) acetate (7.25 mg, 0.032 mmol) and triphenylphosphine (16.9 mg, 0.065 mmol) were added and the mixture was heated at reflux overnight. The reaction was partitioned between dichloromethane and water. The aqueous layer was washed with dichloromethane (2×) and the organics were combined, condensed, and then acidified with 4M HCl in dioxane (2 mL) overnight. The reaction was condensed and the crude material was purified by reverse-phase preparative HPLC (20-100% acetonitrile+0.1% TFA in H₂O+0.1% TFA). Fractions containing product were condensed and neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound as an orange colored solid (75 mg, 0.26 mmol, 40% yield); MS (ESI) MS (ESI) m/z 286.4 [M+1]⁺.

### Example 15

### 1-(3-(4-FLUOROPHENYLAMINO)-5-(5-METHYL-1H-PYRAZOL-3-YLAMINO)PYRAZIN-2-YL)PROPAN-1-ONE

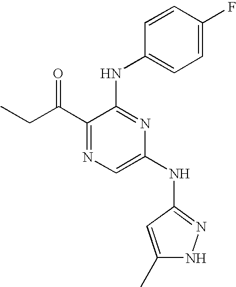

A. 1-(3,5-Dichloropyrazin-2-yl)propan-1-ol. To a solution of 2,2,6,6-tetramethylpiperidine (4.5 mL, 26.5 mmol) in dry THF (440 mL) at 0° C. was added n-butyllithium (1.6 M in hexanes, 16.5 mL, 26.4 mmol). The mixture was maintained at 0° C. for 15 minutes and then cooled to −90° C. 2,6-Dichloropyrazine (3.3 g, 22.2 mmol) in THF (20 mL) was added followed by propionaldehyde (2.5 mL, 34 mmol) in THF (10 mL). The reaction was stirred at −90° C. for 10 minutes and then poured onto H₂O (400 mL). The resulting mixture was extracted with diethyl ether (2×). The combined organic layers were dried (MgSO₄), filtered, and concentrated. The crude residue was purified by silica gel chromatography (0-60% ethyl acetate in hexanes) to give the desired product as a yellow oil (3.0 g, 14.5 mmol, 65%): ¹H-NMR (DMSO-d₆) δ 8.85 (s, 1H), 5.48 (d, J=6.4 Hz, 1H), 4.83 (q, J=6.4 Hz, 1H), 1.83-1.74 (m, 2H), 0.87 (t, J=7.6 Hz, 3H); MS (ESI) m/z 207 [M+1]⁺.

B. 1-(3,5-Dichloropyrazin-2-yl)propan-1-one. To a solution of 1-(3,5-dichloroprazin-2-yl)propan-1-ol (520 mg, 2.5 mmol) in dichloromethane (20 mL) was added Dess Martin periodinane (2.05 g, 4.8 mmol) in small portions. The reaction was stirred at room temperature for 30 minutes then filtered over Celite, washing with dichloromethane. The filtrate was concentrated and purified by silica gel chromatography (0-30% ethyl acetate in hexanes) to give the desired product as a white solid (518 mg, 2.5 mmol, 100%):

¹H-NMR (CDCl₃) δ 8.54 (s, 1H), 3.11 (q, J=7.2 Hz, 2H), 1.22 (t, J=7.2 Hz, 3H); MS (ESI) m/z 205.1 [M+1]⁺.

C. tert-Butyl 3-(6-chloro-5-propionylpyrazin-2-ylamino)-5-methyl-1H-pyrazole-1-carboxylate. 1-(3,5-Dichloropyrazin-2-yl)propan-1-one (366 mg, 1.79 mmol), tert-butyl 3-amino-5-methyl-1H-pyrazole-1-carboxylate (352 mg, 1.78 mmol), palladium(II) acetate (62 mg, 0.28 mmol), 4,5-bis(diphenylphosphino)-9,9-dimethylxanthene (312 mg, 0.54 mmol), and potassium carbonate (2.42 g, 17.5 mmol) were suspended in anhydrous dioxane (11 mL). The resulting mixture was degassed with nitrogen for 2 minutes and stirred at 50° C. for 30 minutes. The reaction was filtered through Celite, washing thoroughly with dichloromethane. The filtrate was concentrated and the crude residue was purified by silica gel chromatography (0-60% ethyl acetate in hexanes) to give the desired product as a red solid (481 mg, 1.31 mmol, 73%): ¹H-NMR (CDCl₃) δ 10.40 (s, 1H), 8.13 (s, 1H), 6.88 (s, 1H), 3.12 (q, J=7.2 Hz, 2H), 2.34 (s, 3H), 1.71 (s, 9H), 1.20 (t, J=7.2 Hz, 3H); MS (ESI) m/z 366.3 [M+1]⁺.

D. 1-(3-(4-Fluorophenylamino)-5-(5-methyl-1H-pyrazol-3-ylamino)pyrazin-2-yl)propan-1-one. tert-Butyl 3-(6-chloro-5-propionylpyrazin-2-ylamino)-5-methyl-1H-pyrazole-1-carboxylate (122 mg, 0.33 mmol), 4-fluoroaniline (0.05 mL, 0.52 mmol), palladium(II) acetate (12 mg, 0.053 mmol), 4,5-bis(diphenylphosphino)-9,9-dimethylxanthene (62 mg, 0.11 mmol), and potassium carbonate (513 mg, 3.7 mmol) were suspended in anhydrous dioxane (3 mL). The resulting mixture was degassed with nitrogen for 2 minutes and stirred at 50° C. for 30 minutes. The reaction was filtered through Celite, washing thoroughly with dichloromethane. The filtrate was concentrated and 4M HCl in dioxane (2 mL) was added and allowed to stir for 2 minutes. The mixture was concentrated and the crude residue was purified by reverse-phase preparative HPLC (30-100% acetonitrile+0.1% TFA in H₂O+0.1% TFA). Fractions containing product were condensed and neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound as a yellow solid (32 mg, 0.094 mmol, 28%): m.p. 208-210° C.; ¹H-NMR (DMSO-d₆) δ 12.13 (s, 1H), 11.16 (s, 1H), 10.40 (s, 1H), 7.79 (br s, 1H), 7.66 (m, 2H), 7.21 (t, J=8.8 Hz, 2H), 6.13 (s, 1H), 3.04 (q, J=7.6 Hz, 2H), 1.10 (t, J=7.6 Hz, 3H); MS (ESI) m/z 341.3 [M+1]⁺.

### Example 16

### METHYL 3-(4-CHLOROPHENYLAMINO)-5-(5-METHYL-1H-PYRAZOL-3-YLAMINO)PYRAZINE-2-CARBOXYLATE

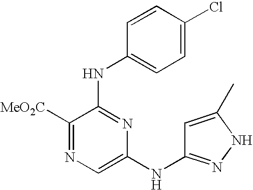

A. 3,5-Dichloropyrazine-2-carboxylic acid. To a solution of 2,2,6,6-tetramethylpiperidine (2.84 g, 20.1 mmol) in THF (250 mL) at −10° C. was added n-butyllithium (13.8 mL, 22.1 mmol). The solution was stirred for 20 minutes and cooled to −90° C. 2,6-Dichloropyrazine (3.0 g, 20.1 mmol) in THF (10 mL) was added to the solution followed by carbon dioxide (89 g, 2014 mmol) as dry ice powder. The mixture was then allowed to warm to room temperature while stirring. The reaction mixture was concentrated under reduced pressure, diluted with water (200 mL) and acidified with 10% aq. HCl solution to pH 2. Extraction with EtOAc (×3) was followed by extraction with saturated aq. NaHCO₃ solution. The aqueous solution was acidified carefully with 10% aq. HCl solution to pH 2. The solution was extracted with EtOAc (×3) and the resulting solution was washed with water and brine. The organics were dried over anhydrous MgSO4 and concentrated. The resulting solid was trituated with Hex/chloroform (1:1) and the remaining solid was filtered and washed with hexane to provide the title compound as an off-white solid (1.78 g, 9.22 mmol, 45.8% yield); ¹H NMR (400 MHz, DMSO-d₆) δ ppm 8.90 (s, 1H).

B. Methyl 3,5-dichloropyrazine-2-carboxylate. To a solution of 3,5-dichloropyrazine-2-carboxylic acid (2.68 g, 13.9 mmol) and sodium bicarbonate (1.4 g, 16.6 mmol) in DMF (20 mL) at 23° C. was added iodomethane (5.21 mL, 83 mmol). The reaction mixture was diluted with 10% aqueous citric acid solution and extracted with EtOAc. The combined organic layer was washed with water and brine, dried over MgSO₄ and concentrated under reduced pressure to give a brown solid (2.83 g, 13.6 mmol, 98% yield); ¹H NMR (400 MHz, DMSO-d₆) δ ppm 3.95 (s, 3H) 8.94 (s, 1H).

C. Methyl 5-(1-(tert-butoxycarbonyl)-5-methyl-1H-pyrazol-3-ylamino)-3-chloropyrazine-2-carboxylate. Methyl 3,5-dichloropyrazine-2-carboxylate (2.83 g, 13.7 mmol), palladium acetate (302 mg, 1.33 mmol), 4,5-bis(diphenylphosphino)-9,9-dimethylxanthene (1.63 g, 2.82 mmol), potassium carbonate (18.5 g, 135 mmol), and tert-butyl 3-amino-5-methyl-1H-pyrazole-1-carboxylate (2.70 g, 13.7 mmol) were suspended in anhydrous dioxane (95 mL). The resulting mixture was degassed with nitrogen for 5 minutes and stirred at 90° C. for one hour. The reaction was partitioned between ethyl acetate and water. The aqueous layer was extracted with ethyl acetate (3×200 mL), and the organic layers were combined and dried over magnesium sulfate. After filtration, the solvent was removed in vacuo. The residue was purified by silica gel chromatography (0-60% ethyl acetate in hexanes) to give the title compound as a yellow solid (4.20 g, 11.42 mmol, 84% yield); ¹H NMR (400 MHz, DMSO-d₆) δ ppm 1.55 (s, 9H), 2.21 (s, 3H), 3.86 (s, 3H), 6.66 (s, 1H), 8.57 (s, 1H), 10.43 (s, 1H); MS (ESI) m/z 368.3 [M+1]⁺.

D. Methyl 5-(1-(tert-butoxycarbonyl)-5-methyl-1H-pyrazol-3-ylamino)-3-(4-chlorophenylamino)pyrazine-2-carboxylate. Methyl 5-(1-(tert-butoxycarbonyl)-5-methyl-1H-pyrazol-3-ylamino)-3-chloropyrazine-2-carboxylate (4.20 g, 11.42 mmol), palladium acetate (251 mg, 1.11 mmol), 4,5-bis(diphenylphosphino)-9,9-dimethylxanthene (1.36 g, 2.35 mmol), potassium carbonate (15.4 g, 112 mmol), and 4-chloroaniline (2.185 g, 17.13 mmol) were suspended in anhydrous dioxane (80 mL). The resulting mixture was degassed with nitrogen for 5 minutes and stirred at 90° C. for one hour. The reaction was partitioned between ethyl acetate and water. The aqueous layer was extracted with ethyl acetate (3×150 mL), and the organic layers were combined and dried over magnesium sulfate. After filtration, the solvent was removed in vacuo. The residue was purified by silica gel chromatography (15-80% ethyl acetate in hexanes) to give the title compound as as a yellow solid (4.31 g, 9.39 mmol, 82% yield); 1H NMR (400 MHz, DMSO-d₆) 8 ppm 1.46-1.59 (m, 9H), 2.11 (s, 3H), 3.86 (s, 3H), 6.26 (s, 1H), 7.36-7.46 (m, 2H), 7.51 (d, J=8.98 Hz, 2H), 7.95 (s, 1H), 10.04 (d, J=9.37 Hz, 2H); MS (ESI) m/z 459.5 [M+1]⁺.

E. Methyl 3-(4-chlorophenylamino)-5-(5-methyl-1H-pyrazol-3-ylamino)pyrazine-2-carboxylate. To a solution of methyl 5-(1-(tert-butoxycarbonyl)-5-methyl-1H-pyrazol-3-ylamino)-3-(4-chlorophenylamino)pyrazine-2-carboxylate (30 mg, 0.084 mmol) in dichloromethane (5 mL) was added 4N HCl in dioxane (1 mL). The reaction was stirred at room temperature for one hour and the solvents were removed in vacuo. The crude solid was purified by reverse-phase preparative HPLC (20-80% acetonitrile+0.1% TFA in H₂O+0.1% TFA). Fractions containing product were condensed and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound as an off-white solid (10 mg, 0.029 mmol, 35% yield); m.p. 259-261° C.; 1H NMR (400 MHz, DMSO-d₆) δ ppm 2.19 (s, 3H), 3.83 (s, 3H), 6.07 (br. s., 1H), 7.39 (d, J=8.98 Hz, 2H), 7.69 (d, J=8.20 Hz, 2H), 10.17 (s, 1H), 10.23-10.38 (m, 1H), 12.13 (br. s., 1H); MS (ESI) m/z 359.1 [M+1]⁺.

### Example 17

### 3-(4-CHLOROPHENYLAMINO)—N-(2-HYDROXYETHYL)-N-METHYL-5-(5-METHYL-1H-PYRAZOL-3-YLAMINO)PYRAZINE-2-CARBOXAMIDE

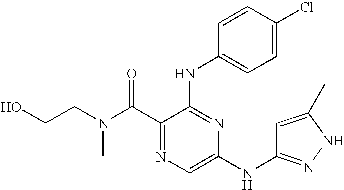

A. 3-(4-Chlorophenylamino)-5-(5-methyl-1H-pyrazol-3-ylamino)pyrazine-2-carboxylic acid. Methyl 5-(1-(tert-butoxycarbonyl)-5-methyl-1H-pyrazol-3-ylamino)-3-(4-chlorophenylamino)pyrazine-2-carboxylate (4.8 g, 10.5 mmol) was dissolved in a 1,4-dioxane/5% NaOH solution (40 mL). After stirring overnight at room temperature, the reaction mixture was acidified with 10% aq. HCl solution to pH 2. The resulting precipitate was filtered, washed with water and dried to give the title compound as an off-white solid (3.06 g, 8.88 mmol, 85% yield); ¹H NMR (400 MHz, DMSO-d₆) δ ppm 2.20 (s, 3H), 6.10 (br. s., 1H), 7.38 (d, J=8.98 Hz, 2H), 7.70 (d, J=8.59 Hz, 2H), 7.76-8.00 (m, 1H), 10.24 (br. s., 1H), 10.48 (s, 1H), 12.13 (br. s., 1H), 12.51 (br. s., 1H); MS (ESI) m/z 345.3 [M+1]⁺.

B. 3-(4-Chlorophenylamino)-N-(2-hydroxyethyl)-N-methyl-5-(5-methyl-1H-pyrazol-3-ylamino)pyrazine-2-carboxamide. 3-(4-Chlorophenylamino)-5-(5-methyl-1H-pyrazol-3-ylamino)pyrazine-2-carboxylic acid (50 mg, 0.145 mmol), 2-(methylamino)ethanol (16.3 mg, 0.218 mmol), N,N-diisopropylethylamine (0.076 mL, 0.435 mmol) and HATU (83 mg, 0.218 mmol) were dissolved in DMF (1.5 mL) and allowed to stir for 1 hour at room temperature. The reaction mixture was purified by reverse-phase preparative HPLC (10-70% acetonitrile+0.1% TFA in H₂O+0.1% TFA). Fractions containing product were condensed and neutralized on a Strata-XC ion exchange column (Phenomenex). The product was loaded and the column washed successively with water and methanol. The product was released with 5% ammonium hydroxide in methanol and product containing eluent was concentrated under reduced pressure and dried to give the title compound as an off-white solid (341 mg, 0.084 mmol, 58.3%); m.p. 191-192° C.; ¹H NMR (400 MHz, DMSO-d₆) δ ppm 2.20 (s, 3H), 3.03 (br. s., 2H), 3.18-3.29 (m, 1H), 3.48-3.59 (m, 1H), 3.63 (br. s., 3H), 4.82 (br. s., 1H), 6.05 (br. s., 1H), 7.33 (d, J=8.98 Hz, 2H), 7.65 (d, J=8.98 Hz, 2H), 7.87 (br. s., 1H), 9.85 (s, 2H), 12.01 (s, 1H); MS (ESI) m/z 402.2 [M+1]⁺.

### Example 18

### METHYL 5-(3-CYANO-6-(5-METHYL-1H-PYRAZOL-3-YLAMINO)PYRAZIN-2-YLAMINO)-2-METHYLPHENYLCARBAMATE

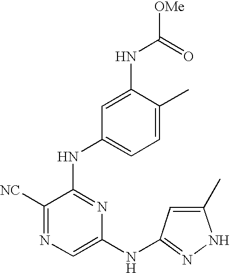

A. Methyl 5-(3-carbamoyl-6-(5-methyl-1H-pyrazol-3-ylamino)pyrazin-2-ylamino)-2-methylphenylcarbamate. To methyl 5-(1-(tert-butoxycarbonyl)-5-methyl-1H-pyrazol-3-ylamino)-3-(3-(methoxycarbonylamino)-4-methylphenylamino)pyrazine-2-carboxylate (0.811 mmol, crude material as described previously) was added a solution of ammonia in methanol (7N). The mixture was heated to 80° C. for 5 days to ensure the majority of starting material was consumed. Solvent was removed in-vacuo and the crude residue was dried under high vacuum overnight to afford a crude product which was used directly in the next reaction; MS (ESI) m/z 397.5 [M+1]⁺.

B. Methyl 5-(3-cyano-6-(5-methyl-1H-pyrazol-3-ylamino)pyrazin-2-ylamino)-2-methylphenylcarbamate. Methyl 5-(3-carbamoyl-6-(5-methyl-1H-pyrazol-3-ylamino)pyrazin-2-ylamino)-2-methylphenylcarbamate (unpurified from Step A) was dissolved in phosphorus oxychloride (5 mL, 53.6 mmol) and the mixture was heated to 90° C. for 1 h. The phosphorus oxychloride was removed and the crude residue was purified by reverse-phase preparative HPLC (10-70% acetonitrile+0.1% TFA in H₂O+0.1% TFA). Fractions containing product were condensed and neutralized on a Strata-XC ion exchange column (Phenomenex). The product was loaded and the column washed successively with water and methanol. The product was released with 5% ammonium hydroxide in methanol and product containing eluent was concentrated under reduced pressure and dried to give the title compound a solid (26 mg, 0.068 mmol, 8% yield over 3 steps); mp 268-272; ¹H NMR (400 MHz, DMSO-d₆) δ ppm 12.04 (br. s., 1H), 10.33 (br. s., 1H), 9.10 (s, 1H), 8.87 (s, 1H), 7.52 (s, 1H), 6.95-7.29 (m, 2H), 6.07 (br. s., 1H), 3.63 (s, 3H), 2.20 (s, 3H), 2.11 (s, 3H); MS (ESI) m/z 379.5 [M+1]⁺.

### Example 19

### 3-(5-AMINO-2-METHYLPHENYL)OXAZOLIDIN-2-ONE

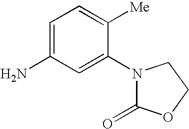

A. 3-(2-Methyl-5-nitrophenyl)oxazolidin-2-one. To 2-bromo-1-methyl-4-nitrobenzene (5 g, 23.14 mmol) and trans-1,2-diaminocyclohexane (0.278 mL, 2.314 mmol) in 1,4-dioxane (11 mL) was added oxazolidin-2-one (2.418 g, 27.8 mmol), copper (I) iodide (0.441 g, 2.314 mmol) and potassium carbonate (6.40 g, 46.3 mmol). The suspension was heated for 12 h at 110° C. The mixture was diluted with methylene chloride (200 mL), washed with saturated sodium bicarbonate (aqueous), followed by sodium chloride (aqueous, saturated). The organic layer was dried over sodium sulfate, filtered and concentrated. The crude product was purified was purified by silica gel chromatography (100% ethylacetate to 10% methanol/methylene chloride) employing silica gel chromatography (100% ethyl acetate to 10% methanol/methylene chloride) to give the title compound (2.4 g, 10.8 mmol, 47% yield).

B. 3-(5-Amino-2-methylphenyl)oxazolidin-2-one. To a solution of 3-(2-methyl-5-nitrophenyl)oxazolidin-2-one (0.444 g, 2.0 mmol) in methanol (10 mL) was added palladium on carbon (10% wt/wt) (0.100 g, 0.940 mmol). A hydrogen-filled balloon was placed over the reaction and the mixture stirred vigorously for 18 h. The reaction mixture was filtered through celite, washed with methylene chloride and concentrated to provide the title compound as a grey solid (377 mg, 98% crude yield). The crude product was used directly with no further purification.

### Example 20

### N-(5-AMINO-2-METHYLPHENYL)MORPHOLINE-4-CARBOXAMIDE

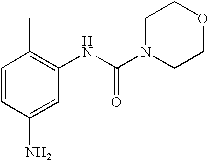

A. N-(2-methyl-5-nitrophenyl)morpholine-4-carboxamide. To a suspension of 2-isocyanato-1-methyl-4-nitrobenzene (0.891 g, 5 mmol) in THF (20 mL) was added morpholine (0.871 g, 10.00 mmol). The reaction mixture was stirred at 85° C. for 2 h., cooled to room temperature and hexane (50 mL) was added. The solid was collected to give the title compound as a white solid (1.29 g, 4.86 mmol, 97% yield); ¹H NMR (400 MHz, DMSO-d₆) δ 8.34 (s, 1H), 8.21 (d, J=2.34 Hz, 1H), 7.90 (dd, J=2.54, 8.40 Hz, 1H), 7.47 (d, J=8.20 Hz, 1H), 3.58-3.70 (m, 4H), 3.39-3.51 (m, 4H), 2.30 (s, 3H).

B. N-(5-Amino-2-methylphenyl)morpholine-4-carboxamide. To a solution of N-(2-methyl-5-nitrophenyl)morpholine-4-carboxamide (0.6 g, 2.262 mmol) in methanol (10 mL) was added palladium on carbon (0.020 g, 0.188 mmol). The reaction mixture was stirred under hydrogen (9.12 mg, 4.52 mmol) (1 atm) at 25° C. The mixture was filtered over celite and concentrated to give the title compound (0.53 g, 2.253 mmol, 100% yield); ¹H NMR (400 MHz, DMSO-d₆) δ7.79 (s, 1H), 6.79 (d, J=7.81 Hz, 1H), 6.48 (s, 1H), 6.28 (d, J=7.81 Hz, 1H), 4.79 (s, 2H), 3.59 (t, J=4.69 Hz, 4H), 3.37 (t, J=4.69 Hz, 4H), 1.98 (s, 3H).

### Example 21

### 1-(5-AMINO-2-METHYLPHENYL)-3-ISOPROPYLUREA

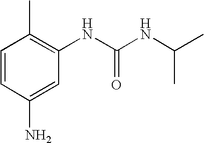

A. 1-Isopropyl-3-(2-methyl-5-nitrophenyl)urea. To a solution of 2-isocyanato-1-methyl-4-nitrobenzene (0.356 g, 2 mmol) in THF (10 mL) was added propan-2-amine (0.142 g, 2.400 mmol). The reaction mixture was stirred at 55° C. for 4 h followed by the addition of ether (20 mL). The solid was collected to afford the title compound as a white solid (0.45 g, 1.897 mmol, 95% yield); ¹H NMR (400 MHz, DMSO-d₆) δ 8.99 (s, 1H), 7.87 (s, 1H), 7.71 (d, J=8.20 Hz, 1H), 7.39 (d, J=8.20 Hz, 1H), 6.77 (d, J=7.03 Hz, 1H), 3.78 (dq, J=6.70, 13.47 Hz, 1H), 2.29 (s, 3H), 1.13 (d, J=6.25 Hz, 6H); MS (ESI) m/z 208.1 [M+1]⁺.

B. 1-(5-Amino-2-methylphenyl)-3-isopropylurea. To a solution of 1-isopropyl-3-(2-methyl-5-nitrophenyl)urea (0.45 g, 1.897 mmol) in methanol (10 mL) was added palladium on carbon (0.020 g, 0.019 mmol). The reaction mixture was stirred under hydrogen (40 psi) at 25° C. for 1 h. The reaction mixture was filtered through celite and concentrated to give the title compound (0.375 g, 1.809 mmol); ¹H NMR (400 MHz, DMSO-d₆) δ 7.22 (s, 1H), 7.18 (d, J=2.34 Hz, 1H), 6.71 (d, J=8.20 Hz, 1H), 6.36 (d, J=7.42 Hz, 1H), 6.09 (dd, J=2.34, 7.81 Hz, 1H), 4.73 (s, 2H), 3.72 (dq, J=6.61, 13.37 Hz, 1H), 1.99 (s, 3H), 1.09 (d, J=6.64 Hz, 6H); MS (ESI) m/z 208.1 [M+1]⁺.

### Example 22

### 7-AMINO-1H-BENZO[d][1,3]OXAZIN-2(4H)—ONE

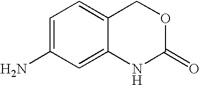

A. Methyl 2-(hydroxymethyl)-5-nitrophenylcarbamate. To a mixture of 1,4-dioxane (8 mL), water (3.00 mL), and sodium bicarbonate (aqueous, saturated) (8.00 mL) was added (2-amino-4-nitrophenyl)methanol (2.5 g, 14.87 mmol) and the suspension was cooled to 0° C. Methyl chloroformate (1.152 mL, 14.87 mmol) was added dropwise and the mixture was stirred at room temperature for 12 h. The reaction was diluted with brine (10 mL) and extracted with methylene chloride (3×, 100 mL). The organic phase was dried over sodium sulfate, filtered and concentrated to afford a crude yellow solid (3 g, 13.3 mmol, 89% yield); ¹H NMR (400 MHz, DMSO-d₆) δ ppm 9.23 (br. s., 1H), 8.48 (d, J=1.95 Hz, 1H), 7.98 (dd, J=8.20, 2.34 Hz, 1H), 7.66 (d, J=8.59 Hz, 1H), 5.70 (br. s., 1H), 4.63 (s, 2H), 3.72 (s, 3H); MS (ESI) m/z 227.3 [M+1]⁺.

B. 7-Nitro-1H-benzo[d][1,3]oxazin-2(4H)-one. To methyl 2-(hydroxymethyl)-5-nitrophenylcarbamate (0.900 g, 3.98 mmol) in toluene (10 mL) was added DBU (0.120 mL, 0.796 mmol). The suspension was heated to reflux temperature for 2 h. The reaction mixture was diluted with methylene chloride (300 mL), washed with sodium bicarbonate (aqueous, saturated) and dried with brine. After concentration in vacuo, the resulting residue was purified by silica gel chromatography (40% ethyl acetate in hexanes) to give the title compound as a solid (0.290 g, 1.49 mmol, 37% yield); ¹H NMR (400 MHz, DMSO-d₆) δ ppm 10.59 (br. s., 1H), 7.89 (dd, J=8.20, 2.34 Hz, 1H), 7.67 (d, J=2.34 Hz, 1H), 7.49 (d, J=8.59 Hz, 1H), 5.43 (s, 2H); MS (ESI) m/z 195.4 [M+1]⁺.

C. 7-Amino-1H-benzo[d][1,3]oxazin-2(4H)one. To a solution of 7-nitro-1H-benzo[d][1,3]oxazin-2(4H)-one (0.295 g, 1.519 mmol) in ethanol (10 mL) was added palladium on carbon (10% wt/wt) (0.100 g, 0.940 mmol). A hydrogen-filled balloon was placed over the reaction and the mixture stirred vigorously for 18 h. The reaction mixture was filtered through celite, washed with methylene chloride and concentrated to provide the title compound as a brown solid (0.211 g, 85% crude yield). The crude product was used directly with no further purification.

### Example 23

### 5-AMINO-2-CHLORO-N,N-DIMETHYLBENZAMIDE

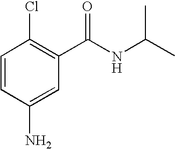

A. 5-Amino-2-chloro-N,N-dimethylbenzamide. 5-(tert-butoxycarbonylamino)-2-chlorobenzoic acid (400 mg, 1.472 mmol), HATU (672 mg, 1.767 mmol) and propan-2-amine (191 mg, 3.24 mmol) were combined in DMF (8 mL). Diisopropylethylamine (0.514 mL, 2.94 mmol) was added and the mixture stirred at room temperature for 1 h. The solvent was removed under reduced pressure and a 1:1 solution of DCM/TFA (10 mL) was added. The mixture was stirred for 2 h at room temperature and the solvent was removed under reduced pressure. The resulting residue was neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound (250 mg, 1.175 mmol, 80% yield). The product was used without additional purification; MS (ESI) m/z 213.2 [M+1]⁺.

### Example 24

### N-(5-AMINO-2-METHYLPHENYL)-2-(DIMETHYLAMINO)ACETAMIDE

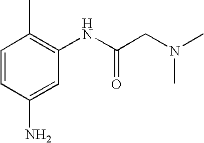

A. tert-Butyl 3-(2-(dimethylamino)acetamido)-4-Methylphenylcarbamate. To a solution of tert-butyl 3-amino-4-methylphenylcarbamate (0.445 g, 2 mmol), triethylamine (1.214 g, 12.00 mmol), in DCM (10 mL) and THF (2 mL) was added 2-(dimethylamino) acetyl chloride (0.729 g, 6.00 mmol). The reaction mixture was stirred at 25° C. for 15 h and saturated aqueous sodium chloride (25 mL) was added and extracted with ethyl acetate (3×25 mL). The organic layer was dried (MgSO₄), filtered, and concentrated. The crude product was purified by silica gel chromatography (90% ethyl acetate in hexanes) to give the title compound as light yellow solid (0.4 g, 1.301 mmol, 65.1% yield); ¹H NMR (400 MHz, DMSO-d₆) δ 9.26 (br s, 2H), 7.78 (d, J=2.34 Hz, 1H), 7.12 (dd, J=1.95, 8.20 Hz, 1H), 7.06 (d, J=8.20 Hz, 1H), 3.06 (s, 2H), 2.31 (s, 6H), 1.46 (s, 9H); MS (ESI) m/z 308.4 [M+1]⁺.

B. N-(5-Amino-2-methylphenyl)-2-(dimethylamino)acetamide. To a solution of tert-butyl 3-(2-(dimethylamino)acetamido)-4-methylphenylcarbamate (0.615 g, 2 mmol) in 1,4-dioxane (2 mL) was added 4N HCl in 1,4-dioxane (2 mL). The solution was stirred at 25° C. for 2 hours. Filtration provided the title compound as a white solid (0.30 g, 1.447 mmol, 72.4% yield); ¹H NMR (400 MHz, DMSO-d₆) δ 10.44 (s, 2H), 10.18 (br. s., 3H), 7.56 (d, J=2.34 Hz, 1H), 7.36 (d, J=8.20 Hz, 1H), 7.16 (dd, J=2.15, 8.00 Hz, 1H), 4.26 (s, 2H), 2.88 (s, 6H), 2.26 (s, 3H).

### Example 25

### N-(5-AMINO-2-METHYLPHENYL)-2-(1,3-DIOXOISOINDOLIN-2-YL)ACETAMIDE

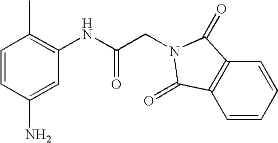

A. tert-Butyl 3-(2-(1,3-dioxoisoindolin-2-yl)acetamido)-4-methylphenylcarbamate. To a solution of tert-butyl 3-amino-4-methylphenylcarbamate (0.667 g, 3 mmol), 2-(1,3-dioxoisoindolin-2-yl)acetyl chloride (0.671 g, 3.00 mmol) in DCM (10 mL) was added diisopropylethylamine (1.048 mL, 6.00 mmol). After stirring 25° C. for 15 h, saturated aqueous sodium chloride (25 mL) was added and extracted with ethyl acetate (3×25 mL). The organic layers were combined, dried (MgSO₄), filtered, and concentrated. The crude product was purified by silica gel chromatography (100% ethyl acetate) to give the title compound as a white solid (1.15 g, 2.81 mmol, 94% yield);

¹H NMR (400 MHz, DMSO-d₆) δ 9.70 (s, 1H), 9.29 (s, 1H), 7.91-7.99 (m, 2H), 7.83-7.91 (m, 2H), 7.56 (s, 1H), 7.13 (d, J=8.20 Hz, 1H), 7.07 (d, J=8.20 Hz, 1H), 4.45 (s, 2H), 2.11 (s, 3H), 1.45 (s, 9H).

B. N-(5-Amino-2-methylphenyl)-2-(1,3-dioxoisoindolin-2-yl)acetamide.

To a solution of tert-butyl 3-(2-(1,3-dioxoisoindolin-2-yl)acetamido)-4-methylphenylcarbamate (1.15 g, 2.81 mmol) in 1,4-dioxane (10 mL) was added 4N HCl in 1,4-dioxane (5 mL, 20.00 mmol). After stirring 25° C. for 15 h, saturated aqueous sodium bicarbonate (25 mL) was added and extracted with ethyl acetate (3×25 mL). The organic layers were combined, dried (MgSO₄), filtered, and concentrated. The crude product was purified by silica gel chromatography (100% ethyl acetate) to give the title compound as a yellow solid (0.8 g, 2.59 mmol, 92% yield); ¹H NMR (400 MHz, DMSO-d₆) δ 9.47 (s, 1H), 7.91-7.98 (m, 2H), 7.84-7.91 (m, 2H), 6.82 (d, J=8.20 Hz, 1H), 6.62 (d, J=1.95 Hz, 1H), 6.32 (dd, J=2.34, 7.81 Hz, 1H), 4.89 (s, 2H), 4.42 (s, 2H), 2.02 (s, 3H); MS (ESI) m/z 310.3.4 [M+1]⁺.

### Example 26

### N′-CYCLOPENTYL-6-METHYLBENZENE-1,3-DIAMINE

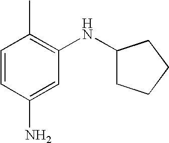

A. N¹-cyclopentyl-6-methylbenzene-1,3-diamine. To a solution of tert-butyl 3-amino-4-methylphenylcarbamate (0.43 g, 1.934 mmol) and cyclopentanone (0.65 g, 7.74 mmol) in methanol (5 mL) was added a solution of zinc(II) chloride (0.791 g, 5.80 mmol) and sodium cyanoborohydride (0.729 g, 11.61 mmol) in methanol (5 mL). The mixture was stirred 2 h and concentrated. The crude product was purified by silica gel chromatography (10% ethyl acetate in hexanes) the fractions containing product were concentrated. The Boc-intermediate was dissolved in methanol (5 mL) and 4N HCl in dioxane (5 mL) was added. The mixture was stirred 3 h and concentrated. The residue was dissolved in ethyl acetate (40 mL) followed by the addition of 4M potassium hydroxide (5 mL) and water (40 mL). The mixture was shaken and separated. The organic layer was dried (Na₂SO₄), filtered, and concentrated to afford the title compound (0.25 g, 68% yield over 2 steps); ¹H NMR (400 MHz, CHLOROFORM-d) δ 6.92 (d, J=7.81 Hz, 1H), 6.69 (s, 1H), 6.58 (d, J=7.03 Hz, 1H), 6.35 (br. s., 1H), 3.72-3.89 (m, 1H), 3.47 (br. s., 2H), 1.98-2.12 (m, 2H), 1.69-1.78 (m, 2H), 1.59-1.68 (m, 4H).

### Example 27

### 6-CHLORO-N¹-ISOPROPYLBENZENE-1,3-DIAMINE

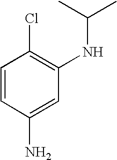

A. tert-Butyl 4-chloro-3-nitrophenylcarbamate. A solution of 4-chloro-3-nitroaniline (1.6 g, 9.27 mmol) and di-tert-butyl dicarbonate (3.24 g, 14.83 mmol) in THF (5 mL) was refluxed for 12 h. The mixture was concentrated and the residue purified by silica gel chromatography (10% ethyl acetate in hexanes) to give the title compound (7.3 mmol, 78%); ¹H NMR (400 MHz, CHLOROFORM-d) δ 8.06 (d, J=2.34 Hz, 1H), 7.36-7.49 (m, 2H), 6.67 (br. s., 1H), 1.53 (s, 9H).

B. tert-Butyl 3-amino-4-chlorophenylcarbamate. tert-Butyl 4-chloro-3-nitrophenylcarbamate (1.98 g, 7.26 mmol), iron(III) chloride hexahydrate (0.059 g, 0.218 mmol), and activated carbon (0.3 g) were combined in methanol (15 mL) and the mixture was refluxed for 10 min. Hydrazine hydrate (1.43 mL, 29.0 mmol) was added slowly and the reaction mixture was stirred at reflux for 12 h. The reaction was filtered and concentrated to ˜5 mL. The material was diluted with EtOAc and water and extracted. The organic layer was dried (Na₂SO₄), filtered, and concentrated. The residue was purified by silica gel chromatography (10% ethyl acetate in hexanes) to give the title compound (1.04 g, 59% yield); MS (ESI) m/z 243.1 [M+1]⁺.

C. tert-Butyl 4-chloro-3-(isopropylamino)phenylcarbamate. To a solution of tert-butyl 3-amino-4-chlorophenylcarbamate (0.52 g, 2.14 mmol) in acetone (2 mL) and methanol (5 mL) was added sodium cyanoborohydride (0.808 g, 12.8 mmol) and zinc(II) chloride (0.876 g, 6.43 mmol) in methanol (5 mL). After stirring for 1 day, the mixture was concentrated, dissolved in EtOAc (50 mL) and KOH (1M aq, 50 mL) was added. The mixture was extracted and the organic layer was dried (Na₂SO₄), filtered, and concentrated. The residue was purified by silica gel chromatography (20% ethyl acetate in hexanes) to give the title compound (0.36 g, 60% yield); MS (ESI) m/z 285.3 [M+1]⁺.

D. 6-Chloro-N¹-isopropylbenzene-1,3-diamine. To a solution of tert-butyl 4-chloro-3-(isopropylamino)phenylcarbamate (0.36 g) in methanol (5 mL) was added 4 N HCl in 1,4-dioxane (5 mL). The mixture was stirred 3 h and concentrated. The residue was dissolved in ethyl acetate (40 mL) followed by the addition of 4M potassium hydroxide (5 mL) and water (40 mL). The mixture was shaken and separated. The organic layer was dried (Na₂SO₄), filtered, and concentrated to afford the title compound.

### Example 28

### METHYL 5-AMINO-2-CYANOPHENYLCARBAMATE

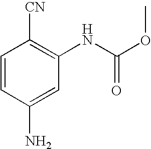

A. tert-Butyl 4-iodo-3-nitrophenylcarbamate. A solution of 4-iodo-3-nitroaniline (2.69 g, 10.19 mmol) and di-tert-butyl dicarbonate (2.89 g, 13.25 sol) in THF (10 mL) was refluxed for 1 day. The reaction mixture was concentrated pgrified by silica gel chromatography (5% ethyl acetate in hexanes) to give the title compound (3.71 g, 100% yield); ¹H NMR (400 MHz, CHLOROFORM-d) δ 8.05 (d, J=2.73 Hz, 1H), 7.88 (d, J=8.59 Hz, 1H), 7.21-7.32 (m, 1H), 6.67 (br. s., 1H), 1.52 (s, 11H)

B. tert-Butyl 3-amino-4-iodophenylcarbamate. tert-Butyl 4-iodo-3-nitrophenylcarbamate (3.71 g, 10.19 mmol), activated carbon (0.3 g) and iron(III) chloride hexahydrate (0.083 g, 0.306 mmol) were combined in methanol (10 mL) and stirred at reflux for 10 minutes. To this solution was added hydrazine hydrate (2.0 mL, 40.8 mmol) and the reaction was stirred at reflux for 1 day. The reaction mixture was concentrated and partitioned in ethyl acetate (50 mL) and water (50 mL). The mixture was extracted and the organic layer was dried (Na₂SO₄), filtered and concentrated to provide the title compound as a white solid (1.87 g, 55% yield); MS (ESI) m/z 335.1 [M+1]⁺.

C. Methyl 5-(tert-butoxycarbonylamino)-2-iodophenylcarbamate. To a solution of tert-Butyl 3-amino-4-iodophenylcarbamate (1.12 g, 3.35 mmol) and sodium hydrogencarbonate (0.845 g, 10.06 mmol) in THF (10 mL) was added methyl carbonochloridate (0.310 mL, 4.02 mmol). After stirring at 25° C. for 1 day, the reaction mixture was partitioned between ethyl acetate (50 mL) and H₂O (50 mL). The organic layer was washed with aqueous saturated NaCl (50 mL), dried (Na₂SO₄), filtered, and concentrated. The residue was purified by silica gel chromatography (25% ethyl acetate in hexanes) to give the title compound (1.1 g, 84% yield); ¹H NMR (400 MHz, CHLOROFORM-d) δ 7.99 (d, J=2.34 Hz, 1H), 7.62 (d, J=8.98 Hz, 1H), 7.13 (br. s., 1H), 6.95 (br. s., 1H), 6.53 (br. s., 1H), 3.70 (s, 3H), 1.4 (s, 9H).

D. Methyl 5-amino-2-cyanophenylcarbamate. Methyl 5-(tert-butoxycarbonylamino)-2-iodophenylcarbamate (0.202 g, 0.515 mmol) and cyanocopper (0.092 g, 1.030 mmol) were combined in NMP (0.687 mL) and heated at 90° C. for 1 day. The reaction mixture was partitioned in ethylacetate (50 mL) and NH₃—H₂O (50 mL). The organics were washed with aqueous saturated sodium chloride (50 mL), dried (Na₂SO₄), filtered, and concentrated. The residue was purified by silica gel chromatography (25% ethyl acetate in hexanes) to give the desired intermediate which was dissolved in chloroform followed by the addition of 4N HCl in 1,4-dioxane (1 mL). After stirring at 25° C. for 1 day, the mixture was concentrated, dissolved in ethyl acetate (40 mL) and KOH (3M aq, 10 mL) and water (30 mL) were added. The mixture was extracted and the organic layer was dried (Na₂SO₄), filtered, and concentrated to provide the title compound (0.1 g, 100% yield); ¹H NMR (400 MHz, CHLOROFORM-d) 68.16 (d, J=1.56 Hz, 1H), 7.46 (d, J=8.59 Hz, 1H), 7.41 (dd, J=1.76, 8.79 Hz, 1H), 7.24-7.33 (m, 1H), 7.21 (s, 1H), 7.14 (d, J=1.95 Hz, 1H), 3.81 (s, 3H).

### Example 29

### N-(5-AMINO-2-CHLOROPHENYL)ISOBUTYRAMIDE

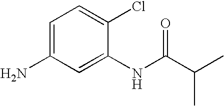

A. tert-butyl 4-chloro-3-isobutyramidophenylcarbamate. To a suspension of tert-butyl 3-amino-4-chlorophenylcarbamate (310 mg, 1.3 mmol) in dichloromethane (20 mL) and acetonitrile (5 mL) was added isobutyryl chloride (0.15 mL, 1.4 mmol) and N,N-diisopropylethylamine (0.4 mL, 2.3 mmol). The resulting mixture was stirred overnight at room temperature. The reaction was concentrated and the crude residue was purified by silica gel chromatography (10-60% ethyl acetate in hexanes) to give the title compound as an off-white solid (291 mg, 0.93 mmol, 72% yield); MS (ESI) MS (ESI) m/z 313.3 [M+1]⁺.

B. N-(5-Amino-2-chlorophenyl)isobutyramide. To a solution of tert-butyl 4-chloro-3-isobutyramidophenylcarbamate (290 mg, 0.93 mmol) in chloroform (10 mL) was added 4M HCl in dioxane (3 mL). After stirring at room temperature for 90 minutes, concentration of the reaction provided the title compound which was used directly without purification; MS (ESI) MS (ESI) m/z 213.2 [M+1]⁺.

### Example 30

### N-(5-AMINO-2-CHLOROPHENYL)-N-METHYLACETAMIDE

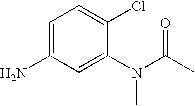

A. N-(5-amino-2-chlorophenyl)-N-methylacetamide. To a suspension of N-(5-amino-2-chlorophenyl)acetamide (500 mg, 2.7 mmol) and methyl iodide (0.2 mL, 3.2 mmol) in THF (15 mL) was added sodium hydride (60% suspension by weight: 120 mg, 3.0 mmol) in small portions. Once the addition was complete the mixture was allowed to stir for 15 minutes at room temperature before partitioning between ethyl acetate and water. The aqueous layer was washed with ethyl acetate (3×), organics were combined, dried over magnesium sulfate, filtered, and concentrated. The crude material was purified by silica gel chromatography (100% ethyl acetate) to give the title compound as a white solid (323 mg, 1.6 mmol, 59% yield); MS (ESI) MS (ESI) m/z 199.2 [M+1]⁺.

### Example 31

### 1-(5-AMINO-2-CHLOROPHENYL)PYRROLIDIN-2-ONE

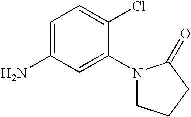

A. tert-Butyl 4-chloro-3-(4-chlorobutanamido)phenylcarbamate. To a suspension of tert-butyl 3-amino-4-chlorophenylcarbamate (1.0 g, 4.1 mmol) in dichloromethane (60 mL) and acetonitrile (10 mL) was added 4-chlorobutyryl chloride (0.5 mL, 4.5 mmol) and N,N-diisopropylethylamine (1.0 mL, 5.7 mmol). After stirring for 30 minutes at room temperature, the reaction was concentrated. The crude residue was purified by silica gel chromatography (0-40% ethyl acetate in hexanes) to give the title compound as a tan solid (990 mg, 2.9 mmol, 70% yield); MS (ESI) MS (ESI) m/z 347.4 [M+1]⁺.

B. N-(5-Amino-2-chlorophenyl)-4-chlorobutanamide. To a solution of tert-butyl 4-chloro-3-(4-chlorobutanamido)phenylcarbamate (990 mg, 2.9 mmol) in chloroform (20 mL) was added 4M HCl in dioxane (4 mL). After stirring at room temperature for 90 minutes, the reaction was concentrated. The resulting residue was neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound (532 mg, 2.2 mmol); MS (ESI) MS (ESI) m/z 247.2 [M+1]⁺.

C. 1-(5-Amino-2-chlorophenyl)pyrrolidin-2-one. To a solution of N-(5-amino-2-chlorophenyl)-4-chlorobutanamide (532 mg, 2.2 mmol) in THF (20 mL) was added sodium hydride (60% suspension by weight: 95 mg, 2.4 mmol) in small portions. Once the addition was complete the mixture was allowed to stir for 15 minutes at room temperature before partitioning between dichloromethane and water. The aqueous layer was washed with dichloromethane (3×) and the organics were combined, dried over magnesium sulfate, filtered, and concentrated. The crude residue was purified by silica gel chromatography (0-15% methanol in dichloromethane) to give the title compound as a red solid (414 mg, 2.0 mmol, 91% yield); MS (ESI) MS (ESI) m/z 211.1 [M+1]⁺.

### Example 32

### 1-(5-AMINO-2-CHLOROPHENYL)-3-PHENYLUREA

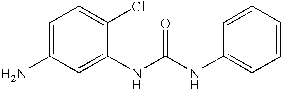

A. tert-Butyl 4-chloro-3-(3-phenylureido)phenylcarbamate. To a solution of tert-butyl 3-amino-4-chlorophenylcarbamate (358 mg, 1.5 mmol) in pyridine (3 mL) was added phenyl isocyanate (0.18 mL, 1.7 mmol). After stirring at room temperature for 2 hours, the reaction was partitioned between ethyl acetate and water. The aqueous layer was washed with ethyl acetate (3×) and the organics were combined, dried over magnesium sulfate, filtered, and concentrated The crude residue was purified by silica gel chromatography (0-50% ethyl acetate in hexanes) to give the title compound as a white solid (449 mg, 1.2 mmol, 84% yield); MS (ESI) MS (ESI) m/z 362.3 [M+1]⁺.

B. 1-(5-Amino-2-chlorophenyl)-3-phenylurea. To a solution of tert-butyl 4-chloro-3-(3-phenylureido)phenylcarbamate (449 mg, 1.2 mmol) in dichloromethane (25 mL) was added 4M HCl in dioxane (3 mL). After stirring at room temperature overnight, the reaction was concentrated and the crude solid was used directly without any further purification; MS (ESI) m/z 262.1 [M+1]⁺.

### Example 33

### 2-(3-AMINOPHENYL)-N-METHYLACETAMIDE

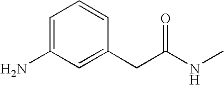

A. 2-(3-Aminophenyl)-N-methylacetamide. To a solution of 3-aminophenylacetic acid (700 mg, 3.3 mmol) in DMF (15 mL) was added N,N′-Diisopropylcarbodiimide (1.5 mL, 9.7 mmol), 1-Hydroxybenzotriazole hydrate (1.24-g, 9.2 mmol), and methyl amine (2.0 M in methanol: 4.6 mL, 9.3 mmol). After stirring at room temperature overnight, the reaction was partitioned between dichloromethane and water. The pH of the aqueous layer was adjusted to ˜12 using 5% NaOH before extracting the aqueous layer with dichloromethane (3×). The organics were combined, dried over magnesium sulfate, filtered, and concentrated. The crude residue was purified by silica gel chromatography (0-15% methanol in dichloromethane) to give the title compound as an orange oil (380 mg, 2.3 mmol, 70% yield); MS (ESI) MS (ESI) m/z 165.4 [M+1]⁺.

### Example 34

### 3-(METHOXYMETHYL)-4-METHYLANILINE

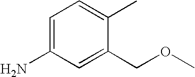

A. (5-Amino-2-methylphenyl)methanol. To a solution of methyl 5-amino-2-methylbenzoate (1 g, 6.05 mmol) in THF (10.09 mL) at 0° C. was added lithium aluminum hydride (6.05 mL, 6.05 mmol) slowly in portions. After addition was complete, the mixture was heated to 65° C. for 1 hour. The reaction was cooled to room temperature and sodium sulfate decahydrate was added and the suspension was stirred overnight. The solids were filtered, and the filtrate was concentrated to an oil. The crude residue was purified by silica gel chromatography (0-15% methanol in dichloromethane) to give the title compound as a light brown solid (588 mg, 4.3 mmol, 71% yield); MS (ESI) MS (ESI) m/z 138.4 [M+1]⁺.

B. 3-(Methoxymethyl)-4-methylaniline. To a solution of (5-amino-2-methylphenyl)methanol (386 mg, 2.8 mmol) and methyl iodide (0.194 mL, 3.1 mmol) in THF (15 mL) was added sodium hydride (124 mg, 3.1 mmol) in small portions. The mixture was allowed to stir at room temperature for 2 hours. The reaction was quenched by slow addition of brine and partitioned between ethyl acetate and water. The aqueous layer was washed with ethyl acetate (3×) and the organics were combined, dried over magnesium sulfate, filtered, and concentrated. The crude residue was purified by silica gel chromatography (0-15% methanol in dichloromethane) to give the title compound as an orange oil (285 mg, 1.9 mmol, 67% yield); MS (ESI) MS (ESI) m/z 152.3 [M+1]⁺.

### Example 35

### 2-(2-CHLORO-5-(6-CHLOROPYRAZIN-2-YLAMINO)PHENYL)PROPAN-2-OL

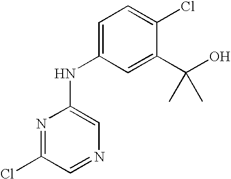

A. Methyl 5-amino-2-chlorobenzoate. To a solution of methyl 2-chloro-5-nitrobenzoate (2 g, 9.28 mmol) in ethyl acetate (40.0 mL) was added tin(II) chloride dihydrate (7.12 g, 31.5 mmol). After stirring overnight at room temperature, 1M NaOH was added slowly until pH reached ˜9. The aqueous layer was extracted with ethyl acetate (3×), the organics were combined and washed with brine, dried over magnesium sulfate, filtered, and concentrated to give methyl 5-amino-2-chlorobenzoate as a yellow oil (1.69 g, 9.11 mmol, 98% yield); MS (ESI) MS (ESI) m/z 186.1 [M+1]⁺.

B. Methyl 2-chloro-5-(6-chloropyrazin-2-ylamino)benzoate. 2,6-Dichloropyrazine (1.356 g, 9.11 mmol), methyl 5-amino-2-chlorobenzoate (1.69 g, 9.11 mmol), palladium(II) acetate (0.204 g, 0.911 mmol), xantphos (1.054 g, 1.821 mmol), potassium carbonate (10.07 g, 72.8 mmol), and dioxane (60.7 mL) were combined and degassed with nitrogen for 2 minutes before heating to 90° C. for 1 hour. The reaction was cooled to room temperature, filtered over Celite, and washed with dichloromethane. The filtrate was concentrated and purified by silica gel chromatography (10-80% ethyl acetate in hexanes) to give the title compound as a yellow solid (502 mg, 1.68 mmol, 18% yield); MS (ESI) MS (ESI) m/z 298.5 [M+1]⁺.

C. 2-(2-Chloro-5-(6-chloropyrazin-2-ylamino)phenyl)propan-2-ol. To a solution of methyl 2-chloro-5-(6-chloropyrazin-2-ylamino)benzoate (500 mg, 1.68 mmol) in THF (33.5 mL) was added methylmagnesium bromide (1.4 M) portionwise (6 mL, 8.4 mmol over 5 min). After stirring at room temperature for 30 minutes, a second portion of methylmagnesium bromide (1.4 M) (6 mL, 8.4 mmol over 5 min) was added. The reaction mixture was stirred at room temperature for an additional 30 minutes and cooled to 0° C. before slowly quenching with ˜30 mL of saturated sodium bicarbonate. The aqueous layer was extracted with ethyl acetate (3×), the organics were combined, dried over magnesium sulfate, filtered and concentrated to give the title compound as a yellow solid (500 mg, 1.68 mmol, 100% yield); ¹H-NMR (DMSO-d₆) δ 10.03 (s, 1H), 8.19 (s, 1H), 8.00 (s, 1H), 7.91 (d, J=2.73 Hz, 1H), 7.80 (dd, J=8.59, 2.73 Hz, 1H), 7.34 (d, J=8.59 Hz, 1H), 5.33 (s, 1H), 1.60 (s, 6H)

### Example 36

### 1-(7-AMINO-3,4-DIHYDROQUINOLIN-1(2H)—YL)ETHANONE

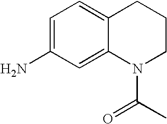

A. 1-(7-nitro-3,4-dihydroquinolin-1(2H)-yl)ethanone. To a solution of 7-nitro-1,2,3,4-tetrahydroquinoline (250 mg, 1.40 mmol) in dichloromethane (8 mL) was added acetyl chloride (0.110 mL, 1.55 mmol) and N,N-diisopropylethylamine (0.3 mL, 1.68 mmol). The mixture was stirred overnight at room temperature and concentrated. The crude material was dried under high vacuum for 2 hours before carrying the material to next step without purification (309 mg, 1.40 mmol, 100% yield).

B. 1-(7-amino-3,4-dihydroquinolin-1(2H)-yl)ethanone. To a solution of 1-(7-nitro-3,4-dihydroquinolin-1(2H)-yl)ethanone (309 mg, 1.40 mmol) in methanol (10 mL) was added palladium on carbon. The flask was evacuated and purged with hydrogen (3×), maintaining a hydrogen atmosphere with a balloon. The reaction was allowed to stir at room temperature overnight. The reaction was filtered over Celite and washed with dichloromethane. The filtrate was concentrated and dried under high vacuum for 2 hours before using directly in the next step.

### Example 37

### 2-(7-AMINO-3,4-DIHYDROISOQUINOLIN-2(1H)—YL)ETHANOL

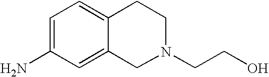

A. 2-(7-nitro-3,4-dihydroisoquinolin-2(1H)-yl)ethanol. To a suspension of 7-nitro-1,2,3,4-tetrahydroisoquinoline hydrochloride (500 mg, 2.33 mmol) and potassium carbonate (1610 mg, 11.65 mmol) in acetonitrile (155 mL) was added 2-iodoethanol (0.22 mL, 2.79 mmol). After heating at reflux overnight, the reaction was concentrated and the crude material partitioned between ethyl acetate and water. The aqueous layer was washed with ethyl acetate (3×) and the organics were combined, dried over magnesium sulfate, filtered, and concentrated. The crude residue was purified by silica gel chromatography (0-15% methanol in dichloromethane) to give the title compound as a brown oil (159 mg, 0.72 mmol, 31% yield); MS (ESI) MS (ESI) m/z 223.4 [M+1]⁺

B. 2-(7-Amino-3,4-dihydroisoquinolin-2(1H)-yl)ethanol. To a solution of 2-(7-nitro-3,4-dihydroisoquinolin-2(1H)-yl)ethanol (155 mg, 0.70 mmol) in methanol (10 mL) was added palladium on carbon. The reaction vessel was evacuated and flushed with hydrogen (3×), maintaining a hydrogen atmosphere with a balloon and stirring at room temperature overnight. The reaction was filtered over Celite and washed with dichloromethane. The filtrate was concentrated to give the title compound 1 as a light brown oil (112 mg, 0.58 mmol, 84% yield); MS (ESI) MS (ESI) m/z 193.4 [M+1]⁺

### Example 38

### N-(5-AMINO-2-METHYLPHENYL)-2-HYDROXYACETAMIDE

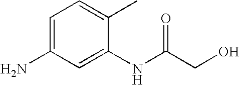

A. Methyl 2-(5-(tert-butoxycarbonylamino)-2-methylphenylamino)-2-oxoacetate. To a solution of tert-butyl 3-amino-4-methylphenylcarbamate (500 mg, 2.25 mmol) in dichloromethane (25 mL) was added methyl 2-chloro-2-oxoacetate (0.25 mL, 2.70 mmol) and N,N-diisopropylethylamine (0.6 mL, 3.4 mmol). The reaction was stirred at room temperature overnight and concentrated. The crude residue was purified by silica gel chromatography (10-70% ethyl acetate in hexanes) to give the title compound as a white solid (572 mg, 1.86 mmol, 82% yield); MS (ESI) MS (ESI) m/z 309.5 [M+1]⁺

B. N-(5-amino-2-methylphenyl)-2-hydroxyacetamide. To a solution of methyl 2-(5-(tert-butoxycarbonylamino)-2-methylphenylamino)-2-oxoacetate (409 mg, 1.33 mmol) in THF (13.3 mL) at 0° C. was slowly added lithium aluminum hydride (1.0 M: 1.5 mL, 1.5 mmol). The reaction mixture was stirred for 10 minutes and sodium sulfate decahydrate was added. After stirring at room temperature for 1 hour, the solids were filtered off and the filtrate was concentrated. The residue was acidified with 4 mL of 4 M HCl in dioxane while stirring at room temperature. After 3 hours solvents were removed and the resulting residue was neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound as a white solid (192 mg, 1.07 mmol, 80% yield); MS (ESI) MS (ESI) m/z 181.1 [M+1]⁺

### Example 39

### N-(5-AMINO-2-METHYLPHENYL)-3-HYDROXYPROPANAMIDE

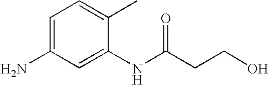

A. N-(5-amino-2-methylphenyl)-3-hydroxypropanamide. To a solution of 3-hydroxypropanoic acid (2.03 g, 6.75 mmol) in DMF (7.5 mL) was added HOBt (1.03 g, 6.75 mmol) and EDC (1.29 g, 6.75 mmol). After 5 minutes, tert-butyl 3-amino-4-methylphenylcarbamate (750 mg, 3.37 mmol) was added and the reaction was stirred at room temperature. After 2 hours the reaction was partitioned between ethyl acetate and water, washing the aqueous layer with ethyl acetate (2×). The organics were combined, dried over magnesium sulfate, filtered, and concentrated to an oil. The crude residue was purified by silica gel chromatography (2-12% methanol in dichloromethane) and the fractions containing product were combined and condensed to a colorless oil. The product was dissolved in dichloromethane (10 mL), acidified with 6 mL of 4 M HCl and stirred at room temperature for 4 hours The residue was neutralized on a Strata-XC ion exchange column (Phenomenex). The product was loaded and the column washed successively with water, and methanol. The product was released with 5% ammonium hydroxide in methanol and product containing eluent was concentrated under reduced pressure and dried to give the title compound as a colorless oil (131 mg, 0.67 mmol, 20% yield); MS (ESI) MS (ESI) m/z 195.1 [M+1]⁺

### Example 40

### 1-(5-AMINO-2-METHYLPHENYL)-3-PHENYLUREA

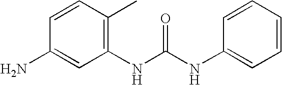

A. 1-(5-amino-2-methylphenyl)-3-phenylurea. A solution of tert-butyl 3-amino-4-methylphenylcarbamate (0.6 g, 2.70 mmol) and phenyl isocyanate (0.324 mL, 2.97 mmol) in chloroform (25 mL) was heated at 55° C. for 18 hours. The white solid was filtered to give tert-butyl 4-methyl-3-(3-phenylureido)phenylcarbamate (900 mg). This intermediate was dissolved in methylene chloride (20 mL) and TFA was added (4 mL). of very clean boc protected 1-(5-amino-2-methylphenyl)-3-phenylurea. After stirring for 1 hour, the reaction was concentrated and neutralized on a Strata-XC ion exchange column (Phenomenex). The product was loaded and the column washed successively with water, acetonitrile, and methanol. The product was released with 5% ammonium hydroxide in methanol and product containing eluent was concentrated under reduced pressure and dried to give the title compound; MS (ESI) m/z 242.2 [M+1]⁺.

### Example 41

### 5-AMINO-2-METHYLPHENYL METHYL CARBONATE

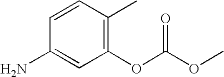

A. 5-Amino-2-methylphenyl methyl carbonate. To a solution of 2-methyl-5-nitrophenol (1.5 g, 9.80 mmol) and diisopropyldiethyl amine (2.22 mL, 12.7 mmol) in methylene chloride (50 mL) was added methyl chloroformate (0.91 mL, 11.7 mmol). The reaction was allowed to stir at room temperature for 3 hours and was washed with saturated aqueous sodium bicarbonate. The organics were dried, filtered and concentrated. The resulting methyl 2-methyl-5-nitrophenyl carbonate (1.95 g, 9.23 mmol) was dissolved in methanol (50 mL) and catalytic Pd—C was added. The reaction was stirred under a balloon of hydrogen for 20 hours, filtered through celite and concentrated to give the title compound as an oil; MS (ESI) m/z 182.1 [M+1]⁺.

### 5.2 Biological Examples

### 5.2.1 IKK-2 Inhibition Assay

IKK2-NEMO (Hexa-His tagged IKK-2 and Strep tagged NEMO) were coexpressed in a baculoviral expression system and purified by affinity column chromatography. GST-IκBα was provided by Ares-Serono S. A. The antibodies used for detection of phosphorylated GST-IκBα (Europium labeled Anti-mouse IgG, Cy5 labeled Anti-GST and mouse Anti-phospho-IκBα) were commercially available from, e.g., Amersham and Cell Signaling Technology. All other assay components were also commercially available from, e.g., Sigma-Aldrich.

Assays were run in black 384-well flat bottom plates (Costar 3710). Final assay buffer contained 50 mM HEPES buffer (pH 7.6), 10 mM MgCl₂, 1 mM EGTA, 1 mM DTT, and 0.004% Triton X-100. Test compounds were serially diluted 1:3 in 100% DMSO prior to diluting 10-fold with assay buffer to prepare a 10-point dose response range. All data points were measured in duplicate. To 5 μL of test compound dissolved in 10% DMSO, was added 10 μL of an enzyme solution containing 1.7 μg/mL of IKK2-NEMO in final assay buffer. The reaction was initiated by addition of 10 μL of Detection Mixture that contained ATP (3.75 μM), mouse Anti-phospho-IκBα (75 ng/mL), GST-IκBα (1.26 μg/mL), and Europium labeled Anti-Mouse antibody (750 ng/mL) in final assay buffer. The reaction was allowed to proceed for 45 minutes at room temperature. The reaction was stopped by addition of 10 μL of a solution containing EDTA (70 mM) and Cy5 labeled anti-GST (40 μg/mL). The plate was shaken for 20 seconds and allowed to stand, and covered in the dark for at least 3 hours. The TR-FRET signal was read on an Analyst HT (Flash lamp Ex 330 nm, Em 665 nm [int. time 200 μs, delay 50 μs, readings per well: 100] and 620 nm [int. time 1000 μs, delay 400 μs, readings per well: 50] with a BB/UV dichroic mirror) and the data analyzed as follows. The non-linear, least squares fitting program Xlfit3 Excel Add-in (IDBS) was used to fit the dose response curves to a 4-parameter logistic model with the zero percent and 100 percent enzyme activity fixed and locked (effectively reducing to a 2-parameter fit):

$$\% Act . = \frac{100 \%}{1 + {( \frac{I}{{IC}_{50}} )}^{n}}$$

where % Act. is percent enzyme activity remaining, [I] is the test compound concentration, IC₅₀ is the concentration of test compound calculated to give 50% remaining enzyme activity, and n is the Hill slope.

Compounds in Table 1 were tested in the IKK-2 Inhibition Assay and were shown to have an IC₅₀ value of <10 μM, with some compounds having an IC₅₀ of <1 μM, others an IC₅₀ of <0.1 μM and yet others an IC₅₀ of <0.01 μM.

### 5.2.2 Reporter Gene Assay

A THP-1 cell line stably transfected with a beta-lactamase reporter gene under the control of a NF-κB response element was purchased from Invitrogen and used as a cell-based assay for detecting inhibition of IKK-2 activity (THP-1 κB-bla). Cells were maintained and passaged in growth medium containing RPMI 1640 (90%), dialyzed FBS (10%), non-essential amino acids (0.1 mM), sodium pyruvate (1 mM), antibiotic (100 U/mL), and blasticidin (5 μg/mL). All media components were purchased from Invitrogen Corporation.

Cell assays were run in black 384-well plates (BD biosciences #353962). THP-1 κB-bla cells were harvested from growth medium and resuspended in assay medium (growth medium minus blasticidin) and plated 10,000 cells per well in 36 μL. Following an overnight incubation in a 5% CO₂ incubator at 37° C., serially diluted compounds (8 point dose response range) were added at 10× in 2% DMSO (0.2% final) into corresponding wells and the plate is incubated at 37° C. for 30 minutes. LPS at a final concentration of 0.1 μg/mL in assay media was added to the test and positive control wells for activation of the pathway. Plates were further incubated at 37° C./5% CO₂ for 4 hours prior to beta-lactamase substrate loading with 6× LiveBlazer FRET B/G substrate (CCF4-AM) for an additional 2 hours. The FRET emission was read on the Molecular Device Flexstation II (Excitation filter: 409/20 nm; Emission filters: 460/40 nm and 530/30 nm). The non-linear, least squares fitting program Xlfit3 Excel Add-in (IDBS) was used to fit the 8-point dose response curves based on the DMSO (unstimulated control) as 0% activity and the LPS treated control Response Ratio (RR) as 100% activity:

$$ER = \frac{Net {Em}_{460}}{Net {Em}_{530}} RR = \frac{Stimulated ER}{Mean Unstimulated ER} ,$$

wherein ER is Emission Ratio and RR is Response Ratio for each compound. The EC₅₀ was calculated as the concentration of test compound that inhibited 50% of the LPS treated control RR.

Pyrazole Pyrazine Amine Compounds as described herein were shown to have, or will be shown to have an IC₅₀ value of <10 μM, with some compounds having an IC₅₀ of <1 μM, and others an IC₅₀ of <0.1 μM.

### 5.2.3 Systemic LPS Challenge Model

Acute in vivo efficacy was determined by the ability of orally dosed compounds to inhibit LPS-induced plasma TNFα production and hepatic phospho-IκB activation. CD-1 mice (8-10 weeks old; Charles River Laboratories) were individually dosed with compound via oral gavage before LPS was administered intravenously at 0.1 mL of an 0.5 mg/kg LPS solution. At a specific time post LPS challenge, mice were anestitized via isoflurane asphyxiation and blood was collected via orbital bleed and the liver removed for protein extraction. Endpoint analysis for mouse plasma TNFα levels were done with MesoScale mouse TNFα kits and percent inhibition per dose was calculated against vehicle treated cohorts. Statistical significance was determined by using Dunnet's One Way ANOVA analysis with GraphPad Prism software.

Measurements of hepatic phospho-IκBα inhibition upon LPS challenge were made by using the PathScan phospho-IκBα S32 kit from Cell Signaling Technology

In this model, Pyrazole Pyrazine Amine Compounds as described herein were shown to have, or will be shown to have an ED₅₀ value of <100 mg/kg, with some compounds having an ED₅₀ of <10 mg/kg and others an ED₅₀ of <1 mg/kg.

### 15.2.4 Adjuvant Induced Arthritis Model

Normal Lewis (LEW/CrIBR) rats (Charles River) weighing 125-135 g on arrival are acclimated for 4-8 days after arrival. Rats are housed in regular shoebox cages, maintained on a 12 hour light/dark cycle, and fed Harlan Teklad (#7001) 4% mouse/rat diet and water ad libitum. Acclimated animals are lightly anesthetized with Isoflurane and given ground Mycobacterium Butyricum injections (5 mg/mL in mineral oil). Each animal receives 100 μL of the mixture into the tail vein. Animals to be given vehicle or test compound are dosed q.d. (24 hr intervals) beginning day 8 (anaphylactic) or day 12 (therapeutic) using a volume of 5 mL/kg for oral solutions. Footpad measurements are taken on days 0, 8, 12, 14, 18, 20, and 21. Water displacement measurements of each left hind paw is taken with a plethysmometer (Buxco) at the same point on the tibia slightly above the talus. For endpoint measurements, a last footpad measurement is taken prior to CO₂ asphyxiation. The final body weights are collected before the left hind paw is removed and fixed in 10% buffered formalin for x-ray and histological analysis. A cumulative bone score comprised of the following parameters is assigned to each paw: calcaneal erosions: 0 or 1 point; heterotopic bone formation: 0 or 1 point; demineralization: 0 to 2 points; erosions: 0 to 2 points. Percent inhibition of paw edema and bone score are calculated using the following formula:

% Inhibition=A−B/A×100
with A=(Mean Disease Control−Mean Normal) and
B=(Mean Treated−Mean Normal)

In this model, Pyrazole Pyrazine Amine Compounds as described herein were shown to have, or will be shown to have an ED₅₀ value of <100 mg/kg, with some compounds having an ED₅₀ of <10 mg/kg and others an ED₅₀ of <1 mg/kg.

### 5.2.5 Xenograft Cancer Model

Human cancer cell lines are injected into athymic nude mice. For cells maintained in vitro, tumors are generated by injecting precisely determined numbers of cells into mice. For tumors which are best propagated in vivo, tumor fragments from donor mice are implanted into small numbers of mice for maintenance, or larger numbers of mice for study initiation. A typical efficacy study design involves administering one or more drugs to tumor-bearing mice. Additionally, reference chemotherapeutic agents (positive control) and negative controls are similarly administered and maintained. Routes of administration can include subcutaneous (SC), intraperitoneal (IP), intravenous (IV), intramuscular (IM) and oral (PO). Tumor measurements and body weights are taken over the course of the study and morbidity and mortality are recorded. Necropsy, histopathology, bacteriology, parasitology, serology and PCR can also be performed to enhance understanding of disease and drug action.

Some of the typical human cancer cell lines that can be used in the above xenograft models are: the MDA MB-231, MCF7, MDA-MB-435, and T-47D cell lines for breast cancer; the KM 12, HCT-15, COLO 205, and HT29 cell lines for colon cancer; the NCI-H460 and A549 cell lines for lung cancer; the CRW22, LNCAP, PCC-3, and DU-145 cell lines for prostate cancer; the LOX-IMVI cell line for melanoma; the SK-O V-3 and A2780 cell lines for ovarian cancer; and the CAKI-I, A498, and SN12C cell lines for renal cancer.

In this model, Pyrazole Pyrazine Amine Compounds as described herein were shown to have, or will be shown to have an ED₅₀ value of <100 mg/kg, with some compounds having an ED₅₀ of <10 mg/kg and others an ED₅₀ of <1 mg/kg.

The embodiments disclosed herein are not to be limited in scope by the specific embodiments disclosed in the examples which are intended as illustrations of a few aspects of the disclosed embodiments and any embodiments that are functionally equivalent are encompassed by the present disclosure. Indeed, various modifications of the embodiments disclosed herein are in addition to those shown and described herein will become apparent to those skilled in the art and are intended to fall within the scope of the appended claims.

A number of references have been cited, the disclosures of which are incorporated herein by reference in their entirety.

What is claimed is:

1. A compound formula (I): 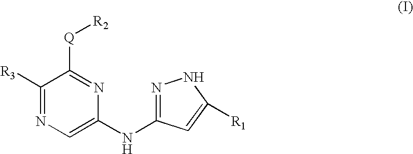
or a pharmaceutically acceptable salt, stereoisomer or tautomer thereof, wherein:
Q is a direct bond or NH;
R¹ is H or a substituted or unsubstituted (C₁₋₄)alkyl;
R² is cycloalkyl; aryl; or heterocyclyl, wherein the cycloalkyl, aryl, or heterocyclyl is optionally substituted with one or more substituted or unsubstituted C₁₋₆ alkyl; cyano; halogen; (C₁₋₆)alkoxy; aryloxy; acylamino; aminocarbonyl; urea;
(C₁₋₄)alkylsulfonylamino; NR⁴₂, C(O)OR⁵; C(O)R⁶; OC(O)R⁷; NRC(O)OR⁸; or a substituted or unsubstituted heterocyclyl;
R³ is H, CN, C(O)NR⁹R¹⁰, C(O)OR⁹, or C(O)R¹¹;
R⁴ is at each occurrence independently H, substituted or unsubstituted C₁₋₆ alkyl, or substituted or unsubstituted C₁₋₆ cycloalkyl;
R⁵, R⁶, R⁷ and R⁸ at each occurrence are independently substituted or unsubstituted C₁₋₆ alkyl, or substituted or unsubstituted C₁₋₆ cycloalkyl;
R⁹ and R¹⁰ are each independently H, substituted or unsubstituted C₁₋₄ alkyl, substituted or unsubstituted C₁₋₆ cycloalkyl, substituted or unsubstituted heterocyclyl, or substituted or unsubstituted heterocyclylalkyl; or R⁹ and R¹⁰, together with the nitrogen atom to which they are attached form a substituted or unsubstituted heterocyclyl;
R¹¹ is substituted or unsubstituted C₁₋₆ alkyl, or substituted or unsubstituted C₁₋₆ cycloalkyl; and
R is H or substituted or unsubstituted C₁₋₆ alkyl;
provided that the compound is not N-(5-methyl-1H-pyrazol-3-yl)-6-(pyridin-2-yl)pyrazin-2-amine.

2. The compound of claim 1, wherein Q is NH.

3. The compound of claim 1, wherein R¹ is methyl.

4. The compound of claim 1, wherein R² is cyclohexyl, phenyl, pyridinyl, quinolyl, isoquinolyl, tetrahydroquinolyl, tetrahydroisoquinolyl, dihydroquinolinonyl, dihydroisoquinolinonyl, benzoxazinone, dihydroindenyl, dihydroindenonyl, dihydrobenzazepinone, tetrahydrobenzazepine, benzothiazolyl, benzodioxolyl, indazolyl, benzimidazolyl, indolinyl, isoindolinyl, or indolinonyl.

5. The compound of claim 4, wherein R² is phenyl, dihydroindenyl, dihydroindenonyl, substituted with one or more substituted or unsubstituted C₁₋₆ alkyl; cyano; halogen; (C₁₋₆)alkoxy; acylamino; aminocarbonyl; urea; (C₁₋₆)alkylsulfonylamino; NR⁴₂; C(O)OR⁵; C(O)R⁶; OC(O)R⁷; or NRC(O)OR⁸.

6. The compound of claim 4, wherein R² is phenyl, dihydroindenyl, dihydroindenonyl, substituted with one or more C₁₋₄ alkyl; cyano; halogen; O(C₁₋₄alkyl); NHC(O)(C₁₋₄ alkyl); N(C₁₋₄ alkyl)C(O)(C₁₋₄ alkyl); NHC(O)(C₁₋₄ cycloalkyl); N(C₁₋₄ alkyl)C(O)(C₁₋₆ cycloalkyl); NHC(O)(heterocyclyl); N(C₁₋₄ alkyl)C(O)(heterocyclyl); C(O)NH(C₁₋₄ alkyl); C(O)N(C₁₋₄ alkyl)₂; NHC(O)NH(C₁₋₄ alkyl); N(C₁₋₄ alkyl)C(O)NH(C₁₋₄ alkyl); NHC(O)N(C₁₋₄ alkyl)₂; NHC(O)NH(C₁₋₆ cycloalkyl); N(C₁₋₄ alkyl)C(O)NH(C₁₋₆ cycloalkyl); NHC(O)NH(aryl); NHSO₂(C₁₋₄ alkyl); N(C₁₋₄ alkyl)SO₂(C₁₋₄ alkyl); NHSO₂(aryl); NH(C₁₋₄ alkyl); N(C₁₋₄ alkyl)₂; NH(C₁₋₆ cycloalkyl); N(C₁₋₄ alkyl)(C₁₋₆ cycloalkyl); C(O)O(C₁₋₄ alkyl); OC(O)(C₁₋₄ alkyl); NHC(O)O(C₁₋₄ alkyl); N(C₁₋₄ alkyl)C(O)O(C₁₋₄ alkyl); NHC(O)O(C₁₋₆ cycloalkyl); or N(C₁₋₄ alkyl)C(O)O(C₁₋₆ cycloalkyl); wherein each alkyl, cycloalkyl or heterocyclyl is substituted or unsubstituted.

7. The compound of claim 4, wherein R² is phenyl, dihydroindenyl, dihydroindenonyl, substituted with one or more Cl, F, CN, CF₃, methyl, ethyl, n-propyl, isopropyl, n-butyl, isobutyl, sec-butyl, t-butyl, O-methyl, O-ethyl, (C₁₋₄ alkyl)-OH, (CH₂)ₙC(O)OR, (CH₂)ₙC(O)NR₂, (CH₂)ₙO(C₁₋₄ alkyl), NHC(O)CH₃, NHC(O)CH₂CH₃; NHC(O)CH(CH₃)₂, NHC(O)cyclopropyl, NHC(O)cyclobutyl, NHC(O)cyclopentyl, N(CH₃)C(O)CH₃, NHC(O)(CH₂)ₙOR, NHC(O)(CH₂)ₙNR₂, NHC(O)(pyrrolidinyl), NHC(O)(morpholinyl), C(O)NHCH₃, C(O)N(CH₃)₂, NHC(O)NH(CH₃), NHC(O)NH(CH₂CH₃), NHC(O)NH(CH(CH₃)₂), NHC(O)NH(cyclopentyl), NHC(O)NH(cyclohexyl), NHC(O)NH(phenyl), NHC(O)N(CH₃)₂, NHSO₂(CH₃), NHSO₂(phenyl), NH(CH₃), N(CH₃)₂, NH(cyclopropyl), NH(cyclobutyl), NH(cyclopentyl), C(O)O(CH₃), OC(O)(CH₃), NHC(O)O(CH₃), NHC(O)O(CH₂CH₃), NHC(O)O(CH₂(CH₃)₂), NHC(O)O(cyclopropyl), NHC(O)O(cyclobutyl), NHC(O)O(cyclopentyl), N(CH₃)C(O)O(CH₃), wherein R is H or substituted or unsubstituted C₁₋₆ alkyl, and n is 1-3.

8. The compound of claim 7, wherein R² is phenyl, dihydroindenyl, dihydroindenonyl, substituted with at least one methyl, Cl or F.

9. The compound of claim 8, wherein R² is phenyl, dihydroindenyl, dihydroindenonyl, further substituted with NHC(O)CH₃, NHC(O)CH₂CH₃; NHC(O)CH(CH₃)₂, NHC(O)cyclopropyl, NHC(O)cyclobutyl, NHC(O)cyclopentyl, N(CH₃)C(O)CH₃, NHC(O)(CH₂)ₙOR, NHC(O)(CH₂)ₙNR₂, NHC(O)(pyrrolidinyl), NHC(O)(morpholinyl), NHC(O)NH(CH₃), NHC(O)NH(CH₂CH₃), NHC(O)NH(CH(CH₃)₂), NHC(O)NH(cyclopentyl), NHC(O)NH(cyclohexyl), NHC(O)NH(phenyl), NHC(O)N(CH₃)₂, NHC(O)O(CH₃), NHC(O)O(CH₂CH₃), NHC(O)O(CH₂(CH₃)₂), NHC(O)O(cyclopropyl), NHC(O)O(cyclobutyl), NHC(O)O(cyclopentyl), or N(CH₃)C(O)O(CH₃).

10. The compound of claim 9, wherein Q is NH.

11. The compound of claim 10, wherein R³ is H.

12. The compound of claim 4, wherein R² is phenyl, dihydroindenyl, dihydroisoindenonyl, substituted with a substituted or unsubstituted heterocyclyl.

13. The compound of claim 12, wherein the heterocyclyl is pyrrolidinyl, pyrrolidinonyl, oxazolidinonyl, or piperidonyl.

14. The compound from claim 13, wherein the heterocyclyl is

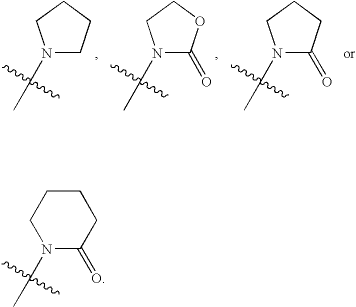

15. The compound of claim 12, wherein R² is phenyl, dihydroindenyl, dihydroindenonyl, substituted with at least one methyl, Cl or F.

16. The compound of claim 14, wherein Q is NH.

17. The compound of claim 16, wherein R³ is H.

18. The compound of claim 4, wherein R² is a heterocyclyl, optionally substituted with substituted or unsubstituted C₁₋₆ alkyl; (C₁₋₆)alkoxy; aminocarbonyl; C(O)OR⁵; or C(O)R⁶.

19. The compound of claim 18, wherein R² is a bicyclic heterocyclyl.

20. The compound of claim 18, wherein R² is pyridinyl, quinolyl, isoquinolyl, tetrahydroquinolyl, tetrahydroisoquinolyl, dihydroquinolinonyl, dihydroisoquinolinonyl, benzoxazinone, dihydroindenyl, dihydroindenonyl, dihydrobenzazepinone, tetrahydrobenzazepine, benzothiazolyl, benzodioxolyl, indazolyl, benzimidazolyl, indolinyl, isoindolinyl, or indolinonyl.

21. The compound of claim 18, wherein the heterocyclyl is substituted with methyl, ethyl, n-propyl, isopropyl, O(C₁₋₃ alkyl), (CH₂)ₘOR, (CH₂)ₘOC(O)(C₁₋₆ cycloalkyl), C(O)NH(C₁₋₄ alkyl), C(O)N(C₁₋₄ alkyl)₂, C(O)O(C₁₋₆ alkyl), C(O)(C₁₋₄ alkyl), or C(O)(C₁₋₆ cycloalkyl), wherein R is H or substituted or unsubstituted C₁₋₆ alkyl, and m is 1-3.

22. The compound of claim 21, wherein the heterocyclyl is substituted with methyl, ethyl, n-propyl, isopropyl, O— methyl, O-ethyl, (CH₂)OH, (CH₂)OC(O)CH₃, C(O)NH(CH₃), C(O)N(CH₃)₂, C(O)OCH₃, C(O)CH₃, C(O)(cyclopropyl), C(O)(cyclobutyl), or C(O)(cyclopentyl).

23. The compound of claim 18, wherein R² is 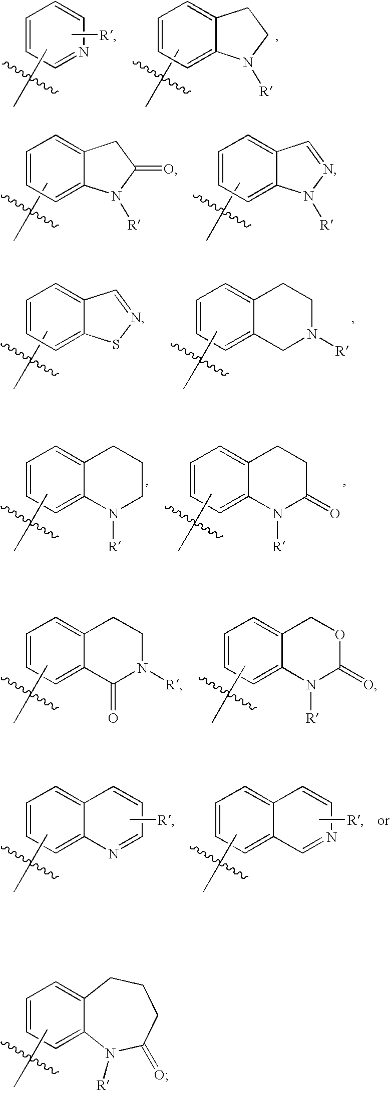
wherein R′ is H, methyl, ethyl, n-propyl, isopropyl, O(C₁₋₃ alkyl), (CH₂)ₘOR, (CH₂)ₘOC(O)(C₁₋₆ cycloalkyl), C(O)NH(C₁₋₄ alkyl), C(O)N(C₁₋₄ alkyl)₂, C(O)O(C₁₋₆ alkyl), C(O)(C₁₋₄ alkyl), or C(O)(C₁₋₆ cycloalkyl), wherein R is H or substituted or unsubstituted C₁₋₆ alkyl, and m is 1-3.

24. The compound of claim 23, wherein Q is NH.

25. The compound of claim 24, wherein R³ is H.

26. The compound of claim 1, wherein R³ is CN, C(O)NR⁹R¹⁰, or C(O)R¹¹.

27. The compound of claim 26, wherein R⁹ and R¹⁰ are each independently H, substituted or unsubstituted C₁₋₆ alkyl, substituted or unsubstituted C₁₋₆ cycloalkyl, substituted or unsubstituted heterocyclyl, or substituted or unsubstituted heterocyclylalkyl.

28. The compound of claim 27, wherein R⁹ and R¹⁰ are each independently H, methyl, ethyl, n-propyl, isopropyl, (CH₂)ₚNR₂, (CH₂)ₚNRC(O)R, (CH₂)ₚCONR₂, (CH₂)ₚOR; substituted or unsubstituted cyclopropyl, cyclobutyl, cyclopentyl, or cyclohexyl; substituted or unsubstituted azetidinyl, pyrrolidinyl, pyrrolidinonyl, piperidinyl or piperazinyl; or substituted or unsubstituted (CH₂)ₚ(azetidinyl), (CH₂)ₚ(pyrrolidinyl), (CH₂)ₚ(pyrrolidinonyl), (CH₂)ₚ(piperidinyl), or (CH₂), (piperazinyl); wherein each R is independently H or substituted or unsubstituted C₁₋₄ alkyl, and p is 1-3.

29. The compound of claim 26, wherein R⁹ and R¹⁰ together with the nitrogen atom to which they are attached form a substituted or unsubstituted heterocyclyl.

30. The compound of claim 29, wherein the heterocyclyl is pyrrolidinyl, pyrrolidinonyl, piperidinyl, piperidonyl, piperazinyl, or piperazinonyl.

31. The compound of claim 26, wherein R¹¹ is substituted or unsubstituted C₁₋₄ alkyl.

32. The compound of claim 26, wherein R³ is 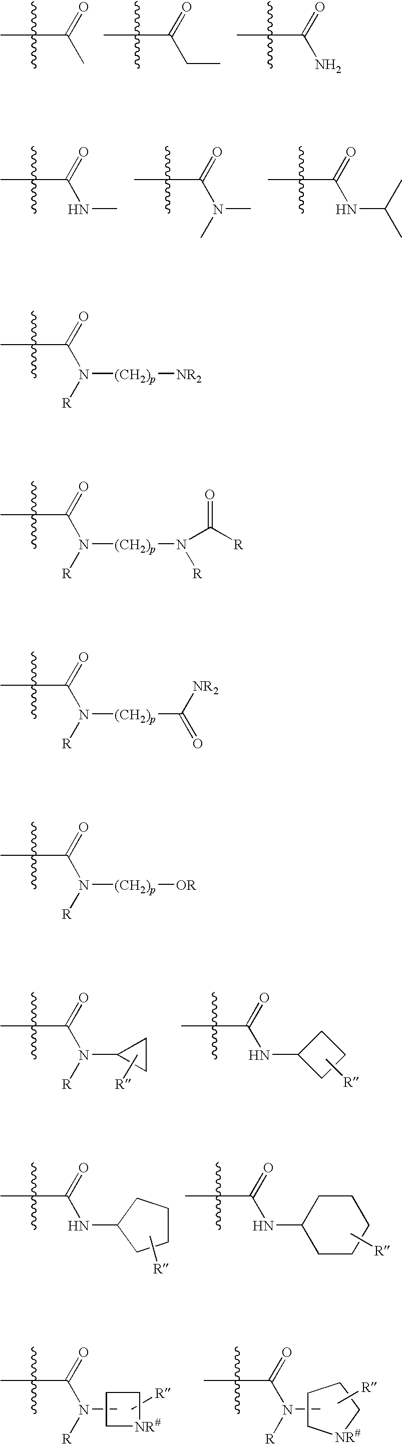 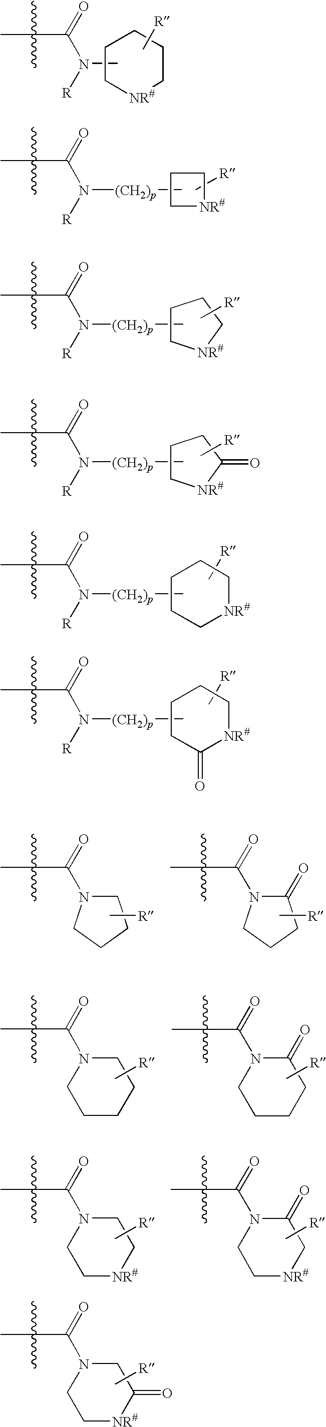
wherein each R is independently H or substituted or unsubstituted C₁₋₆ alkyl, R″ is H, C(O)C₁₋₄ alkyl), wherein the alkyl is optionally substituted or unsubstituted, R^{#} is H, NR₂, OR, (CH₂)ₚNR₂, (CH₂)ₚOR, or NR(CO)(C₁₋₄ alkyl), and p is 1-3.

33. The compound of claim 32, wherein Q is NH.

34. A compound or a pharmaceutically acceptable salt thereof, wherein the compound is:
N²-(5-Methyl-1H-pyrazol-3-yl)-N⁶-phenylpyrazine-2,6-diamine;
N²-(5-methyl-1H-pyrazol-3-yl)-N⁶-(tetrahydro-2H-pyran-4-yl)pyrazine-2,6-diamine;
N²-(2,3-dihydro-1H-inden-2-yl)-N⁶-(5-methyl-1H-pyrazol-3-yl)pyrazine-2,6-diamine;
3-(6-(5-methyl-1H-pyrazol-3-ylamino)pyrazin-2-ylamino)benzonitrile;
4-(6-(5-methyl-1H-pyrazol-3-ylamino)pyrazin-2-ylamino)benzonitrile;
6-(5-methyl-1H-pyrazol-3-yl)-N-(4-phenoxyphenyl)pyrazin-2-amine;
6-(5-methyl-1H-pyrazol-3-yl)-N-(3-phenoxyphenyl)pyrazin-2-amine;
N²-cyclohexyl-N⁶-(5-methyl-1H-pyrazol-3-yl)pyrazine-2,6-diamine;
N²-(4-chlorophenyl)-N⁶-(5-methyl-1H-pyrazol-3-yl)pyrazine-2,6-diamine;
N²-(2,3-dihydro-1H-inden-5-yl)-N⁶-(5-methyl-1H-pyrazol-3-yl)pyrazine-2,6-diamine;
N²-(5-methyl-1H-pyrazol-3-yl)-N-6-m-tolylpyrazine-2,6-diamine;
N²-(3-methoxyphenyl)-N⁶-(5-methyl-1H-pyrazol-3-yl)pyrazine-2,6-diamine;
N²-(4-chloro-3-fluorophenyl)-N⁶-(5-methyl-1H-pyrazol-3-yl)pyrazine-2,6-diamine;
N²-(4-chloro-2-fluorophenyl)-N⁶-(5-methyl-1H-pyrazol-3-yl)pyrazine-2,6-diamine;
j (2-chloro-5-(6-(5-methyl-1H-pyrazol-3-ylamino)pyrazin-2-ylamino)phenyl)methanol;
N-(2-chloro-5-(6-(5-methyl-1H-pyrazol-3-ylamino)pyrazin-2-ylamino)phenyl)acetamide;
N²-(2,4-dichlorophenyl)-N⁶-(5-methyl-1H-pyrazol-3-yl)pyrazine-2,6-diamine;
2-chloro-5-(6-(5-methyl-1H-pyrazol-3-ylamino)pyrazin-2-ylamino)phenol;
N²-(isoquinolin-6-yl)-N⁶-(5-methyl-1H-pyrazol-3-yl)pyrazine-2,6-diamine;
N²-(5-methyl-1H-pyrazol-3-yl)-N⁶-(pyridin-3-yl)pyrazine-2,6-diamine;
6-(6-(5-methyl-1H-pyrazol-3-ylamino)pyrazin-2-ylamino)-3,4-dihydronaphthalen-1(2H)-one;
N²-(5-methyl-1H-pyrazol-3-yl)-N⁶-(quinolin-6-yl)pyrazine-2,6-diamine;
N²-(5-methyl-1H-pyrazol-3-yl)-N⁶-(1,2,3,4-tetrahydroisoquinolin-6-yl)pyrazine-2,6-diamine;
N²-(5-methyl-1H-pyrazol-3-yl)-N⁶-(pyridin-2-yl)pyrazine-2,6-diamine;
N²-(5-methyl-1H-pyrazol-3-yl)-N⁶-(pyridin-4-yl)pyrazine-2,6-diamine;
N², N⁶-bis(5-methyl-1H-pyrazol-3-yl)pyrazine-2,6-diamine;
(3-(6-(5-methyl-1H-pyrazol-3-ylamino)pyrazin-2-ylamino)phenyl)methanol;
N²-(isoquinolin-7-yl)-N⁶-(5-methyl-1H-pyrazol-3-yl)pyrazine-2,6-diamine;
1-(6-(6-(5-methyl-1H-pyrazol-3-ylamino)pyrazin-2-ylamino)indolin-1-yl)ethanone;
N²-(indolin-6-yl)-N⁶-(5-methyl-1H-pyrazol-3-yl)pyrazine-2,6-diamine;
N-(3-(6-(5-methyl-1H-pyrazol-3-ylamino)pyrazin-2-ylamino)phenyl)benzamide;
N-(3-(6-(5-methyl-1H-pyrazol-3-ylamino)pyrazin-2-ylamino)phenyl)acetamide;
1-(3-(4-fluorophenylamino)-5-(5-methyl-1H-pyrazol-3-ylamino)pyrazin-2-yl)propan-1-one;
Methyl 3-(4-chlorophenylamino)-5-(5-methyl-1H-pyrazol-3-ylamino)pyrazine-2-carboxylate;
Methyl 3-(4-fluorophenylamino)-5-(5-methyl-1H-pyrazol-3-ylamino)pyrazine-2-carboxylate;
3-(4-chlorophenylamino)-N-(2-hydroxyethyl)-N-methyl-5-(5-methyl-1H-pyrazol-3-ylamino)pyrazine-2-carboxamide;
(3-(4-chlorophenylamino)-5-(5-methyl-1H-pyrazol-3-ylamino)pyrazin-2-yl)(4-hydroxypiperidin-1-yl)methanone;
N-(2-methyl-5-(6-(5-methyl-1H-pyrazol-3-ylamino)pyrazin-2-ylamino)phenyl)-2-(methylamino)acetamide;
N²-(4-chloro-3-(trifluoromethyl)phenyl)-N⁶-(5-methyl-1H-pyrazol-3-yl)pyrazine-2,6-diamine;
2-chloro-5-(6-(5-methyl-1H-pyrazol-3-ylamino)pyrazin-2-ylamino)benzonitrile;
N²-(4-chloro-3-methylphenyl)-N⁶-(5-methyl-1H-pyrazol-3-yl)pyrazine-2,6-diamine;
N²-(4-chloro-2-methylphenyl)-N⁶-(5-methyl-1H-pyrazol-3-yl)pyrazine-2,6-diamine;
N²-(4-chloro-3-methoxyphenyl)-N⁶-(5-methyl-1H-pyrazol-3-yl)pyrazine-2,6-diamine;
N²-(4-chloro-2-methoxyphenyl)-N⁶-(5-methyl-1H-pyrazol-3-yl)pyrazine-2,6-diamine;
Methyl 2-chloro-5-(6-(5-methyl-1H-pyrazol-3-ylamino)pyrazin-2-ylamino)benzoate;
N²-(3-(benzyloxy)-4-chlorophenyl)-N⁶-(5-methyl-1H-pyrazol-3-yl)pyrazine-2,6-diamine;
N²-(5-methyl-1H-pyrazol-3-yl)-N⁶-(3-(phenoxymethyl)phenyl)pyrazine-2,6-diamine;
N²-(4-chloro-3-(methoxymethyl)phenyl)-N⁶-(5-methyl-1H-pyrazol-3-yl)pyrazine-2,6-diamine;
1-(3-(6-(5-methyl-1H-pyrazol-3-ylamino)pyrazin-2-ylamino)phenyl)pyrrolidin-2-one;
N-(2-fluoro-5-(6-(5-methyl-1H-pyrazol-3-ylamino)pyrazin-2-ylamino)phenyl)acetamide;
N-(2-fluoro-5-(6-(5-methyl-1H-pyrazol-3-ylamino)pyrazin-2-ylamino)phenyl)methanesulfonamide;
2-(2-chloro-5-(6-(5-methyl-1H-pyrazol-3-ylamino)pyrazin-2-ylamino)phenyl)propan-2-ol;
2-(2-methyl-5-(6-(5-methyl-1H-pyrazol-3-ylamino)pyrazin-2-ylamino)phenyl)propan-2-ol;
N²-(3-tert-butylphenyl)-N⁶-(5-methyl-1H-pyrazol-3-yl)pyrazine-2,6-diamine;
2-chloro-N-isopropyl-5-(6-(5-methyl-1H-pyrazol-3-ylamino)pyrazin-2-ylamino)benzamide;
N²-(4-fluoro-3-methoxyphenyl)-N⁶-(5-methyl-1H-pyrazol-3-yl)pyrazine-2,6-diamine;
2-(dimethylamino)-N-(2-methyl-5-(6-(5-methyl-1H-pyrazol-3-ylamino)pyrazin-2-ylamino)phenyl)acetamide;
2-amino-N-(2-methyl-5-(6-(5-methyl-1H-pyrazol-3-ylamino)pyrazin-2-ylamino)phenyl)acetamide;
Methyl 2-isopropyl-5-(6-(5-methyl-1H-pyrazol-3-ylamino)pyrazin-2-ylamino)phenylcarbamate;
Methyl 5-(6-(5-methyl-1H-pyrazol-3-ylamino)pyrazin-2-ylamino)-2-propylphenylcarbamate;
N-(2-methyl-5-(6-(5-methyl-1H-pyrazol-3-ylamino)pyrazin-2-ylamino)phenyl)morpholine-4-carboxamide;
1-isopropyl-3-(2-methyl-5-(6-(5-methyl-1H-pyrazol-3-ylamino)pyrazin-2-ylamino)phenyl)urea;
N²-(3-(cyclopentylamino)-4-methylphenyl)-N⁶-(5-methyl-1H-pyrazol-3-yl)pyrazine-2,6-diamine;
N²-(3-(isopropylamino)-4-methylphenyl)-N⁶-(5-methyl-1H-pyrazol-3-yl)pyrazine-2,6-diamine;
N²-(4-chloro-3-(isopropylamino)phenyl)-N⁶-(5-methyl-1H-pyrazol-3-yl)pyrazine-2,6-diamine;
N²-(4-chloro-3-(cyclopentylamino)phenyl)-N⁶-(5-methyl-1H-pyrazol-3-yl)pyrazine-2,6-diamine;
N²-(4-fluoro-3-(isopropylamino)phenyl)-N⁶-(5-methyl-1H-pyrazol-3-yl)pyrazine-2,6-diamine;
Methyl 2-cyano-5-(6-(5-methyl-1H-pyrazol-3-ylamino)pyrazin-2-ylamino)phenylcarbamate;
2-(cyclopentylamino)-4-(6-(5-methyl-1H-pyrazol-3-ylamino)pyrazin-2-ylamino)benzonitrile;
8-(6-(5-methyl-1H-pyrazol-3-ylamino)pyrazin-2-ylamino)octahydro-1H-benzo[b]azepin-2(3H)-one;
N²-(5-methyl-1H-pyrazol-3-yl)-N⁶-(2,3,4,5-tetrahydro-1H-benzo[b]azepin-8-yl)pyrazine-2,6-diamine;
Ethyl 2-methyl-5-(6-(5-methyl-1H-pyrazol-3-ylamino)pyrazin-2-ylamino)phenylcarbamate;
Isopropyl 2-methyl-5-(6-(5-methyl-1H-pyrazol-3-ylamino)pyrazin-2-ylamino)phenylcarbamate;
1-(2-methyl-5-(6-(5-methyl-1H-pyrazol-3-ylamino)pyrazin-2-ylamino)phenyl)-3-phenylurea;
Methyl 2-methyl-5-(6-(5-methyl-1H-pyrazol-3-ylamino)pyrazin-2-ylamino)phenyl carbonate;
Methyl 2-(2-methyl-5-(6-(5-methyl-1H-pyrazol-3-ylamino)pyrazin-2-ylamino)phenyl)acetate;
Methyl methyl(2-methyl-5-(6-(5-methyl-1H-pyrazol-3-ylamino)pyrazin-2-ylamino)phenyl)carbamate;
1,1-dimethyl-3-(2-methyl-5-(6-(5-methyl-1H-pyrazol-3-ylamino)pyrazin-2-ylamino)phenyl)urea;
2-(2-methyl-5-(6-(5-methyl-1H-pyrazol-3-ylamino)pyrazin-2-ylamino)phenyl)acetamide;
N²-(5-methyl-1H-pyrazol-3-yl)-N⁶-(4-methyl-3-(pyrrolidin-1-yl)phenyl)pyrazine-2,6-diamine;
1,3-dimethyl-1-(2-methyl-5-(6-(5-methyl-1H-pyrazol-3-ylamino)pyrazin-2-ylamino)phenyl)urea;
N²-(4-chloro-3-(pyrrolidin-1-yl)phenyl)-N⁶-(5-methyl-1H-pyrazol-3-yl)pyrazine-2,6-diamine;
1-(2-methyl-5-(6-(5-methyl-1H-pyrazol-3-ylamino)pyrazin-2-ylamino)phenyl)ethanol;
6-(6-(5-methyl-1H-pyrazol-3-ylamino)pyrazin-2-ylamino)-2,3-dihydro-1H-inden-1-ol;
N-(2-methyl-5-(6-(5-methyl-1H-pyrazol-3-ylamino)pyrazin-2-ylamino)phenyl)cyclopentanecarboxamide;
Cyclobutyl 2-methyl-5-(6-(5-methyl-1H-pyrazol-3-ylamino)pyrazin-2-ylamino)phenylcarbamate;
1-(2-chloro-5-(6-(5-methyl-1H-pyrazol-3-ylamino)pyrazin-2-ylamino)phenyl)ethanol;
N²-(6-methoxypyridin-3-yl)-N⁶-(5-methyl-1H-pyrazol-3-yl)pyrazine-2,6-diamine;
(5-(6-(5-methyl-1H-pyrazol-3-ylamino)pyrazin-2-ylamino)-2-(trifluoromethyl)phenyl)methanol;
N²-(3-(methoxymethyl)-4-(trifluoromethyl)phenyl)-N⁶-(5-methyl-1H-pyrazol-3-yl)pyrazine-2,6-diamine;
Methyl 2-methyl-5-(6-(5-methyl-1H-pyrazol-3-ylamino)pyrazin-2-ylamino)phenylcarbamate;
N²-(3,4-dimethylphenyl)-N⁶-(5-methyl-1H-pyrazol-3-yl)pyrazine-2,6-diamine;
N²-(3-methoxy-4-methylphenyl)-N⁶-(5-methyl-1H-pyrazol-3-yl)pyrazine-2,6-diamine;
N²-(3-fluoro-4-methylphenyl)-N⁶-(5-methyl-1H-pyrazol-3-yl)pyrazine-2,6-diamine;
N²-(4-ethylphenyl)-N⁶-(5-methyl-1H-pyrazol-3-yl)pyrazine-2,6-diamine;
Methyl 2-methoxy-5-(6-(5-methyl-1H-pyrazol-3-ylamino)pyrazin-2-ylamino)phenylcarbamate;
N-(2-methyl-5-(6-(5-methyl-1H-pyrazol-3-ylamino)pyrazin-2-ylamino)phenyl)pyrrolidine-1-carboxamide;
4-methyl-N-(2-methyl-5-(6-(5-methyl-1H-pyrazol-3-ylamino)pyrazin-2-ylamino)phenyl)piperazine-1-carboxamide;
Methyl 2,3-dimethyl-5-(6-(5-methyl-1H-pyrazol-3-ylamino)pyrazin-2-ylamino)phenylcarbamate;
(R)—N-(2-methyl-5-(6-(5-methyl-1H-pyrazol-3-ylamino)pyrazin-2-ylamino)phenyl)pyrrolidine-2-carboxamide;
(S)—N-(2-methyl-5-(6-(5-methyl-1H-pyrazol-3-ylamino)pyrazin-2-ylamino)phenyl)pyrrolidine-2-carboxamide;
N²-(5-methyl-1H-pyrazol-3-yl)-N⁶-(1,2,3,4-tetrahydroquinolin-7-yl)pyrazine-2,6-diamine;
N²-(5-methyl-1H-pyrazol-3-yl)-N⁶-(1,2,3,4-tetrahydroisoquinolin-7-yl)pyrazine-2,6-diamine;
N-(2-ethyl-5-(6-(5-methyl-1H-pyrazol-3-ylamino)pyrazin-2-ylamino)phenyl)acetamide;
Methyl 2-ethyl-5-(6-(5-methyl-1H-pyrazol-3-ylamino)pyrazin-2-ylamino)phenylcarbamate;
Ethyl 2-ethyl-5-(6-(5-methyl-1H-pyrazol-3-ylamino)pyrazin-2-ylamino)phenylcarbamate;
N-(2-methyl-5-(6-(5-methyl-1H-pyrazol-3-ylamino)pyrazin-2-ylamino)phenyl)cyclopropanecarboxamide;
1-(7-(6-(5-methyl-1H-pyrazol-3-ylamino)pyrazin-2-ylamino)-3,4-dihydroisoquinolin-2(1H)-yl)ethanone;
N²-(5-methyl-1H-pyrazol-3-yl)-N⁶-(6-methylpyridin-3-yl)pyrazine-2,6-diamine;
3-(2-methyl-5-(6-(5-methyl-1H-pyrazol-3-ylamino)pyrazin-2-ylamino)phenyl)oxazolidin-2-one;
N²-(2-methyl-1,2,3,4-tetrahydroisoquinolin-7-yl)-N⁶-(5-methyl-1H-pyrazol-3-yl)pyrazine-2,6-diamine;
7-(6-(5-methyl-1H-pyrazol-3-ylamino)pyrazin-2-ylamino)-1H-benzo[d][1,3]oxazin-2(4H)-one;
N²-(2-methyl-1,2,3,4-tetrahydroisoquinolin-7-yl)-N⁶-(5-methyl-1H-pyrazol-3-yl)pyrazine-2,6-diamine;
1-(2-methyl-5-(6-(5-methyl-1H-pyrazol-3-ylamino)pyrazin-2-ylamino)phenyl)imidazolidin-2-one;
N²-(5-methyl-1H-pyrazol-3-yl)-N⁶-(6-methylpyridin-3-yl)pyrazine-2,6-diamine;
N²-(4-fluorophenyl)-N⁶-(5-methyl-1H-pyrazol-3-yl)pyrazine-2,6-diamine;
N²-(5-methyl-1H-pyrazol-3-yl)-N⁶-(4-(trifluoromethyl)phenyl)pyrazine-2,6-diamine;
N²-(3,4-dichlorophenyl)-N⁶-(5-methyl-1H-pyrazol-3-yl)pyrazine-2,6-diamine;
N²-(2-fluorophenyl)-N⁶-(5-methyl-1H-pyrazol-3-yl)pyrazine-2,6-diamine;
N-(2-chloro-5-(6-(5-methyl-1H-pyrazol-3-ylamino)pyrazin-2-ylamino)phenyl)isobutyramide;
N-(2-chloro-5-(6-(5-methyl-1H-pyrazol-3-ylamino)pyrazin-2-ylamino)phenyl)cyclohexanecarboxamide;
N-(2-chloro-5-(6-(5-methyl-1H-pyrazol-3-ylamino)pyrazin-2-ylamino)phenyl)-N-methylacetamide;
1-(2-chloro-5-(6-(5-methyl-1H-pyrazol-3-ylamino)pyrazin-2-ylamino)phenyl)pyrrolidin-2-one;
1-(2-chloro-5-(6-(5-methyl-1H-pyrazol-3-ylamino)pyrazin-2-ylamino)phenyl)-3-phenylurea;
1-(2-chloro-5-(6-(5-methyl-1H-pyrazol-3-ylamino)pyrazin-2-ylamino)phenyl)-3-methylurea;
Methyl 2-chloro-5-(6-(5-methyl-1H-pyrazol-3-ylamino)pyrazin-2-ylamino)phenylcarbamate;
1-(2-fluoro-5-(6-(5-methyl-1H-pyrazol-3-ylamino)pyrazin-2-ylamino)phenyl)pyrrolidin-2-one;
1-(2-fluoro-5-(6-(5-methyl-1H-pyrazol-3-ylamino)pyrazin-2-ylamino)phenyl)-3-methylurea;
1-(2-fluoro-5-(6-(5-methyl-1H-pyrazol-3-ylamino)pyrazin-2-ylamino)phenyl)-3-phenylurea;
methyl 2-fluoro-5-(6-(5-methyl-1H-pyrazol-3-ylamino)pyrazin-2-ylamino)phenylcarbamate;
N-(2-fluoro-5-(6-(5-methyl-1H-pyrazol-3-ylamino)pyrazin-2-ylamino)phenyl)-2-phenylacetamide;
N-(2-chloro-5-(6-(5-methyl-1H-pyrazol-3-ylamino)pyrazin-2-ylamino)phenyl)-2-phenylacetamide;
1-(2-chloro-5-(6-(5-methyl-1H-pyrazol-3-ylamino)pyrazin-2-ylamino)phenyl)-3-cyclohexylurea;
1-cyclohexyl-3-(2-fluoro-5-(6-(5-methyl-1H-pyrazol-3-ylamino)pyrazin-2-ylamino)phenyl)urea;
N-methyl-2-(3-(6-(5-methyl-1H-pyrazol-3-ylamino)pyrazin-2-ylamino)phenyl)acetamide;
N-(2-methyl-5-(6-(5-methyl-1H-pyrazol-3-ylamino)pyrazin-2-ylamino)phenyl)acetamide;
1-methyl-3-(2-methyl-5-(6-(5-methyl-1H-pyrazol-3-ylamino)pyrazin-2-ylamino)phenyl)urea;
N²-(benzo[d]thiazol-5-yl)-N⁶-(5-methyl-1H-pyrazol-3-yl)pyrazine-2,6-diamine;
N²-(benzo[d][1,3]dioxol-5-yl)-N⁶-(5-methyl-1H-pyrazol-3-yl)pyrazine-2,6-diamine;
N-(2-methyl-5-(6-(5-methyl-1H-pyrazol-3-ylamino)pyrazin-2-ylamino)phenyl)isobutyramide;
1-(2-methyl-5-(6-(5-methyl-1H-pyrazol-3-ylamino)pyrazin-2-ylamino)phenyl)pyrrolidin-2-one;
1-(2-methyl-5-(6-(5-methyl-1H-pyrazol-3-ylamino)pyrazin-2-ylamino)phenyl)piperidin-2-one;
N²-(3-(methoxymethyl)-4-methylphenyl)-N⁶-(5-methyl-1H-pyrazol-3-yl)pyrazine-2,6-diamine;
(2-methyl-5-(6-(5-methyl-1H-pyrazol-3-ylamino)pyrazin-2-ylamino)phenyl)methanol;
1-(7-(6-(5-methyl-1H-pyrazol-3-ylamino)pyrazin-2-ylamino)-3,4-dihydroquinolin-1(2H)-yl)ethanone;
Cyclopropyl(7-(6-(5-methyl-1H-pyrazol-3-ylamino)pyrazin-2-ylamino)-3,4-dihydroquinolin-1(2H)-yl)methanone;
Cyclopentyl(7-(6-(5-methyl-1H-pyrazol-3-ylamino)pyrazin-2-ylamino)-3,4-dihydroquinolin-1(2H)-yl)methanone;
Cyclopropyl(7-(6-(5-methyl-1H-pyrazol-3-ylamino)pyrazin-2-ylamino)-3,4-dihydroisoquinolin-2(1H)-yl)methanone;
Cyclopentyl(7-(6-(5-methyl-1H-pyrazol-3-ylamino)pyrazin-2-ylamino)-3,4-dihydroisoquinolin-2(1H)-yl)methanone;
2-(7-(6-(5-methyl-1H-pyrazol-3-ylamino)pyrazin-2-ylamino)-3,4-dihydroisoquinolin-2(1H)-yl)ethanol;
2-methoxy-N-(2-methyl-5-(6-(5-methyl-1H-pyrazol-3-ylamino)pyrazin-2-ylamino)phenyl)acetamide;
2-hydroxy-N-(2-methyl-5-(6-(5-methyl-1H-pyrazol-3-ylamino)pyrazin-2-ylamino)phenyl)acetamide;
3-hydroxy-N-(2-methyl-5-(6-(5-methyl-1H-pyrazol-3-ylamino)pyrazin-2-ylamino)phenyl)propanamide;
N²-(4-methoxyphenyl)-N⁶-(5-methyl-1H-pyrazol-3-yl)pyrazine-2,6-diamine;
N²-(5-methyl-1H-pyrazol-3-yl)-N⁶-p-tolylpyrazine-2,6-diamine;
6-(indolin-1-yl)-N-(5-methyl-1H-pyrazol-3-yl)pyrazin-2-amine;
5-(6-(5-methyl-1H-pyrazol-3-ylamino)pyrazin-2-ylamino)-2,3-dihydro-1H-inden-1-one;
6-(6-(5-methyl-1H-pyrazol-3-ylamino)pyrazin-2-ylamino)indolin-2-one;
N2-(isoindolin-5-yl)-N⁶-(5-methyl-1H-pyrazol-3-yl)pyrazine-2,6-diamine;
N²-(1H-indazol-6-yl)-N⁶-(5-methyl-1H-pyrazol-3-yl)pyrazine-2,6-diamine;
N²-(1H-benzo[d]imidazol-6-yl)-N⁶-(5-methyl-1H-pyrazol-3-yl)pyrazine-2,6-diamine;
Methyl 7-(6-(5-methyl-1H-pyrazol-3-ylamino)pyrazin-2-ylamino)-3,4-dihydroquinoline-1(2H)-carboxylate;
Methyl 7-(6-(5-methyl-1H-pyrazol-3-ylamino)pyrazin-2-ylamino)-3,4-dihydroisoquinoline-2(1H)-carboxylate;
2-(7-(6-(5-methyl-1H-pyrazol-3-ylamino)pyrazin-2-ylamino)-3,4-dihydroquinolin-1(2H)-yl)ethanol;
N²-(1-ethyl-1,2,3,4-tetrahydroquinolin-7-yl)-N⁶-(5-methyl-1H-pyrazol-3-yl)pyrazine-2,6-diamine;
7-(6-(5-methyl-1H-pyrazol-3-ylamino)pyrazin-2-ylamino)-3,4-dihydroquinolin-2(1H)-one;
N-methyl-7-(6-(5-methyl-1H-pyrazol-3-ylamino)pyrazin-2-ylamino)-3,4-dihydroquinoline-1(2H)-carboxamide;
N-methyl-7-(6-(5-methyl-1H-pyrazol-3-ylamino)pyrazin-2-ylamino)-3,4-dihydroisoquinoline-2(1H)-carboxamide;
N²-(2-ethyl-1,2,3,4-tetrahydroisoquinolin-7-yl)-N⁶-(5-methyl-1H-pyrazol-3-yl)pyrazine-2,6-diamine;
2-(7-(6-(5-methyl-1H-pyrazol-3-ylamino)pyrazin-2-ylamino)-3,4-dihydroquinolin-1(2H)-yl)-2-oxoethyl acetate;
2-hydroxy-1-(7-(6-(5-methyl-1H-pyrazol-3-ylamino)pyrazin-2-ylamino)-3,4-dihydroquinolin-1(2H)-yl)ethanone;
2-hydroxy-1-(7-(6-(5-methyl-1H-pyrazol-3-ylamino)pyrazin-2-ylamino)-3,4-dihydroisoquinolin-2(1H)-yl)ethanone;
2-(dimethylamino)-1-(7-(6-(5-methyl-1H-pyrazol-3-ylamino)pyrazin-2-ylamino)-3,4-dihydroquinolin-1(2H)-yl)ethanone;
2-methoxy-1-(7-(6-(5-methyl-1H-pyrazol-3-ylamino)pyrazin-2-ylamino)-3,4-dihydroisoquinolin-2(1H)-yl)ethanone;
2(1H-pyrazol-3-ylamino)pyrazin-2-ylamino)-3,4-dihydroisoquinolin-2(1H)-yl)-2-(methylamino)ethanone;
2-hydroxy-2-methyl-1-(7-(6-(5-methyl-1H-pyrazol-3-ylamino)pyrazin-2-ylamino)-3,4-dihydroisoquinolin-2(1H)-yl)propan-1-one;
2-amino-1-(7-(6-(5-methyl-1H-pyrazol-3-ylamino)pyrazin-2-ylamino)-3,4-dihydroisoquinolin-2(1H)-yl)ethanone;
Methyl 6-(6-(5-methyl-1H-pyrazol-3-ylamino)pyrazin-2-ylamino)indoline-1-carboxylate;
Methyl 5-(6-(5-methyl-1H-pyrazol-3-ylamino)pyrazin-2-ylamino)-2-(trifluoromethyl)phenylcarbamate;
N²-(4-chloro-3-(2-(dimethylamino)ethoxy)phenyl)-N⁶-(5-methyl-1H-pyrazol-3-yl)pyrazine-2,6-diamine;
1-(2-methyl-5-(6-(5-methyl-1H-pyrazol-3-ylamino)pyrazin-2-ylamino)phenyl)imidazolidin-2-one;
2-chloro-N,N-dimethyl-5-(6-(5-methyl-1H-pyrazol-3-ylamino)pyrazin-2-ylamino)benzamide;
2-chloro-N-methyl-5-(6-(5-methyl-1H-pyrazol-3-ylamino)pyrazin-2-ylamino)benzamide;
N-benzyl-2-chloro-5-(6-(5-methyl-1H-pyrazol-3-ylamino)pyrazin-2-ylamino)benzamide;
2-chloro-5-(6-(5-methyl-1H-pyrazol-3-ylamino)pyrazin-2-ylamino)-N-phenylbenzamide;
N-(3-(6-(5-methyl-1H-pyrazol-3-ylamino)pyrazin-2-ylamino)phenyl)methanesulfonamide;
N-(3-(6-(5-methyl-1H-pyrazol-3-ylamino)pyrazin-2-ylamino)phenyl)benzenesulfonamide;
Methyl 3-(6-(5-methyl-1H-pyrazol-3-ylamino)pyrazin-2-ylamino)phenylcarbamate;
Benzyl 3-(6-(5-methyl-1H-pyrazol-3-ylamino)pyrazin-2-ylamino)phenylcarbamate;
1-(3-(6-(5-methyl-1H-pyrazol-3-ylamino)pyrazin-2-ylamino)phenyl)-3-propylurea;
1-(3-(6-(5-methyl-1H-pyrazol-3-ylamino)pyrazin-2-ylamino)phenyl)-3-phenylurea;
N-(5-methyl-1H-pyrazol-3-yl)-6-phenylpyrazin-2-amine;
3-(6-(5-methyl-1H-pyrazol-3-ylamino)pyrazin-2-yl)benzonitrile;
6-(4-chlorophenyl)-N-(5-methyl-1H-pyrazol-3-yl)pyrazin-2-amine;
6-(3-chlorophenyl)-N-(5-methyl-1H-pyrazol-3-yl)pyrazin-2-amine;
6-(4-methoxyphenyl)-N-(5-methyl-1H-pyrazol-3-yl)pyrazin-2-amine;
6-(3-methoxyphenyl)-N-(5-methyl-1H-pyrazol-3-yl)pyrazin-2-amine;
N-(5-methyl-1H-pyrazol-3-yl)-6-p-tolylpyrazin-2-amine;
N-(5-methyl-1H-pyrazol-3-yl)-6-m-tolylpyrazin-2-amine;
6-(4-(dimethylamino)phenyl)-N-(5-methyl-1H-pyrazol-3-yl)pyrazin-2-amine;
3-(4-chlorophenylamino)-N-((1S,2S)-2-hydroxycyclopentyl)-5-(5-methyl-1H-pyrazol-3-ylamino)pyrazine-2-carboxamide;
3-(4-chlorophenylamino)-N-((1R,2R)-2-hydroxycyclopentyl)-5-(5-methyl-1H-pyrazol-3-ylamino)pyrazine-2-carboxamide;
(3-(4-chlorophenylamino)-5-(5-methyl-1H-pyrazol-3-ylamino)pyrazin-2-yl)(4-(hydroxymethyl)piperidin-1-yl)methanone;
(R)-3-(4-chlorophenylamino)-5-(5-methyl-1H-pyrazol-3-ylamino)-N-((5-oxopyrrolidin-2-yl)methyl)pyrazine-2-carboxamide;
(S)-3-(4-chlorophenylamino)-5-(5-methyl-1H-pyrazol-3-ylamino)-N-((5-oxopyrrolidin-2-yl)methyl)pyrazine-2-carboxamide;
(R)-3-(4-chlorophenylamino)-5-(5-methyl-1H-pyrazol-3-ylamino)-N-(pyrrolidin-2-ylmethyl)pyrazine-2-carboxamide;
(S)-3-(4-chlorophenylamino)-5-(5-methyl-1H-pyrazol-3-ylamino)-N-(pyrrolidin-2-ylmethyl)pyrazine-2-carboxamide;
3-(4-chlorophenylamino)-5-(5-methyl-1H-pyrazol-3-ylamino)pyrazine-2-carboxamide;
3-(4-chlorophenylamino)-5-(5-methyl-1H-pyrazol-3-ylamino)-N-(pyrrolidin-3-ylmethyl)pyrazine-2-carboxamide;
N-(2-chloro-5-(6-(5-methyl-1H-pyrazol-3-ylamino)-3-propionylpyrazin-2-ylamino)phenyl)acetamide;
(R)-1-(3-(4-chlorophenylamino)-5-(5-methyl-1H-pyrazol-3-ylamino)pyrazine-2-carbonyl)pyrrolidine-2-carboxamide;
(S)-1-(3-(4-chlorophenylamino)-5-(5-methyl-1H-pyrazol-3-ylamino)pyrazine-2-carbonyl)pyrrolidine-2-carboxamide;
3-(4-chlorophenylamino)-N-((1s,4s)-4-hydroxycyclohexyl)-5-(5-methyl-1H-pyrazol-3-ylamino)pyrazine-2-carboxamide;
3-(4-chlorophenylamino)-N-((1r,4r)-4-hydroxycyclohexyl)-5-(5-methyl-1H-pyrazol-3-ylamino)pyrazine-2-carboxamide;
(R)-(3-(4-chlorophenylamino)-5-(5-methyl-1H-pyrazol-3-ylamino)pyrazin-2-yl)(3-hydroxypyrrolidin-1-yl)methanone;
(S)-(3-(4-chlorophenylamino)-5-(5-methyl-1H-pyrazol-3-ylamino)pyrazin-2-yl)(3-hydroxypyrrolidin-1-yl)methanone;
(R)-3-(4-chlorophenylamino)-5-(5-methyl-1H-pyrazol-3-ylamino)-N-(pyrrolidin-3-yl)pyrazine-2-carboxamide;
(S)-3-(4-chlorophenylamino)-5-(5-methyl-1H-pyrazol-3-ylamino)-N-(pyrrolidin-3-yl)pyrazine-2-carboxamide;
3-(4-chlorophenylamino)-5-(5-methyl-1H-pyrazol-3-ylamino)-N-(piperidin-4-ylmethyl)pyrazine-2-carboxamide;
3-(4-chlorophenylamino)-5-(5-methyl-1H-pyrazol-3-ylamino)-N-(piperidin-2-ylmethyl)pyrazine-2-carboxamide;
(S)-3-(4-chlorophenylamino)-5-(5-methyl-1H-pyrazol-3-ylamino)-N-(piperidin-3-ylmethyl)pyrazine-2-carboxamide;
(R)-3-(4-chlorophenylamino)-5-(5-methyl-1H-pyrazol-3-ylamino)-N-(piperidin-3-ylmethyl)pyrazine-2-carboxamide;
(R)-(3-aminopyrrolidin-1-yl)(3-(4-chlorophenylamino)-5-(5-methyl-1H-pyrazol-3-ylamino)pyrazin-2-yl)methanone;
(S)-(3-aminopyrrolidin-1-yl)(3-(4-chlorophenylamino)-5-(5-methyl-1H-pyrazol-3-ylamino)pyrazin-2-yl)methanone;
Methyl 5-(3-(dimethylcarbamoyl)-6-(5-methyl-1H-pyrazol-3-ylamino)pyrazin-2-ylamino)-2-methylphenylcarbamate;
Methyl 5-(3-((1R,2R)-2-hydroxycyclopentylcarbamoyl)-6-(5-methyl-1H-pyrazol-3-ylamino)pyrazin-2-ylamino)-2-methylphenylcarbamate;
Methyl 5-(3-((1S,2S)-2-hydroxycyclopentylcarbamoyl)-6-(5-methyl-1H-pyrazol-3-ylamino)pyrazin-2-ylamino)-2-methylphenylcarbamate;
(S)-methyl 5-(3-(2-(aminomethyl)pyrrolidine-1-carbonyl)-6-(5-methyl-1H-pyrazol-3-ylamino)pyrazin-2-ylamino)-2-methylphenylcarbamate;
(R)-methyl 5-(3-(2-(aminomethyl)pyrrolidine-1-carbonyl)-6-(5-methyl-1H-pyrazol-3-ylamino)pyrazin-2-ylamino)-2-methylphenylcarbamate;
(R)-methyl 2-methyl-5-(6-(5-methyl-1H-pyrazol-3-ylamino)-3-(pyrrolidin-2-ylmethylcarbamoyl)pyrazin-2-ylamino)phenylcarbamate;
(S)-methyl 2-methyl-5-(6-(5-methyl-1H-pyrazol-3-ylamino)-3-(pyrrolidin-2-ylmethylcarbamoyl)pyrazin-2-ylamino)phenylcarbamate;
Methyl 5-(3-(2-(dimethylamino)ethylcarbamoyl)-6-(5-methyl-1H-pyrazol-3-ylamino)pyrazin-2-ylamino)-2-methylphenylcarbamate;
3-(4-chlorophenylamino)-N-(2-hydroxyethyl)-5-(5-methyl-1H-pyrazol-3-ylamino)pyrazine-2-carboxamide;
(3-(4-chlorophenylamino)-5-(5-methyl-1H-pyrazol-3-ylamino)pyrazin-2-yl)(piperazin-1-yl)methanone;
3-(4-chlorophenylamino)-N,N-dimethyl-5-(5-methyl-1H-pyrazol-3-ylamino)pyrazine-2-carboxamide;
1-(4-(3-(4-chlorophenylamino)-5-(5-methyl-1H-pyrazol-3-ylamino)pyrazine-2-carbonyl)piperazin-1-yl)ethanone;
(3-(4-chlorophenylamino)-5-(5-methyl-1H-pyrazol-3-ylamino)pyrazin-2-yl)(4-(2-hydroxyethyl)piperazin-1-yl)methanone;
3-(4-chlorophenylamino)-N-methyl-5-(5-methyl-1H-pyrazol-3-ylamino)pyrazine-2-carboxamide;
(S)-(3-(4-chlorophenylamino)-5-(5-methyl-1H-pyrazol-3-ylamino)pyrazin-2-yl)(2-(hydroxymethyl)pyrrolidin-1-yl)methanone;
(R)-(3-(4-chlorophenylamino)-5-(5-methyl-1H-pyrazol-3-ylamino)pyrazin-2-yl)(2-(hydroxymethyl)pyrrolidin-1-yl)methanone;
N-(2-acetamidoethyl)-3-(4-chlorophenylamino)-5-(5-methyl-1H-pyrazol-3-ylamino)pyrazine-2-carboxamide;
N-(2-amino-2-oxoethyl)-3-(4-chlorophenylamino)-5-(5-methyl-1H-pyrazol-3-ylamino)pyrazine-2-carboxamide;
N-(2-aminoethyl)-3-(4-chlorophenylamino)-5-(5-methyl-1H-pyrazol-3-ylamino)pyrazine-2-carboxamide;
4-(3-(4-chlorophenylamino)-5-(5-methyl-1H-pyrazol-3-ylamino)pyrazine-2-carbonyl)piperazin-2-one;
(3-(4-chlorophenylamino)-5-(5-methyl-1H-pyrazol-3-ylamino)pyrazin-2-yl)(3-(hydroxymethyl)pyrrolidin-1-yl)methanone;
N-(1-(3-(4-chlorophenylamino)-5-(5-methyl-1H-pyrazol-3-ylamino)pyrazine-2-carbonyl)piperidin-4-yl)acetamide;
3-(4-chlorophenylamino)-5-(5-methyl-1H-pyrazol-3-ylamino)-N-(2-(methylamino)ethyl)pyrazine-2-carboxamide;
3-(4-chlorophenylamino)-5-(5-methyl-1H-pyrazol-3-ylamino)-N-(piperidin-4-yl)pyrazine-2-carboxamide;
(3-(4-chlorophenylamino)-5-(5-methyl-1H-pyrazol-3-ylamino)pyrazin-2-yl)(3-(hydroxymethyl)piperidin-1-yl)methanone;
(4-aminopiperidin-1-yl)(3-(4-chlorophenylamino)-5-(5-methyl-1H-pyrazol-3-ylamino)pyrazin-2-yl)methanone;
N-(2-amino-2-oxoethyl)-3-(4-chlorophenylamino)-N-methyl-5-(5-methyl-1H-pyrazol-3-ylamino)pyrazine-2-carboxamide;
(R)-(3-(4-chlorophenylamino)-5-(5-methyl-1H-pyrazol-3-ylamino)pyrazin-2-yl)(3-hydroxypiperidin-1-yl)methanone;
(S)-(3-(4-chlorophenylamino)-5-(5-methyl-1H-pyrazol-3-ylamino)pyrazin-2-yl)(3-hydroxypiperidin-1-yl)methanone;
(R)-(3-aminopiperidin-1-yl)(3-(4-chlorophenylamino)-5-(5-methyl-1H-pyrazol-3-ylamino)pyrazin-2-yl)methanone;
(S)-(3-aminopiperidin-1-yl)(3-(4-chlorophenylamino)-5-(5-methyl-1H-pyrazol-3-ylamino)pyrazin-2-yl)methanone;
3-(4-chlorophenylamino)-N-methyl-5-(5-methyl-1H-pyrazol-3-ylamino)-N-(2-(methylamino)ethyl)pyrazine-2-carboxamide;
(4-(2-aminoethyl)piperazin-1-yl)(3-(4-chlorophenylamino)-5-(5-methyl-1H-pyrazol-3-ylamino)pyrazin-2-yl)methanone;
N-(azetidin-3-yl)-3-(4-chlorophenylamino)-5-(5-methyl-1H-pyrazol-3-ylamino)pyrazine-2-carboxamide;
(S)-(2-(aminomethyl)pyrrolidin-1-yl)(3-(4-chlorophenylamino)-5-(5-methyl-1H-pyrazol-3-ylamino)pyrazin-2-yl)methanone;
(R)-(2-(aminomethyl)pyrrolidin-1-yl)(3-(4-chlorophenylamino)-5-(5-methyl-1H-pyrazol-3-ylamino)pyrazin-2-yl)methanone;
(R)-3-(4-chlorophenylamino)-5-(5-methyl-1H-pyrazol-3-ylamino)-N-(piperidin-3-yl)pyrazine-2-carboxamide;
(S)-3-(4-chlorophenylamino)-5-(5-methyl-1H-pyrazol-3-ylamino)-N-(piperidin-3-yl)pyrazine-2-carboxamide;
Methyl 5-(3-(isopropylcarbamoyl)-6-(5-methyl-1H-pyrazol-3-ylamino)pyrazin-2-ylamino)-2-methylphenylcarbamate; or
Methyl 2-methyl-5-(6-(5-methyl-1H-pyrazol-3-ylamino)-3-(pyrrolidine-1-carbonyl)pyrazin-2-ylamino)phenylcarbamate.

35. The compound of claim 1, wherein the compound at a concentration of 10 μM inhibits IKK2 by at least about 50%.

36. A pharmaceutical composition comprising an effective amount of a compound of claim 1.

37. The pharmaceutical composition of claim 36 suitable for oral, parenteral, mucosal, transdermal or topical administration.

38. A method for treating or preventing inflammatory conditions, immunological conditions, cancer, neurodegenerative diseases, age-related diseases, cardiovascular diseases and metabolic conditions, or conditions treatable or preventable by inhibition of an IKK, or an IKK pathway, comprising administering to a patient an effective amount of a compound of claim 1.

39. The method of claim 38, wherein the condition treatable or preventable by inhibition of an IKK, or an IKK pathway is selected from rheumatoid arthritis; rheumatoid spondylitis; osteoarthritis; gout; asthma; bronchitis; allergic rhinitis; chronic obstructive pulmonary disease; cystic fibrosis; inflammatory bowel disease; irritable bowel syndrome; mucous colitis; ulcerative colitis; Crohn's disease; Huntington's disease; gastritis; esophagitis; hepatitis; pancreatitis; nephritis; multiple sclerosis; lupus erythematosus; Type II diabetes; obesity; atherosclerosis; restenosis following angioplasty; left ventricular hypertrophy; myocardial infarction; stroke; ischemic damages of heart, lung, gut, kidney, liver, pancreas, spleen or brain; acute or chronic organ transplant rejection; preservation of the organ for transplantation; organ failure or loss of limb; graft versus host disease; endotoxin shock; multiple organ failure; sepsis; Guillain-Barre syndrome; psoriasis; burn from exposure to fire, chemicals or radiation; eczema; dermatitis; skin graft; ischemia; ischemic conditions associated with surgery or traumatic injury; epilepsy; Alzheimer's disease; Parkinson's disease; immunological response to bacterial or viral infection; cachexia; muscle atrophy; angiogenic diseases, proliferative diseases; solid tumors; or cancers of the colon, rectum, prostate, liver, lung, bronchus, pancreas, brain, head, neck, stomach, skin, kidney, cervix, blood, larynx, esophagus, mouth, pharynx, urinary bladder, ovary or uterine.

40. The method of claim 38, wherein the inflammatory condition is psoriasis, asthma, allergic rhinitis, bronchitis, chronic obstructive pulmonary disease, sepsis, reperfusion injury, cystic fibrosis, inflammatory bowel disease, irritable bowel syndrome, Crohn's disease, mucous colitis, ulcerative colitis, toxic shock syndrome, acute or chronic pain, thermal injury, adult respiratory distress syndrome (ARDS), multiple organ injury secondary to trauma, acute glomerulonephritis, dermatoses with acute inflammatory components, acute purulent meningitis, myasthenia gravis, scleroderma, atopic dermatitis, steatohepatitis, diabetes, or obesity.

41. The method of claim 38, wherein the immunological condition is rheumatoid arthritis, rheumatoid spondylitis, osteoarthritis, multiple sclerosis, lupus, inflammatory bowel disease, ulcerative colitis, Guillain-Barre Syndrome, Crohn's disease, psoriasis, graft versus host disease, myasthenia gravis, Grave's disease or diabetes.

42. The method of claim 38, wherein the cancer is leukemia, lymphoma, myeloma, myelodysplastic syndrome, or cancers of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system, solid tumors or blood borne tumors.

43. A method of inhibiting an IKK-2 in a cell expressing IKK-2, comprising contacting said cell with an effective amount of a compound of claim 1.
